(19) United States
(12) Patent Application Publication
(10) Pub. No.: US 2025/0386665 A1
(43) Pub. Date: Dec. 18, 2025

(54) COMPOSITION FOR ORGANIC ELECTRONIC DEVICES

(71) Applicant: Merck Patent GmbH, Darmstadt (DE)

(72) Inventors: Amir Hossain PARHAM, Darmstadt (DE); Philipp STOESSEL, Darmstadt (DE); Christian EHRENREICH, Darmstadt (DE)

(21) Appl. No.: 18/877,347

(22) PCT Filed: Jun. 22, 2023
(86) PCT No.: PCT/EP2023/066894

§ 371 Date: Dec. 20, 2024

(30) Foreign Application Priority Data
Jun. 24, 2022 (EP) 22181087.2

(51) Int. Cl. H10K 50/16; C07D 251/22; C09K 11/06; H10K 50/11; H10K 50/15; H10K 71/16; H10K 85/30; H10K 85/60

(52) U.S. Cl. CPC H10K 50/16; C07D 251/22; C09K 11/06; H10K 50/11; H10K 50/15; H10K 71/16; H10K 85/342; H10K 85/346; H10K 85/622; H10K 85/633; H10K 85/636; H10K 85/654; H10K 85/6572; H10K 85/6574; H10K 85/6576; C09K 2211/1029

(57) ABSTRACT

The present invention relates to a composition comprising an electron-transporting host and a hole-transporting host, to the use thereof in electronic devices and to electronic devices containing said composition. The electron-transporting host corresponds to a compound of formula (1) from the class of N-bridged triphenylenes that contain a substituted pyridine, pyrimidine or triazine moiety bonded via the N atom.

The present invention relates to a composition comprising an electron-transporting host and a hole-transporting host, to the use thereof in electronic devices and electronic devices comprising said composition. The electron-transporting host corresponds to a compound of the formula (1) from the class of the N-bridged triphenylenes containing a substituted pyridine, pyrimidine or triazine unit bonded via the nitrogen atom.

The structure of organic electroluminescent devices (e.g. OLEDs—organic light-emitting diodes or OLECs—organic light-emitting electrochemical cells) in which organic semiconductors are used as functional materials has long been known. Emitting materials used here, aside from fluorescent emitters, are increasingly organometallic complexes which exhibit phosphorescence rather than fluorescence. For quantum-mechanical reasons, up to a fourfold increase in energy efficiency and power efficiency is possible using organometallic compounds as phosphorescent emitters. In general terms, however, there is still a need for improvement in OLEDs, especially also in OLEDs which exhibit triplet emission (phosphorescence), for example with regard to efficiency, operating voltage and lifetime.

The properties of organic electroluminescent devices are not only determined by the emitters used. Also of particular significance here are especially the other materials used, such as host and matrix materials, hole blocker materials, electron transport materials, hole transport materials and electron or exciton blocker materials, and among these especially the host or matrix materials. Improvements to these materials can lead to distinct improvements to electroluminescent devices.

Host materials for use in organic electronic devices are well known to the person skilled in the art. The term “matrix material” is also frequently used in the prior art when what is meant is a host material for phosphorescent emitters. This use of the term is also applicable to the present invention. In the meantime, a multitude of host materials has been developed both for fluorescent and for phosphorescent electronic devices.

U.S. Pat. No. 6,392,250 B1 discloses the use of a mixture consisting of an electron transport material, a hole transport material and a fluorescent emitter in the emission layer of an OLED. With the aid of this mixture, it was possible to improve the lifetime of the OLED compared to the prior art.

U.S. Pat. No. 6,803,720 B1 discloses the use of a mixture comprising a phosphorescent emitter and a hole transport material and an electron transport material in the emission layer of an OLED. Both the hole transport material and the electron transport material are small organic molecules.

A further means of improving the performance data of electronic devices, especially of organic electroluminescent devices, is to use combinations of two or more materials, especially host materials or matrix materials.

WO 2012/048781 gives the first description of N-bridged triphenylenes having electron- and hole-transporting properties that are used in a green-phosphorescing OLED in the emission layer as hole-transporting host and/or electron-transporting host and/or in the hole transport layer as hole transport material.

CN112961145 describes biscarbazole derivatives with O-bridged triphenylenes as substituent on the nitrogen atom of one of the carbazoles. These find use in green-phosphorescing OLEDs.

However, there is still need for improvement in the case of use of these materials or in the case of use of mixtures of the materials, especially in relation to efficiency, operating voltage and/or lifetime of the organic electroluminescent device.

CN1156269 A with filing date Nov. 4, 2022, published Jan. 20, 2023, discloses similar compounds.

A problem addressed by the present invention is therefore that of providing a combination of materials which are suitable for use in an organic electroluminescent device, especially in a fluorescent or phosphorescent OLED, and lead to good device properties, especially with regard to an improved lifetime, and that of providing the corresponding electroluminescent device.

It has now been found that this problem is solved, and the drawbacks from the prior art are eliminated, by the combination of at least one compound of the formula (1) and at least one hole-transporting compound of the formula (2) or of the formula (3) or of the formula (4) or of the formula (5) in an organic layer of an organic electroluminescent device. The use of such a material combination for production of an organic layer in an organic electroluminescent device leads to very good properties of these devices, especially with regard to lifetime, especially with equal or improved efficiency and/or operating voltage. The advantages are especially also manifested in the presence of a light-emitting component in the emission layer, especially in the case of combination with emitters of the formula (IIIa) or emitters of the formulae (I) to (VI) at concentrations between 2% and 20% by weight, especially concentrations of 6% by weight and 12% by weight.

The present invention therefore firstly provides a composition containing at least one compound of the formula (1) and at least one compound of the formula (2) or of the formula (3) or of the formula (4) or of the formula (5):

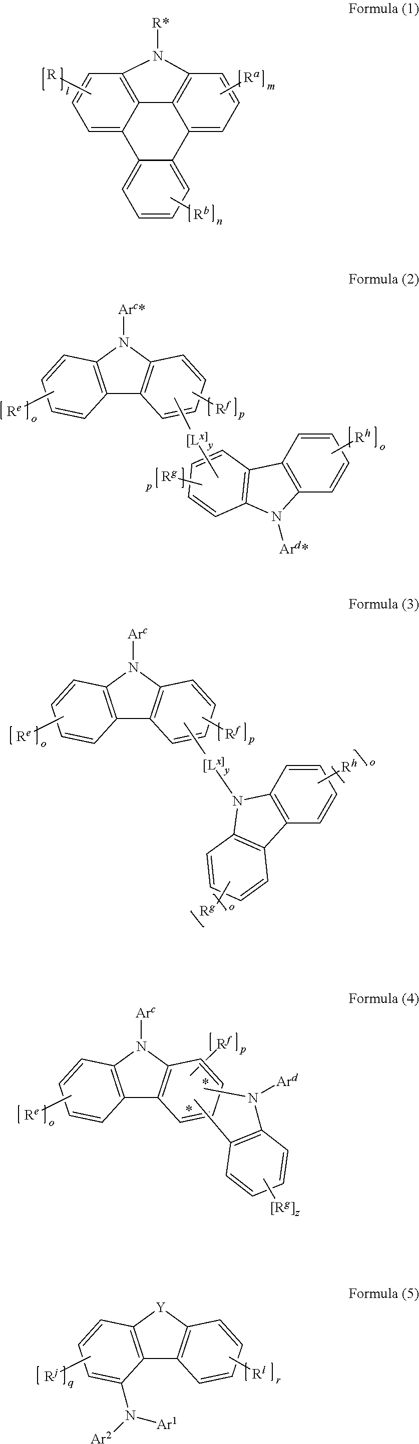

where the symbols and indices used are as follows:
R* is a group of the following formula (1a):

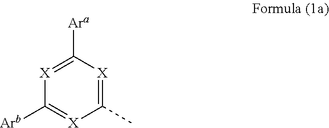

where the dashed bond represents the bond to the nitrogen atom in formula (1);
X is the same or different at each instance and is N or CR^{c}, with the proviso that at least one X group is N and, if X is CR^{c}, this does not form a ring with Ar^{a} or Ar^{b};
Ar^{a}, Ar^{b} are the same or different at each instance and are each independently an aromatic ring system having 6 to 40 aromatic ring atoms or a heteroaromatic ring system having 5 to 40 aromatic ring atoms, each of which may be substituted by one or more R¹ radicals;
L^{x} is the same or different at each instance and is an aromatic ring system having 6 to 40 aromatic ring atoms or a heteroaromatic ring system having 5 to 40 aromatic ring atoms, which may be substituted in each case by one or more R¹ radicals;
Ar^{c}, Ar^{d} are the same or different at each instance and are each independently an aromatic ring system having 6 to 40 aromatic ring atoms or a heteroaromatic ring system having 5 to 40 aromatic ring atoms, each of which may be substituted by one or more R^{d} radicals;
Ar^{c*}, Ar^{d*} are the same or different at each instance and are each independently an aromatic ring system which has 6 to 40 aromatic ring atoms and may be substituted in each case by one or more R^{d} radicals or a heteroaryl group selected from the group consisting of dibenzofuran, dibenzothiophene, carbazole, triphenyleno[1,2-bcd]thiophene, substituted naphtho[1,2,3,4,def]carbazole, phenoxazine, phenothiazine, indolo[3,2,1-jk]carbazole, biscarbazole, benzcarbazole, indenocarbazole, indolocarbazole, benzofurocarbazole, benzothioenocarbazole, dihydroacridine, dihydrophenazine, dibenzodioxin, thianthrene, phenoxathiine, each of which may be substituted by one or more R^{d} radicals, where, independently of one another, an R^{f} radical or an R^{e} radical together with an Ar^{c*} radical or an R^{g} radical or an R^{h} radical together with an Ar^{d*} radical may form a monocyclic or polycyclic, aliphatic, aromatic or heteroaromatic ring, or a group of the formula (6a) or (6b), where the dashed bond represents the bond to the nitrogen atom in the formula (2);

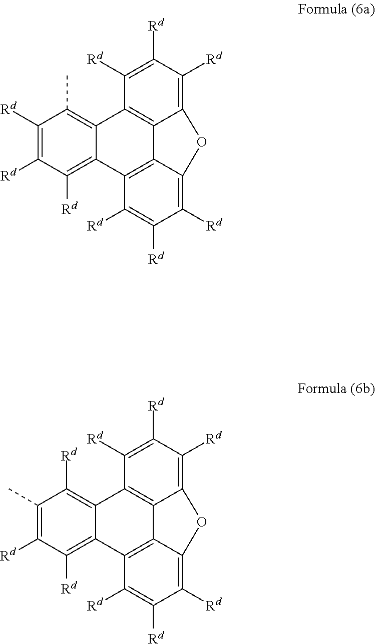

Y is the same or different at each instance and is selected from O, S and C(R^{k})₂;
Ar¹, Ar² are the same or different at each instance and are each independently an aromatic ring system having 6 to 40 aromatic ring atoms or a heteroaromatic ring system having 5 to 40 aromatic ring atoms, each of which may be substituted by one or more R¹ radicals;
R, R^{a}, R^{b} are the same or different at each instance and are each independently H, D, F, Cl, Br, I, N(Ar′)₂, N(R¹)₂, OAr′, SAr′, B(OR¹)₂, CHO, C(═O)R¹, CR¹═C(R¹)₂, CN, C(═O)OR¹, C(═O)NR¹, Si(R¹)₃, NO₂, P(═O)(R¹)₂, OSO₂R¹, OR¹, S(═O)R¹, S(═O)₂R¹, SR¹, a straight-chain alkyl group having 1 to 20 carbon atoms or an alkenyl or alkynyl group having 2 to 20 carbon atoms or a branched or cyclic alkyl group having 3 to 20 carbon atoms, where the alkyl, alkenyl or alkynyl group may in each case be substituted by one or more R¹ radicals, where one or more nonadjacent CH₂ groups may be replaced by —R¹C═CR¹—, —C≡C—, Si(R¹)₂, CONR¹, C═O, C═S, —C(═O)O—, P(═O)(R¹), O, S, SO or SO₂, or an aromatic, heteroaromatic or aromatic ring system having 5 to 40 aromatic ring atoms, each of which may be substituted by one or more R¹ radicals, where two or more R and/or R^{a} and/or R^{b} radicals bonded to the same cycle may together form an aliphatic, heteroaliphatic or aromatic ring system that may be substituted by one or more R¹ radicals, and where two R and/or R^{a} and/or R^{b} radicals bonded to the same carbon, silicon, germanium or tin atom may together form a monocyclic or polycyclic, aliphatic or aromatic ring system that may be substituted by one or more R¹ radicals;
R^{c} is the same or different at each instance and is H, D, F, Cl, Br, I, N(Ar′)₂, N(R¹)₂, OAr′, SAr′, B(OR¹)₂, CHO, C(═O)R¹, CR¹═C(R¹)₂, CN, C(═O)OR¹, C(═O)NR¹, Si(R¹)₃, NO₂, P(═O)(R¹)₂, OSO₂R¹, OR¹, S(═O)R¹, S(═O)₂R¹, SR¹, a straight-chain alkyl group having 1 to 20 carbon atoms or an alkenyl or alkynyl group having 2 to 20 carbon atoms or a branched or cyclic alkyl group having 3 to 20 carbon atoms, where the alkyl, alkenyl or alkynyl group may each case be substituted by one or more R¹ radicals, where one or more nonadjacent CH₂ groups may be replaced by —R¹C═CR¹—, —C≡C—, Si(R¹)₂, NR¹, CONR¹, C═O, C═S, —C(═O)O—, P(═O)(R¹), O, S, SO or SO₂, or an aromatic or heteroaromatic ring system having 5 to 40 aromatic ring atoms, each of which may be substituted by one or more R¹ radicals, where two or more R^{c}, R^{e}, R^{f}, R^{g}, R^{h} or Rⁱ radicals bonded to the same cycle may together form an aliphatic, heteroaliphatic, aromatic or heteroaromatic ring systems that may be substituted by one or more R¹ radicals, and where two R^{c}, R^{e}, R^{f}, R^{g}, R^{h} or Rⁱ radicals bonded to the same carbon, silicon, germanium or tin atom may together form a monocyclic or polycyclic, aliphatic, aromatic or heteroaromatic ring system that may be substituted by one or more R¹ radicals;
R^{d} is the same or different at each instance and is H, D, F, Cl, Br, I, N(Ar′)₂, N(R³)₂, OAr′, SAr′, B(OR³)₂, CHO, C(═O)R³, CR³═C(R³)₂, CN, C(═O)OR³, C(═O)NR³, Si(R³)₃, NO₂, P(═O)(R³)₂, OSO₂R³, S(═O)R³, S(═O)₂R³, SR³, a straight-chain alkyl group having 1 to 20 carbon atoms or an alkenyl or alkynyl group having 2 to 20 carbon atoms or a branched or cyclic alkyl group having 3 to 20 carbon atoms, where the alkyl, alkenyl or alkynyl group may be substituted in each case by one or more R¹ radicals, where one or more nonadjacent CH₂ groups may be replaced by —R³C═CR³—, —C≡C—, Si(R³)₂, NR³, CONR³, C═O, C═S, —C(═O)O—, P(═O)(R³), O, S, SO or SO₂, or an aromatic or heteroaromatic ring system which has 5 to 40 aromatic ring atoms and may be substituted in each case by one or more R³ radicals, where two or more R^{d} radicals bonded to the same cycle may together form an aliphatic, heteroaliphatic, aromatic or heteroaromatic ring system which may be substituted by one or more R¹ radicals, and where two R^{d} radicals bonded to the same carbon, silicon, germanium or tin atom may together form a monocyclic or polycyclic, aliphatic, aromatic or heteroaromatic ring system which may be substituted by one or more R³ radicals;
Ar′ is the same or different at each instance and is an aromatic ring system having 6 to 40 aromatic ring atoms or a heteroaromatic ring system having 5 to 40 aromatic ring atoms, which may be substituted by one or more R¹ radicals;
R^{e}, R^{f}, R^{g}, R^{h} are the same or different at each instance and are each independently H, D, F, Cl, Br, I, N(Ar′)₂, N(R¹)₂, OAr′, SAr′, B(OR¹)₂, CHO, C(═O)R¹, CR¹═C(R¹)₂, CN, C(═O)OR¹, C(═O)NR¹, Si(R¹)₃, NO₂, P(═O)(R¹)₂, OSO₂R¹, OR¹, S(═O)R¹, S(═O)₂R¹, SR¹, a straight-chain alkyl group having 1 to 20 carbon atoms or an alkenyl or alkynyl group having 2 to 20 carbon atoms or a branched or cyclic alkyl group having 3 to 20 carbon atoms, where the alkyl, alkenyl or alkynyl group may be substituted in each case by one or more R¹ radicals, where one or more nonadjacent CH₂ groups may be replaced by —R¹C═CR¹—, —C≡C—, Si(R¹)₂, NR¹, CONR¹, C═O, C═S, —C(═O)O—, P(═O)(R¹), O, S, SO or SO₂, or an aromatic or heteroaromatic ring system which has 5 to 40 aromatic ring atoms and may be substituted in each case by one or more R¹ radicals;
Rⁱ, R^{j} are the same or different at each instance and are each independently H, D, F, Cl, Br, I, N(Ar′)₂, N(R¹)₂, OAr′, SAr′, B(OR¹)₂, CHO, C(═O)R¹, CR¹═C(R¹)₂, CN, C(═O)OR¹, C(═O)NR¹, Si(R¹)₃, NO₂, P(═O)(R¹)₂, OSO₂R¹, OR¹, S(═O)R¹, S(═O)₂R¹, SR¹, a straight-chain alkyl group having 1 to 20 carbon atoms or an alkenyl or alkynyl group having 2 to 20 carbon atoms or a branched or cyclic alkyl group having 3 to 20 carbon atoms, where the alkyl, alkenyl or alkynyl group may in each case be substituted by one or more R¹ radicals, where one or more nonadjacent CH₂ groups may be replaced by —R¹C═CR¹—, —C≡C—, Si(R¹)₂, NR¹, CONR¹, C═O, C═S, —C(═O)O—, P(═O)(R¹), O, S, SO or SO₂, or an aromatic or heteroaromatic ring system having 5 to 40 aromatic ring atoms, each of which may be substituted by one or more R¹ radicals, where two or more Rⁱ or R^{j} radicals bonded to the same cycle may together form an aliphatic, heteroaliphatic, aromatic or heteroaromatic ring system that may be substituted by one or more R¹ radicals, and where two Rⁱ or R^{j} radicals bonded to the same carbon, silicon, germanium or tin atom may together form a monocyclic or polycyclic, aliphatic, aromatic or heteroaromatic ring system that may be substituted by one or more R¹ radicals;
R^{k}, R¹ are the same or different at each instance and are D, F, I, B(OR²)₂, N(R²)₂, CHO, C(═O)R², CR²═C(R²)₂, CN, C(═O)OR², Si(R²)₃, NO₂, P(═O)(R²)₂, OSO₂R², SR², OR², S(═O)R², S(═O)₂R², a straight-chain alkyl group having 1 to 20 carbon atoms or an alkenyl or alkynyl group having 2 to 20 carbon atoms or a branched or cyclic alkyl group having 3 to 20 carbon atoms, where the alkyl, alkenyl or alkynyl group may in each case be substituted by one or more R² radicals and where one or more CH₂ groups in the abovementioned groups may be replaced by —R²C═CR²—, —C≡C—, Si(R²)₂, C═O, C═S, —C(═O)O—, NR², CONR², P(═O)(R²), O, S, SO or SO₂, and where one or more hydrogen atoms in the abovementioned groups may be replaced by D, F, Cl, Br, I, CN or NO₂, or an aromatic or heteroaromatic ring system which has 5 to 30 aromatic ring atoms and may be substituted in each case by one or more R² radicals, where two or more R^{k} or R¹ radicals together may form an aliphatic, heteroaliphatic, aromatic or heteroaromatic ring system;
R² is the same or different at each instance and is D, F, CN or an aliphatic, aromatic or heteroaromatic organic radical having 1 to 20 carbon atoms, in which one or more hydrogen atoms may also be replaced by D or F; at the same time, two or more R² substituents may be joined to one another and may form a ring;
R³ is the same or different at each instance and is D, CN or an aliphatic, aromatic or heteroaromatic organic radical having 1 to 20 carbon atoms; at the same time, two or more R³ substituents may be joined to one another and may form a ring;
l, m, p, q are the same or different at each instance and are independently 0, 1, 2 or 3;
n, o, r, z are the same or different at each instance and are independently 0, 1, 2, 3 or 4;
y at each instance is independently 0 or 1.

An aryl group in the context of this invention contains 6 to 40 carbon atoms; a heteroaryl group in the context of this invention contains 2 to 39 carbon atoms and at least one heteroatom, with the proviso that the sum total of carbon atoms and heteroatoms is at least 5. The heteroatoms are preferably selected from N, O and/or S. An aryl group or heteroaryl group is understood here to mean either a simple aromatic cycle, i.e. benzene, or a simple heteroaromatic cycle, for example pyridine, pyrimidine, thiophene, etc., or a fused (annelated) aryl or heteroaryl group, for example naphthalene, anthracene, phenanthrene, quinoline, isoquinoline, etc. Aromatics joined to one another by a single bond, for example biphenyl, by contrast, are not referred to as an aryl or heteroaryl group but as an aromatic ring system. An aromatic ring system in the context of this invention contains 6 to 40 carbon atoms in the ring system. A heteroaromatic ring system in the context of this invention contains 2 to 39 carbon atoms and at least one heteroatom in the ring system, with the proviso that the sum total of carbon atoms and heteroatoms is at least 5. The heteroatoms are preferably selected from N, O and/or S. An aromatic or heteroaromatic ring system in the context of this invention shall be understood to mean a system which does not necessarily contain only aryl or heteroaryl groups, but in which it is also possible for two or more aryl or heteroaryl groups to be joined by a non-aromatic unit, for example a carbon, nitrogen or oxygen atom. These shall likewise be understood to mean systems in which two or more aryl or heteroaryl groups are joined directly to one another, for example biphenyl, terphenyl, bipyridine or phenylpyridine. For example, systems such as fluorene, 9,9′-spirobifluorene, 9,9-diarylfluorene, triarylamine, diaryl ethers, stilbene, etc. shall also be regarded as aromatic ring systems in the context of this invention, and likewise systems in which two or more aryl groups are joined, for example, by a short alkyl group. Preferred aromatic or heteroaromatic ring systems are simple aryl or heteroaryl groups and groups in which two or more aryl or heteroaryl groups are joined directly to one another, for example biphenyl or bipyridine, and also fluorene or spirobifluorene.

An electron-rich heteroaromatic ring system is characterized in that it is a heteroaromatic ring system containing no electron-deficient heteroaryl groups. An electron-deficient heteroaryl group is a six-membered heteroaryl group having at least one having at least one nitrogen atom or a five-membered heteroaryl group having at least two heteroatoms, one of which is a nitrogen atom and the other is oxygen, sulfur or a substituted nitrogen atom, where further aryl or heteroaryl groups may also be fused onto these groups in each case. By contrast, electron-rich heteroaryl groups our five-membered heteroaryl groups having exactly one heteroatom selected from oxygen, sulfur and substituted nitrogen, to which may be fused further aryl groups and/or further electron-rich five-membered heteroaryl groups. Thus, examples of electron-rich heteroaryl groups are pyrrole, furan, thiophene, indole, benzofuran, benzothiophene, carbazole, dibenzofuran, dibenzothiophene or indenocarbazole. An electron-rich heteroaryl group is also referred to as an electron-rich heteroaromatic radical.

An electron-deficient heteroaromatic ring system is characterized in that it contains at least one electron-deficient heteroaryl group, and especially preferably no electron-rich heteroaryl groups.

In the context of the present invention, the term “alkyl group” is used as an umbrella term both for linear and branched alkyl groups and for cyclic alkyl groups. Analogously, the terms “alkenyl group” and “alkynyl group” are used as umbrella terms both for linear or branched alkenyl or alkynyl groups and for cyclic alkenyl or alkynyl groups.

In the context of the present invention, an aliphatic hydrocarbyl radical or an alkyl group or an alkenyl or alkynyl group which may contain 1 to 20 carbon atoms and in which individual hydrogen atoms or CH₂ groups may also be substituted by the abovementioned groups is preferably understood to mean the methyl, ethyl, n-propyl, i-propyl, n-butyl, i-butyl, s-butyl, t-butyl, 2-methylbutyl, n-pentyl, s-pentyl, neopentyl, cyclopentyl, n-hexyl, neohexyl, cyclohexyl, n-heptyl, cycloheptyl, n-octyl, cyclooctyl, 2-ethylhexyl, trifluoromethyl, pentafluoroethyl, 2,2,2-trifluoroethyl, ethenyl, propenyl, butenyl, pentenyl, cyclopentenyl, hexenyl, cyclohexenyl, heptenyl, cycloheptenyl, octenyl, cyclooctenyl, ethynyl, propynyl, butynyl, pentynyl, hexynyl, heptynyl or octynyl radicals. An alkoxy group OR¹ having 1 to 40 carbon atoms is preferably understood to mean methoxy, trifluoromethoxy, ethoxy, n-propoxy, i-propoxy, n-butoxy, i-butoxy, s-butoxy, t-butoxy, n-pentoxy, s-pentoxy, 2-methylbutoxy, n-hexoxy, cyclohexyloxy, n-heptoxy, cycloheptyloxy, n-octyloxy, cyclooctyloxy, 2-ethylhexyloxy, pentafluoroethoxy and 2,2,2-trifluoroethoxy. A thioalkyl group SR¹ having 1 to 40 carbon atoms is understood to mean especially methylthio, ethylthio, n-propylthio, i-propylthio, n-butylthio, i-butylthio, s-butylthio, t-butylthio, n-pentylthio, s-pentylthio, n-hexylthio, cyclohexylthio, n-heptylthio, cycloheptylthio, n-octylthio, cyclooctylthio, 2-ethylhexylthio, trifluoromethylthio, pentafluoroethylthio, 2,2,2-trifluoroethylthio, ethenylthio, propenylthio, butenylthio, pentenylthio, cyclopentenylthio, hexenylthio, cyclohexenylthio, heptenylthio, cycloheptenylthio, octenylthio, cyclooctenylthio, ethynylthio, propynylthio, butynylthio, pentynylthio, hexynylthio, heptynylthio or octynylthio. In general, alkyl, alkoxy or thioalkyl groups according to the present invention may be straight-chain, branched or cyclic, where one or more nonadjacent CH₂ groups may be replaced by the abovementioned groups; in addition, it is also possible for one or more hydrogen atoms to be replaced by D, F, Cl, Br, I, CN or NO₂, preferably F, Cl or CN, more preferably F or CN.

An aromatic ring system which has 6 to 40 aromatic ring atoms or a heteroaromatic ring system which has 5-40 aromatic ring atoms and may also be substituted in each case by the abovementioned R¹ radicals or a hydrocarbyl radical and which may be joined to the aromatic or heteroaromatic system via any desired positions is understood to mean especially groups derived from benzene, naphthalene, anthracene, benzanthracene, phenanthrene, pyrene, chrysene, perylene, fluoranthene, naphthacene, pentacene, benzopyrene, biphenyl, biphenylene, terphenyl, triphenylene, fluorene, spirobifluorene, dihydrophenanthrene, dihydropyrene, tetrahydropyrene, cis- or trans-indenofluorene, cis- or trans-indenocarbazole, cis- or trans-indolocarbazole, truxene, isotruxene, spirotruxene, spiroisotruxene, furan, benzofuran, isobenzofuran, dibenzofuran, thiophene, benzothiophene, isobenzothiophene, dibenzothiophene, pyrrole, indole, isoindole, carbazole, pyridine, quinoline, isoquinoline, acridine, phenanthridine, benzo-5,6-quinoline, benzo-6,7-quinoline, benzo-7,8-quinoline, phenothiazine, phenoxazine, pyrazole, indazole, imidazole, benzimidazole, naphthimidazole, phenanthrimidazole, pyridimidazole, pyrazinimidazole, quinoxalinimidazole, oxazole, benzoxazole, naphthoxazole, anthroxazole, phenanthroxazole, isoxazole, 1,2-thiazole, 1,3-thiazole, benzothiazole, pyridazine, hexaazatriphenylene, benzopyridazine, pyrimidine, benzopyrimidine, quinoxaline, 1,5-diazaanthracene, 2,7-diazapyrene, 2,3-diazapyrene, 1,6-diazapyrene, 1,8-diazapyrene, 4,5-diazapyrene, 4,5,9,10-tetraazaperylene, pyrazine, phenazine, phenoxazine, phenothiazine, fluorubine, naphthyridine, azacarbazole, benzocarboline, phenanthroline, 1,2,3-triazole, 1,2,4-triazole, benzotriazole, 1,2,3-oxadiazole, 1,2,4-oxadiazole, 1,2,5-oxadiazole, 1,3,4-oxadiazole, 1,2,3-thiadiazole, 1,2,4-thiadiazole, 1,2,5-thiadiazole, 1,3,4-thiadiazole, 1,3,5-triazine, 1,2,4-triazine, 1,2,3-triazine, tetrazole, 1,2,4,5-tetrazine, 1,2,3,4-tetrazine, 1,2,3,5-tetrazine, purine, pteridine, indolizine and benzothiadiazole, or groups derived from a combination of these systems.

The wording that two or more radicals together may form a ring system, in the context of the present description, should be understood to mean, inter alia, that the two radicals are joined to one another by a chemical bond with formal elimination of two hydrogen atoms. This is illustrated by the following scheme:

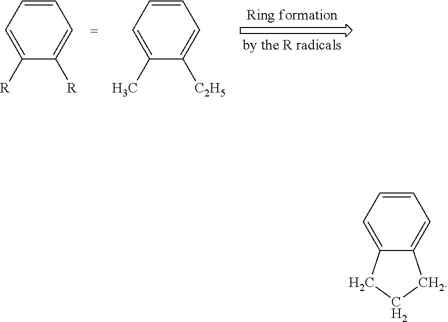

In addition, however, the abovementioned wording shall also be understood to mean that, if one of the two radicals is hydrogen, the second radical binds to the position to which the hydrogen atom was bonded, forming a ring. This will be illustrated by the following scheme:

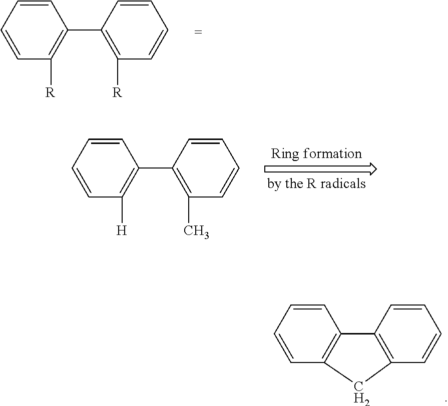

In respect of the indices l, m and n and the radicals R, R^{a} and R^{b}, the R radical shall occur l times, the R^{a} radical m times and the R^{b} radical n times, and all other positions on the base skeleton of the compounds of the formula (1) shall be substituted by H or D, where l and m are the same or different at each instance and are each 0, 1, 2 or 3 and n is 0, 1, 2, 3 or 4.

The same applies to the indices and radicals in the formulae (2), (3), (4) and (5).

The invention further provides a process for producing the organic electroluminescent devices and mixtures comprising at least one compound of the formula (1) and at least one compound of the formula (2) or of the formula (3) or of the formula (4) or of the formula (5), and specific material combinations.

The corresponding preferred embodiments as described hereinafter likewise form part of the subject-matter of the present invention. The surprising and advantageous effects are achieved through specific selection of the compounds of the formula (1) and the compounds of the formula (2) or formula (3) or of the formula (4) or of the formula (5).

The organic electronic device of the invention is, for example, an organic integrated circuit (OIC), an organic field-effect transistor (OFET), an organic thin-film transistor (OTFT), an organic solar cell (OSC), an organic optical detector, an organic photoreceptor, an organic light-emitting transistors (OLET), an organic field-quench device (OFQD), an organic light-emitting electrochemical cell (OLEC, LEC, LEEC), an organic laser diode (O-laser), or an organic light-emitting diode (OLED). The electronic device is preferably an electroluminescent device or, synonymously, a light-emitting device.

The organic electroluminescent device of the invention is, for example, an organic light-emitting transistor (OLET), an organic field quench device (OFQD), an organic light-emitting electrochemical cell (OLEC, LEC, LEEC), an organic laser diode (O-laser) or an organic light-emitting diode (OLED). The organic electroluminescent device of the invention is especially an organic light-emitting diode or an organic light-emitting electrochemical cell. The device of the invention is more preferably an OLED.

The organic layer of the device of the invention containing the material combination of at least one compound of the formula (1) and at least one compound of the formula (2) or of the formula (3) or of the formula (4) or of the formula (5) as described above or described hereinafter preferably comprises, as organic layer, a light-emitting layer (EML), a hole injection layer (HIL), a hole transport layer (HTL), an electron transport layer (ETL), an electron injection layer (EIL) and/or a hole blocker layer (HBL). It is also possible for the device of the invention to include multiple layers from this group selected from EML, HIL, HTL, ETL, EIL and HBL. Particular preference is given to the material combination of at least one compound of the formula (1) and at least one compound of the formula (2) or of the formula (3) or of the formula (4) or of the formula (5), as described above or described hereinafter, in the EML together with a fluorescent or phosphorescent emitter, especially with a phosphorescent emitter.

However, the device may also comprise inorganic materials or else layers formed entirely from inorganic materials.

It is preferable that the organic layer containing at least one compound of the formula (1) and at least one compound of the formula (2) or of the formula (3) or of the formula (4) or of the formula (5) is a light-emitting phosphorescent layer which is characterized in that it comprises, in addition to the material combination of the compounds of the formula (1) and formula (2) or of the formula (3) or of the formula (4) or of the formula (5) as described above, at least one phosphorescent emitter. A suitable selection of emitters and preferred emitters is described hereinafter.

A phosphorescent emitter in the context of the present invention is a compound that exhibits luminescence from an excited state with higher spin multiplicity, i.e. a spin state >1, especially from an excited triplet state. In the context of this application, all luminescent complexes with transition metals or lanthanides are to be regarded as phosphorescent emitters. A more exact definition is given hereinafter.

When the materials of the organic layer comprising at least one compound of the formula (1) as described above or described as preferred hereinafter and at least one compound of the formula (2) or of the formula (3) or of the formula (4) or of the formula (5) as described above or described hereinafter is used in the light-emitting layer as host or matrix material for a phosphorescent emitter, it is preferable when the triplet energy thereof is greater than or equal to, but not significantly less than, the triplet energy of the phosphorescent emitter. In respect of the triplet level, it is preferably the case that T₁(emitter)−T₁(matrix)≤0.2 eV, more preferably ≤0.15 eV, most preferably ≤0.1 eV. T₁(matrix) here is the triplet level of the host material in the emission layer, this condition being applicable to each of the two host materials, and T₁(emitter) is the triplet level of the phosphorescent emitter. If the emission layer contains more than two matrix materials, the abovementioned relationship is preferably also applicable to every further matrix material.

In a preferred embodiment of the invention, the composition consists of a compound of the formula (1) in combination with a compound of the formula (2) or of the formula (3) or of the formula (4) or of the formula (5).

There follows a description of the material of the formula (1) and its preferred embodiments that is/are present in the device of the invention. The preferred embodiments of the material 1 of the formula (1) are also applicable to the mixture and/or a formulation of the invention.

In a preferred embodiment of the formula (1a), at least two X are N and the third X is CR^{c}; in a preferred embodiment of the formula (1a), all three X are N. Preferred embodiments of the compounds of the formula (1) are accordingly compounds in which the formula (1a) represents a formula (1b), (1c) or (1d), more preferably the formula (1b) or (1c), especially the formula (1b). In a further preferred embodiment, R^{c} in the formulae (1c) or (1d) is H or D.

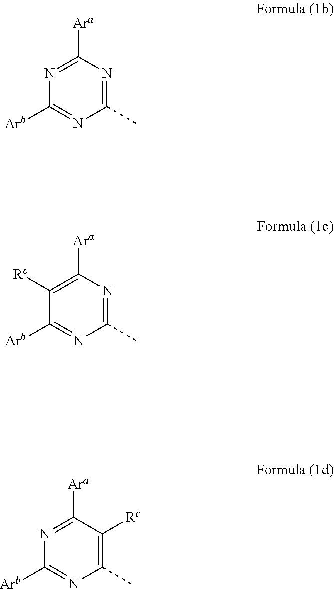

In a preferred embodiment of the formula (1), the index l, m and n is 0, 1, 2 or 3, more preferably 0 or 1; in particular, the sum total of the indices m+n+l is 0 or 1. If the R, R^{a} and R^{b} radicals are D, the sum total of the indices is preferably l+m+n=10. The R* group in the formulae (1-1a) to (1-1t) preferably represents the formulae (1b), (1c) or (1d), more preferably formula (1b). Preferred embodiments are the following compounds of the formulae (1-1a) to (1-1t):

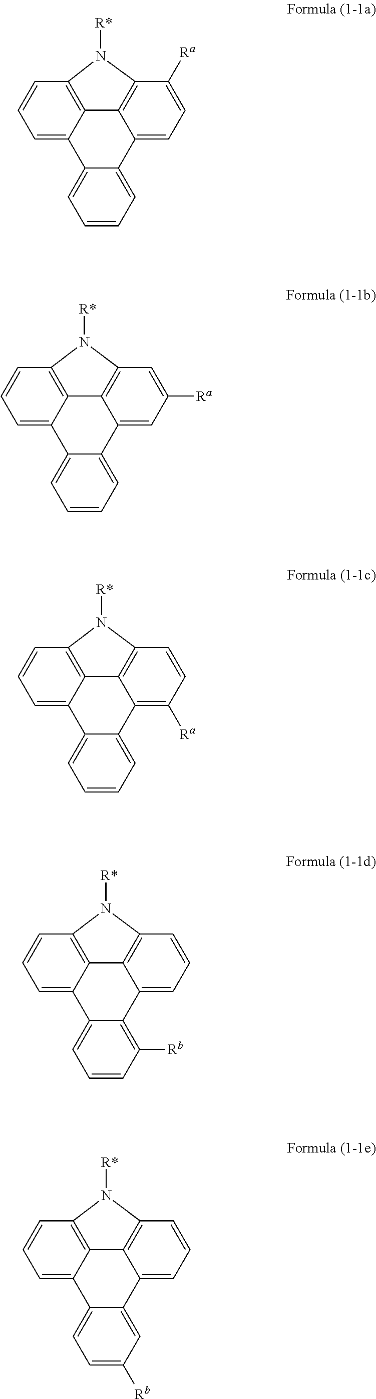

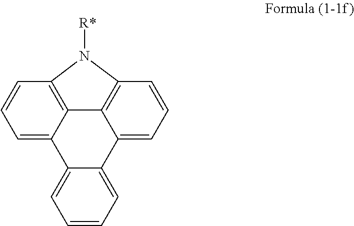

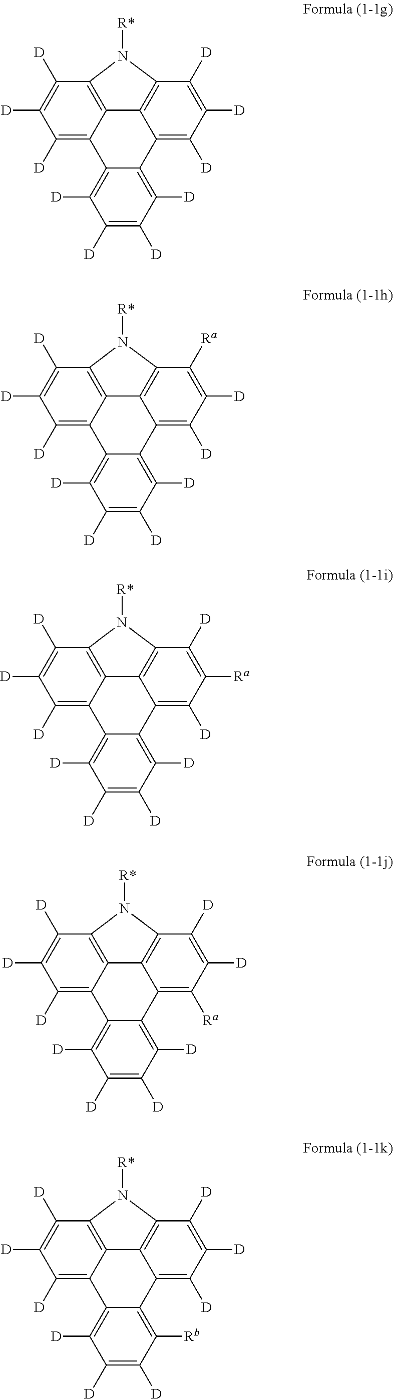

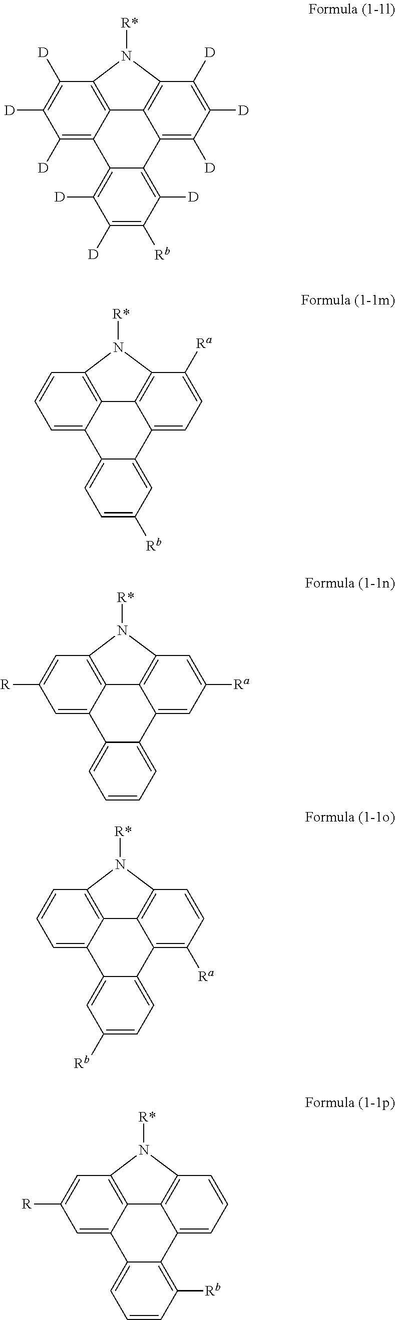

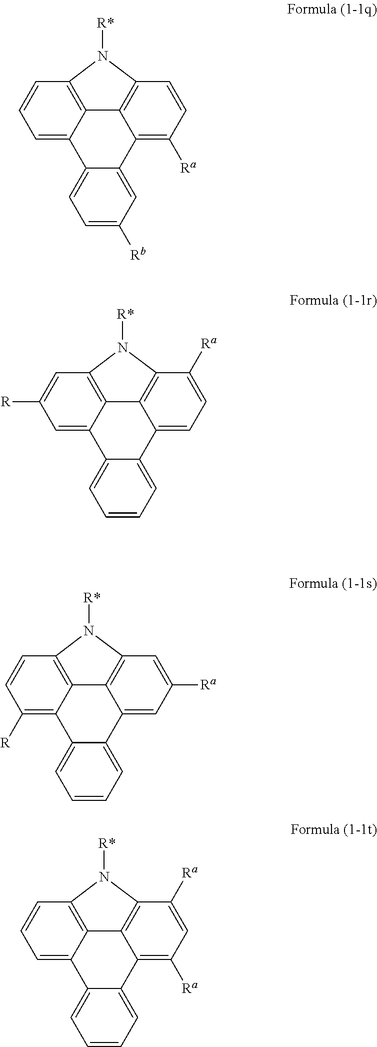

where the symbols used have the definitions given above.

In a preferred embodiment of the formula (1a), Ar^{a} and Ar^{b} are the same or different at each instance and are an aromatic ring system having 6 to 30 aromatic ring atoms or a heteroaromatic ring system having 5 to 30 aromatic ring atoms, more preferably an aromatic ring system having 6 to 24 aromatic ring atoms or a heteroaromatic ring system having 5 to 24 aromatic ring atoms, each of which may be substituted by one or more R¹ radicals, especially an aromatic ring system having 6 to 14 aromatic ring atoms or a heteroaromatic ring system having 5 to 14 aromatic ring atoms.

In a preferred embodiment of the formula (1a), the Ar^{a} and Ar^{b} radicals in the compounds of the formula (1) are different.

Examples of suitable compounds of the formula (1) that are selected in accordance with the invention are the structures shown below in Table 1.

TABLE 1

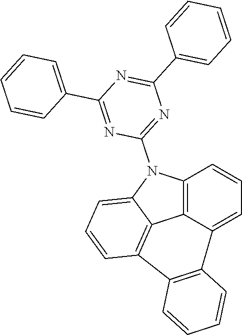 [SMILES: c1ccc(-c2nc(-c3ccccc3)nc(-n3c4cccc5c6ccccc6c6cccc3c6c54)n2)cc1]

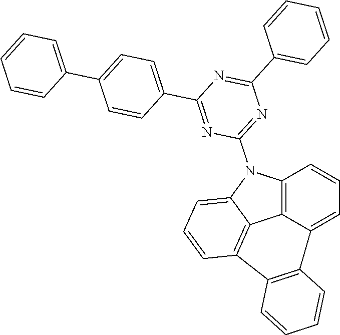 [SMILES: c1ccc(-c2ccc(-c3nc(-c4ccccc4)nc(-n4c5cccc6c7ccccc7c7cccc4c7c65)n3)cc2)cc1]

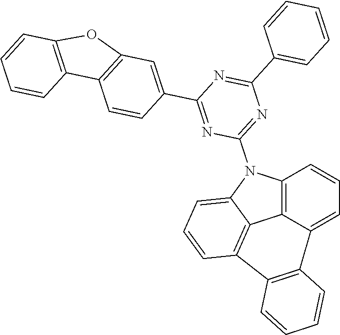 [SMILES: c1ccc(-c2nc(-c3ccc4c(c3)oc3ccccc34)nc(-n3c4cccc5c6ccccc6c6cccc3c6c54)n2)cc1]

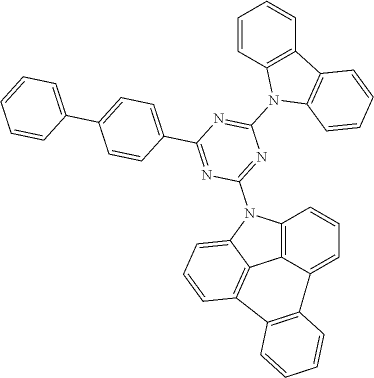 [SMILES: c1ccc(-c2ccc(-c3nc(-n4c5ccccc5c5ccccc54)nc(-n4c5cccc6c7ccccc7c7cccc4c7c65)n3)cc2)cc1]

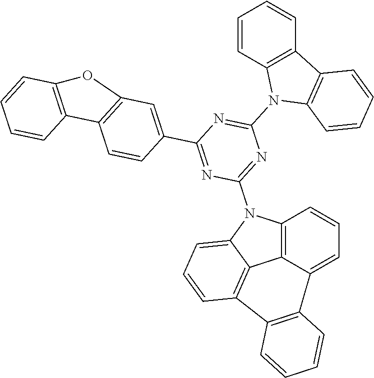 [SMILES: c1ccc2c(c1)oc1cc(-c3nc(-n4c5ccccc5c5ccccc54)nc(-n4c5cccc6c7ccccc7c7cccc4c7c65)n3)ccc12]

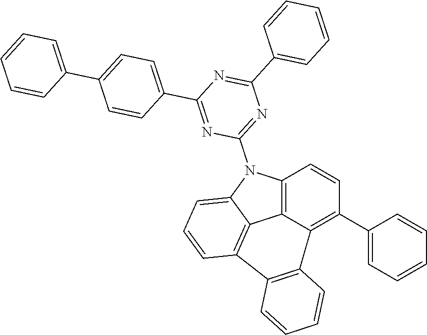 [SMILES: c1ccc(-c2ccc(-c3nc(-c4ccccc4)nc(-n4c5cccc6c7ccccc7c7c(-c8ccccc8)ccc4c7c65)n3)cc2)cc1]

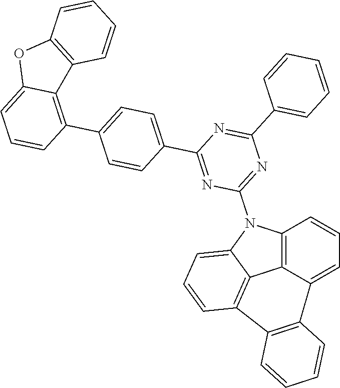 [SMILES: c1ccc(-c2nc(-c3ccc(-c4cccc5oc6ccccc6c45)cc3)nc(-n3c4cccc5c6ccccc6c6cccc3c6c54)n2)cc1]

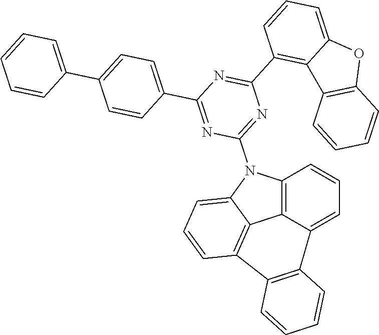 [SMILES: c1ccc(-c2ccc(-c3nc(-c4cccc5oc6ccccc6c45)nc(-n4c5cccc6c7ccccc7c7cccc4c7c65)n3)cc2)cc1]

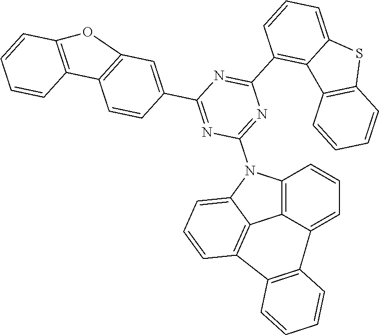 [SMILES: c1ccc2c(c1)oc1cc(-c3nc(-c4cccc5sc6ccccc6c45)nc(-n4c5cccc6c7ccccc7c7cccc4c7c65)n3)ccc12]

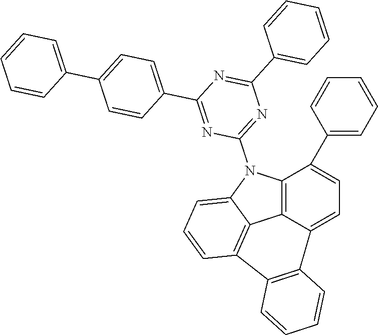 [SMILES: c1ccc(-c2ccc(-c3nc(-c4ccccc4)nc(-n4c5cccc6c7ccccc7c7ccc(-c8ccccc8)c4c7c65)n3)cc2)cc1]

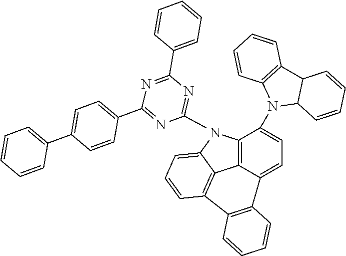 [SMILES: C1=CC2c3ccccc3N(c3ccc4c5ccccc5c5cccc6c5c4c3n6-c3nc(-c4ccccc4)nc(-c4ccc(-c5ccccc5)cc4)n3)C2C=C1]

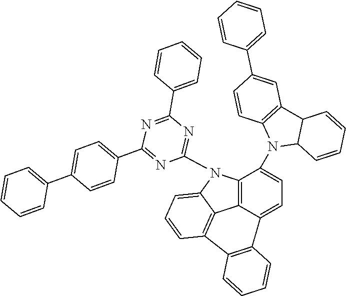 [SMILES: C1=CC2c3cc(-c4ccccc4)ccc3N(c3ccc4c5ccccc5c5cccc6c5c4c3n6-c3nc(-c4ccccc4)nc(-c4ccc(-c5ccccc5)cc4)n3)C2C=C1]

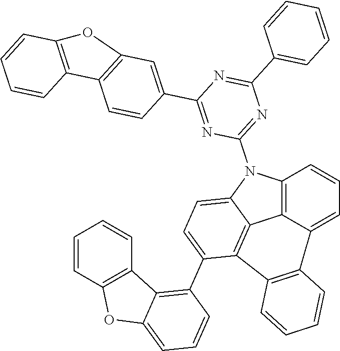 [SMILES: c1ccc(-c2nc(-c3ccc4c(c3)oc3ccccc34)nc(-n3c4cccc5c6ccccc6c6c(-c7cccc8oc9ccccc9c78)ccc3c6c54)n2)cc1]

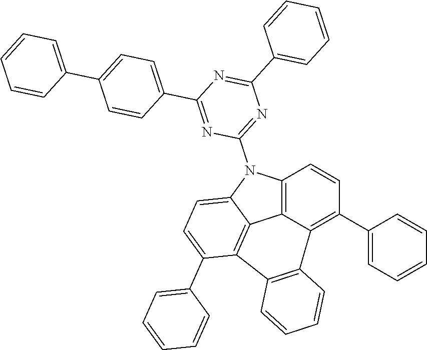 [SMILES: c1ccc(-c2ccc(-c3nc(-c4ccccc4)nc(-n4c5ccc(-c6ccccc6)c6c7ccccc7c7c(-c8ccccc8)ccc4c7c65)n3)cc2)cc1]

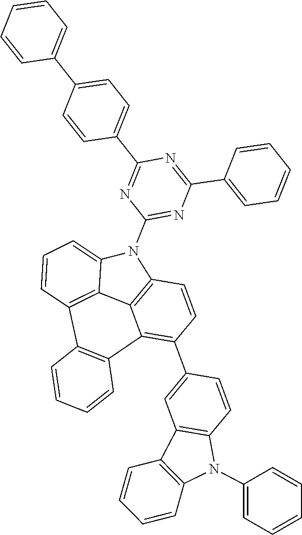 [SMILES: c1ccc(-c2ccc(-c3nc(-c4ccccc4)nc(-n4c5cccc6c7ccccc7c7c(-c8ccc9c(c8)c8ccccc8n9-c8ccccc8)ccc4c7c65)n3)cc2)cc1]

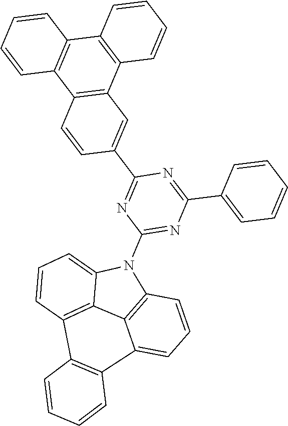 [SMILES: c1ccc(-c2nc(-c3ccc4c5ccccc5c5ccccc5c4c3)nc(-n3c4cccc5c6ccccc6c6cccc3c6c54)n2)cc1]

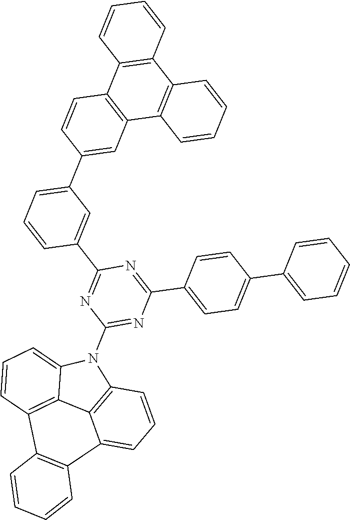 [SMILES: c1ccc(-c2ccc(-c3nc(-c4cccc(-c5ccc6c7ccccc7c7ccccc7c6c5)c4)nc(-n4c5cccc6c7ccccc7c7cccc4c7c65)n3)cc2)cc1]

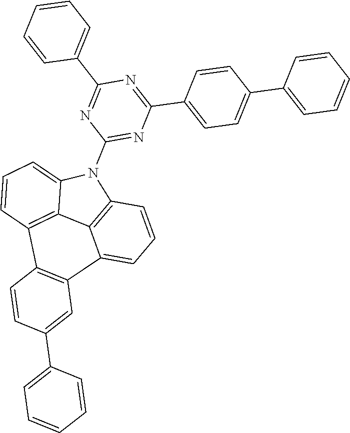 [SMILES: c1ccc(-c2ccc(-c3nc(-c4ccccc4)nc(-n4c5cccc6c7ccc(-c8ccccc8)cc7c7cccc4c7c65)n3)cc2)cc1]

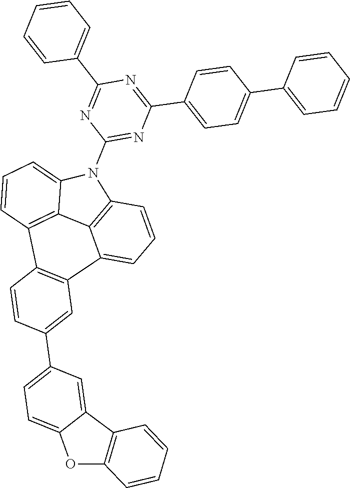 [SMILES: c1ccc(-c2ccc(-c3nc(-c4ccccc4)nc(-n4c5cccc6c7ccc(-c8ccc9oc%10ccccc%10c9c8)cc7c7cccc4c7c65)n3)cc2)cc1]

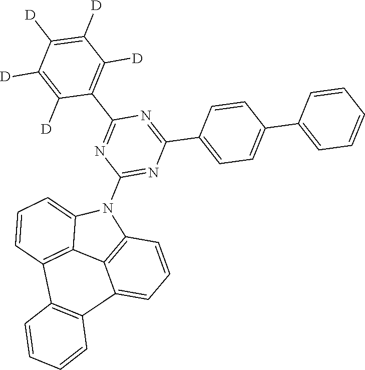 [SMILES: [2H]c1c([2H])c([2H])c(-c2nc(-c3ccc(-c4ccccc4)cc3)nc(-n3c4cccc5c6ccccc6c6cccc3c6c54)n2)c([2H])c1[2H]]

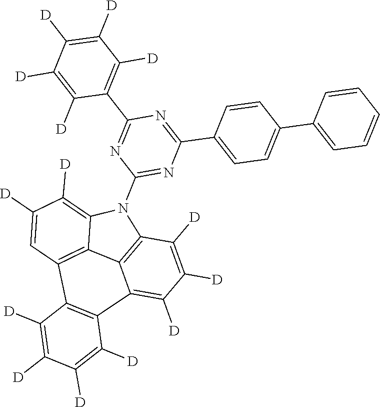 [SMILES: [2H]c1cc2c3c([2H])c([2H])c([2H])c([2H])c3c3c([2H])c([2H])c([2H])c4c3c2c(c1[2H])n4-c1nc(-c2ccc(-c3ccccc3)cc2)nc(-c2c([2H])c([2H])c([2H])c([2H])c2[2H])n1]

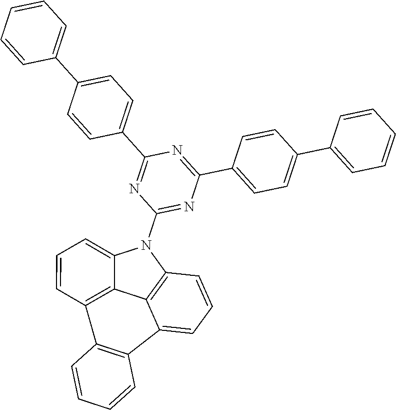 [SMILES: c1ccc(-c2ccc(-c3nc(-c4ccc(-c5ccccc5)cc4)nc(-n4c5cccc6c7ccccc7c7cccc4c7c65)n3)cc2)cc1]

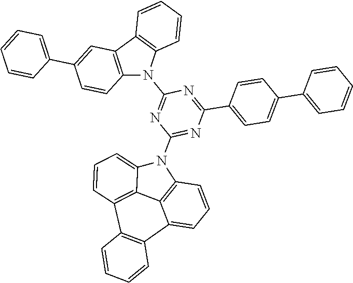 [SMILES: c1ccc(-c2ccc(-c3nc(-n4c5ccccc5c5cc(-c6ccccc6)ccc54)nc(-n4c5cccc6c7ccccc7c7cccc4c7c65)n3)cc2)cc1]

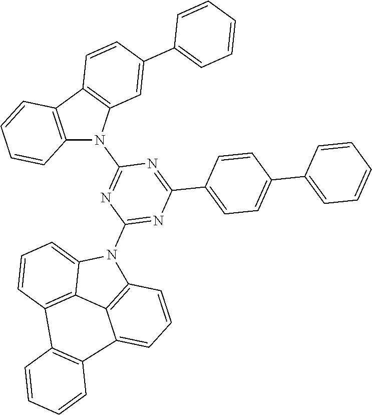 [SMILES: c1ccc(-c2ccc(-c3nc(-n4c5ccccc5c5ccc(-c6ccccc6)cc54)nc(-n4c5cccc6c7ccccc7c7cccc4c7c65)n3)cc2)cc1]

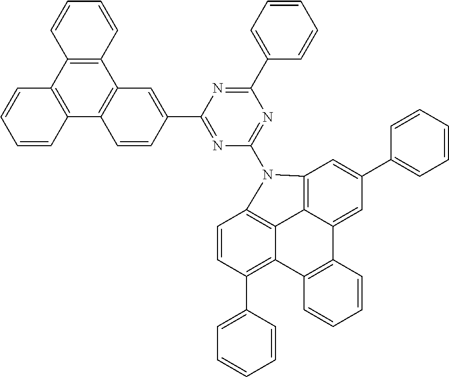 [SMILES: c1ccc(-c2cc3c4ccccc4c4c(-c5ccccc5)ccc5c4c3c(c2)n5-c2nc(-c3ccccc3)nc(-c3ccc4c5ccccc5c5ccccc5c4c3)n2)cc1]

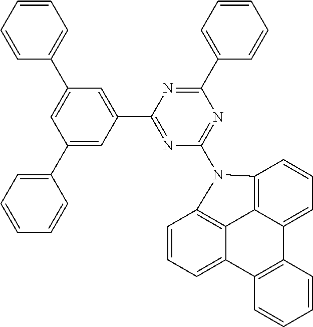 [SMILES: c1ccc(-c2cc(-c3ccccc3)cc(-c3nc(-c4ccccc4)nc(-n4c5cccc6c7ccccc7c7cccc4c7c65)n3)c2)cc1]

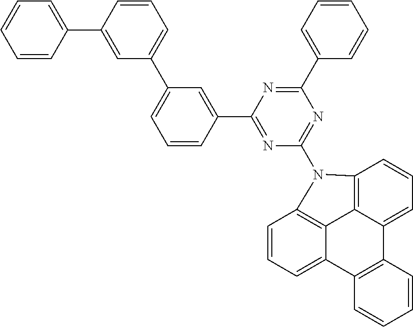 [SMILES: c1ccc(-c2cccc(-c3cccc(-c4nc(-c5ccccc5)nc(-n5c6cccc7c8ccccc8c8cccc5c8c76)n4)c3)c2)cc1]

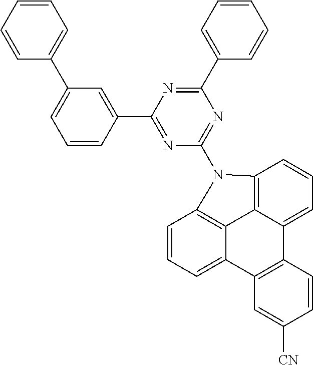 [SMILES: N#Cc1ccc2c(c1)c1cccc3c1c1c2cccc1n3-c1nc(-c2ccccc2)nc(-c2cccc(-c3ccccc3)c2)n1]

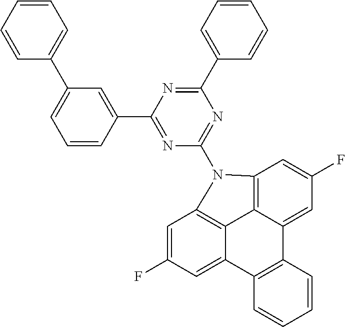 [SMILES: Fc1cc2c3ccccc3c3cc(F)cc4c3c2c(c1)n4-c1nc(-c2ccccc2)nc(-c2cccc(-c3ccccc3)c2)n1]

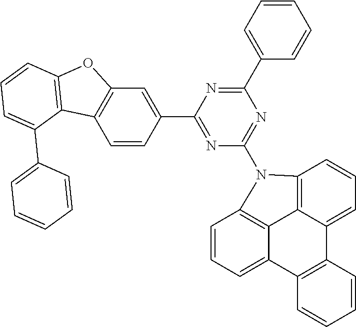 [SMILES: c1ccc(-c2nc(-c3ccc4c(c3)oc3cccc(-c5ccccc5)c34)nc(-n3c4cccc5c6ccccc6c6cccc3c6c54)n2)cc1]

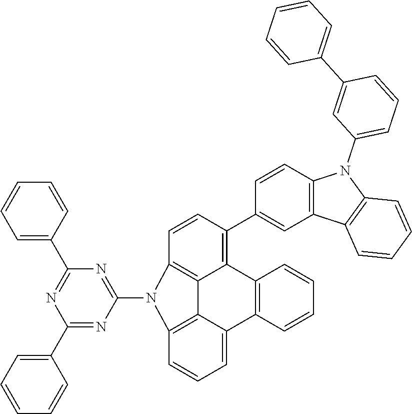 [SMILES: c1ccc(-c2cccc(-n3c4ccccc4c4cc(-c5ccc6c7c5c5ccccc5c5cccc(c57)n6-c5nc(-c6ccccc6)nc(-c6ccccc6)n5)ccc43)c2)cc1]

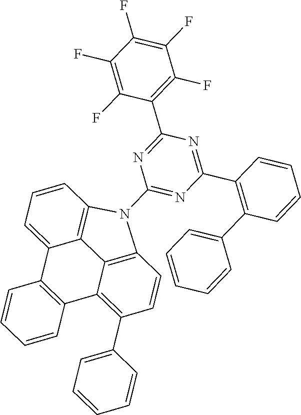 [SMILES: Fc1c(F)c(F)c(-c2nc(-c3ccccc3-c3ccccc3)nc(-n3c4cccc5c6ccccc6c6c(-c7ccccc7)ccc3c6c54)n2)c(F)c1F]

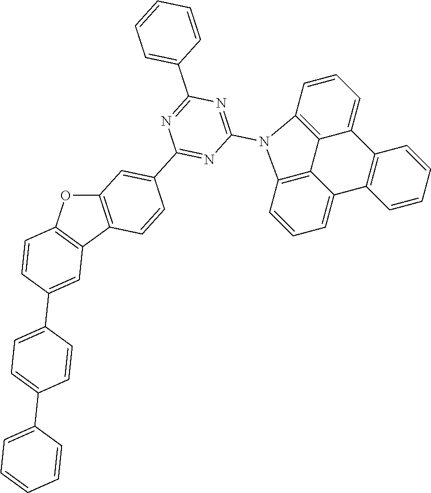 [SMILES: c1ccc(-c2ccc(-c3ccc4oc5cc(-c6nc(-c7ccccc7)nc(-n7c8cccc9c%10ccccc%10c%10cccc7c%10c98)n6)ccc5c4c3)cc2)cc1]

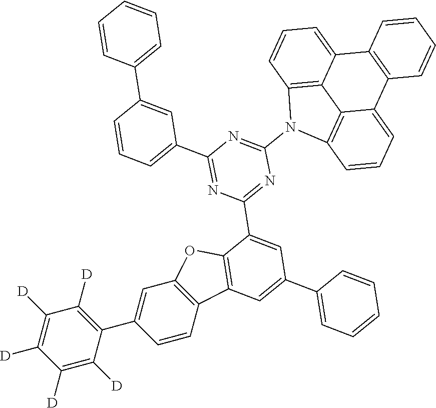 [SMILES: [2H]c1c([2H])c([2H])c(-c2ccc3c(c2)oc2c(-c4nc(-c5cccc(-c6ccccc6)c5)nc(-n5c6cccc7c8ccccc8c8cccc5c8c76)n4)cc(-c4ccccc4)cc23)c([2H])c1[2H]]

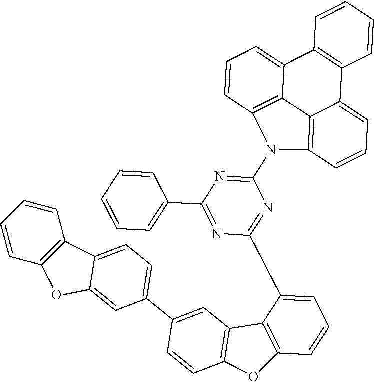 [SMILES: c1ccc(-c2nc(-c3cccc4oc5ccc(-c6ccc7c(c6)oc6ccccc67)cc5c34)nc(-n3c4cccc5c6ccccc6c6cccc3c6c54)n2)cc1]

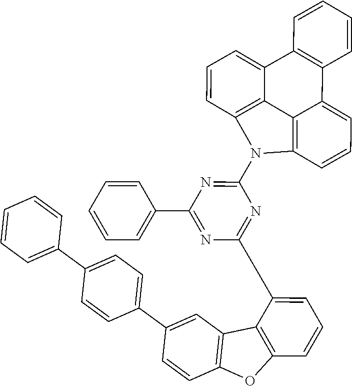 [SMILES: c1ccc(-c2ccc(-c3ccc4oc5cccc(-c6nc(-c7ccccc7)nc(-n7c8cccc9c%10ccccc%10c%10cccc7c%10c98)n6)c5c4c3)cc2)cc1]

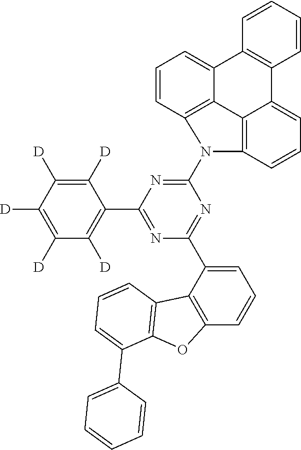 [SMILES: [2H]c1c([2H])c([2H])c(-c2nc(-c3cccc4oc5c(-c6ccccc6)cccc5c34)nc(-n3c4cccc5c6ccccc6c6cccc3c6c54)n2)c([2H])c1[2H]]

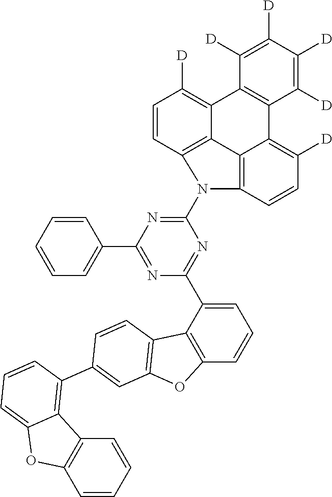 [SMILES: [2H]c1c([2H])c([2H])c2c(c1[2H])c1c([2H])ccc3c1c1c2c([2H])ccc1n3-c1nc(-c2ccccc2)nc(-c2cccc3oc4cc(-c5cccc6oc7ccccc7c56)ccc4c23)n1]

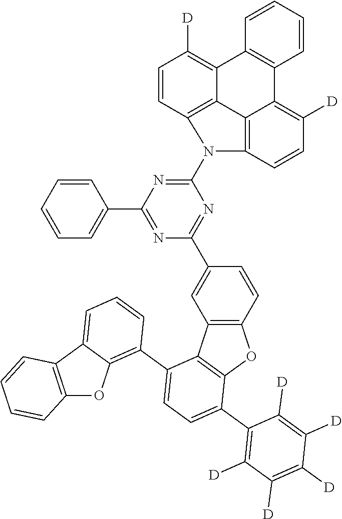 [SMILES: [2H]c1c([2H])c([2H])c(-c2ccc(-c3cccc4c3oc3ccccc34)c3c2oc2ccc(-c4nc(-c5ccccc5)nc(-n5c6ccc([2H])c7c8ccccc8c8c([2H])ccc5c8c76)n4)cc23)c([2H])c1[2H]]

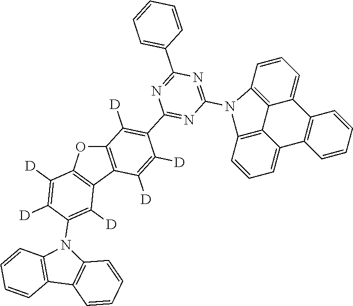 [SMILES: [2H]c1c(-n2c3ccccc3c3ccccc32)c([2H])c2c(oc3c([2H])c(-c4nc(-c5ccccc5)nc(-n5c6cccc7c8ccccc8c8cccc5c8c76)n4)c([2H])c([2H])c32)c1[2H]]

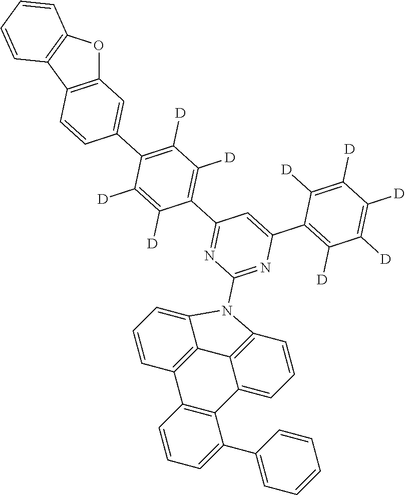 [SMILES: [2H]c1c([2H])c([2H])c(-c2cc(-c3c([2H])c([2H])c(-c4ccc5c(c4)oc4ccccc45)c([2H])c3[2H])nc(-n3c4cccc5c6cccc(-c7ccccc7)c6c6cccc3c6c54)n2)c([2H])c1[2H]]

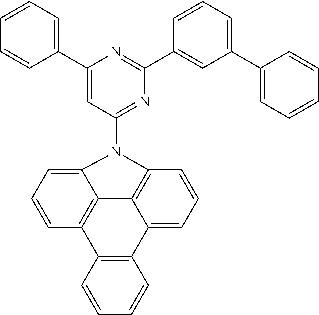 [SMILES: c1ccc(-c2cccc(-c3nc(-c4ccccc4)cc(-n4c5cccc6c7ccccc7c7cccc4c7c65)n3)c2)cc1]

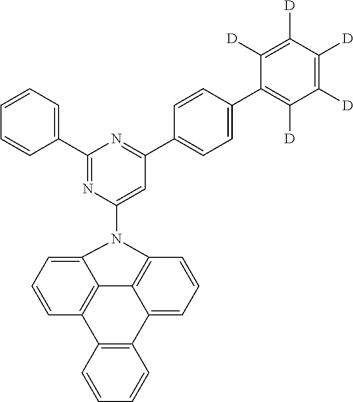 [SMILES: [2H]c1c([2H])c([2H])c(-c2ccc(-c3cc(-n4c5cccc6c7ccccc7c7cccc4c7c65)nc(-c4ccccc4)n3)cc2)c([2H])c1[2H]]

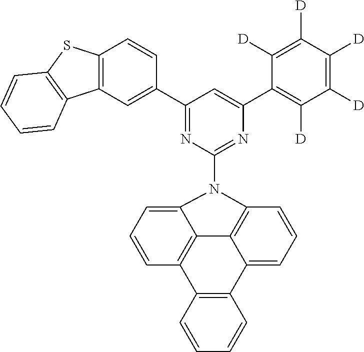 [SMILES: [2H]c1c([2H])c([2H])c(-c2cc(-c3ccc4sc5ccccc5c4c3)nc(-n3c4cccc5c6ccccc6c6cccc3c6c54)n2)c([2H])c1[2H]]

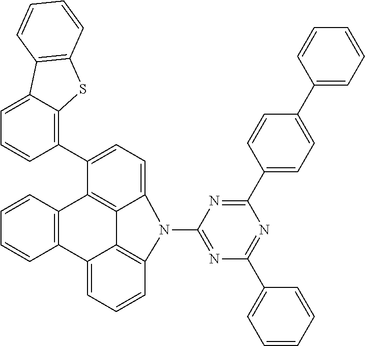 [SMILES: c1ccc(-c2ccc(-c3nc(-c4ccccc4)nc(-n4c5cccc6c7ccccc7c7c(-c8cccc9c8sc8ccccc89)ccc4c7c65)n3)cc2)cc1]

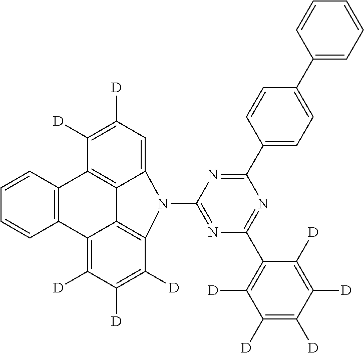 [SMILES: [2H]c1cc2c3c(c1[2H])c1ccccc1c1c([2H])c([2H])c([2H])c(c13)n2-c1nc(-c2ccc(-c3ccccc3)cc2)nc(-c2c([2H])c([2H])c([2H])c([2H])c2[2H])n1]

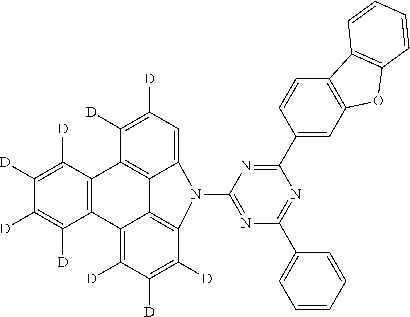 [SMILES: [2H]c1cc2c3c(c1[2H])c1c([2H])c([2H])c([2H])c([2H])c1c1c([2H])c([2H])c([2H])c(c13)n2-c1nc(-c2ccccc2)nc(-c2ccc3c(c2)oc2ccccc23)n1]

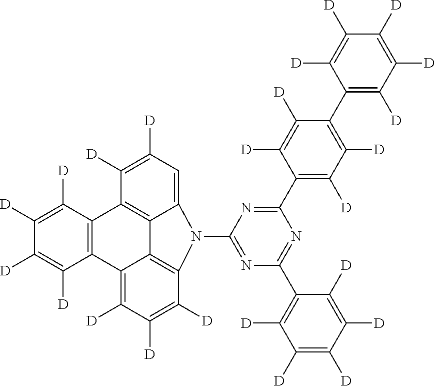 [SMILES: [2H]c1cc2c3c(c1[2H])c1c([2H])c([2H])c([2H])c([2H])c1c1c([2H])c([2H])c([2H])c(c13)n2-c1nc(-c2c([2H])c([2H])c([2H])c([2H])c2[2H])nc(-c2c([2H])c([2H])c(-c3c([2H])c([2H])c([2H])c([2H])c3[2H])c([2H])c2[2H])n1]

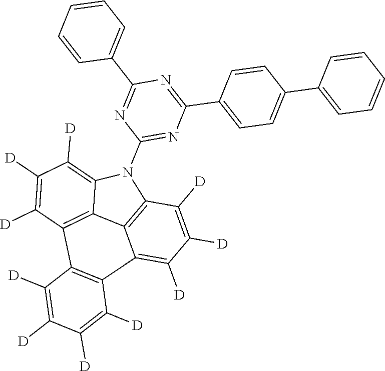 [SMILES: [2H]c1c([2H])c([2H])c2c(c1[2H])c1c([2H])c([2H])c([2H])c3c1c1c2c([2H])c([2H])c([2H])c1n3-c1nc(-c2ccccc2)nc(-c2ccc(-c3ccccc3)cc2)n1]

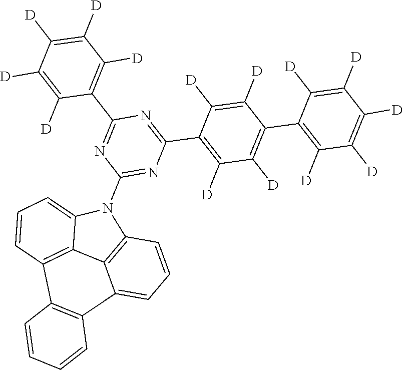 [SMILES: [2H]c1c([2H])c([2H])c(-c2nc(-c3c([2H])c([2H])c(-c4c([2H])c([2H])c([2H])c([2H])c4[2H])c([2H])c3[2H])nc(-n3c4cccc5c6ccccc6c6cccc3c6c54)n2)c([2H])c1[2H]]

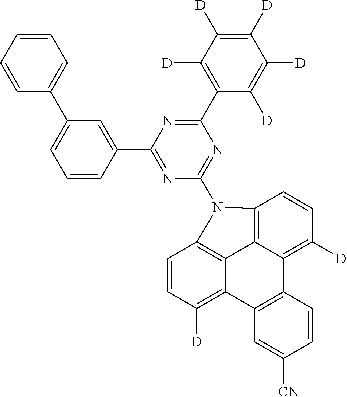 [SMILES: [2H]c1c([2H])c([2H])c(-c2nc(-c3cccc(-c4ccccc4)c3)nc(-n3c4ccc([2H])c5c6ccc(C#N)cc6c6c([2H])ccc3c6c54)n2)c([2H])c1[2H]]

Particularly suitable compounds of the formula (1) that are preferably used in combination with at least one compound of the formula (2) or of the formula (3) or of the formula (4) or of the formula (5) in the electroluminescent device of the invention are the compounds E1 to E15:

TABLE 2

| | |
|---|---|
| 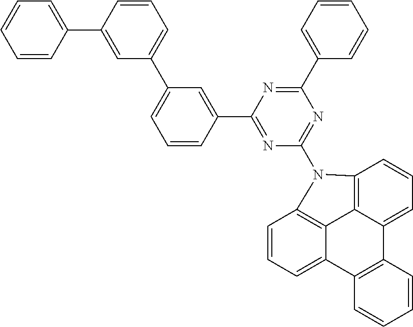 [SMILES: c1ccc(-c2cccc(-c3cccc(-c4nc(-c5ccccc5)nc(-n5c6cccc7c8ccccc8c8cccc5c8c76)n4)c3)c2)cc1] | E1 |
| 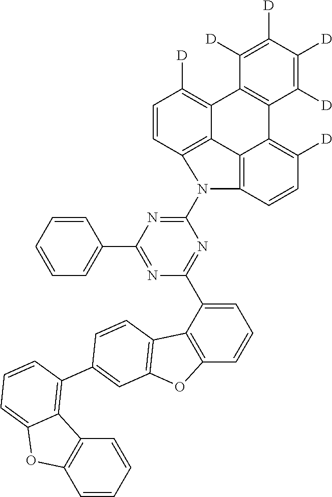 [SMILES: [2H]c1c([2H])c([2H])c2c(c1[2H])c1c([2H])ccc3c1c1c2c([2H])ccc1n3-c1nc(-c2ccccc2)nc(-c2cccc3oc4cc(-c5cccc6oc7ccccc7c56)ccc4c23)n1] | E2 |
| 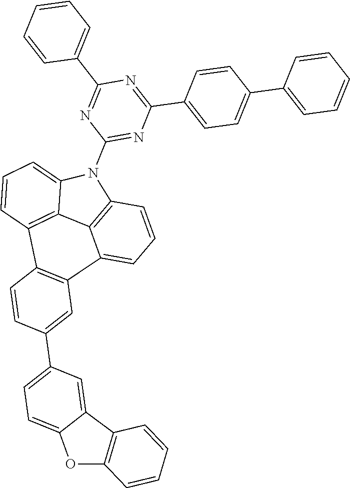 [SMILES: c1ccc(-c2ccc(-c3nc(-c4ccccc4)nc(-n4c5cccc6c7ccc(-c8ccc9oc%10ccccc%10c9c8)cc7c7cccc4c7c65)n3)cc2)cc1] | E3 |
| 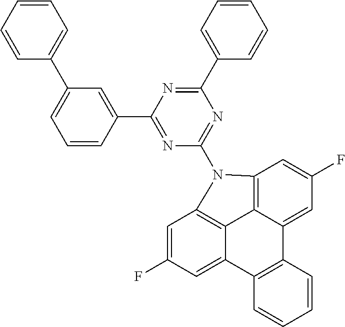 [SMILES: Fc1cc2c3ccccc3c3cc(F)cc4c3c2c(c1)n4-c1nc(-c2ccccc2)nc(-c2cccc(-c3ccccc3)c2)n1] | E4 |
| 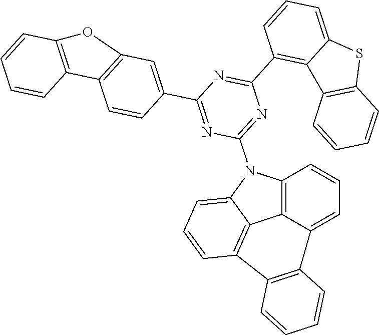 [SMILES: c1ccc2c(c1)oc1cc(-c3nc(-c4cccc5sc6ccccc6c45)nc(-n4c5cccc6c7ccccc7c7cccc4c7c65)n3)ccc12] | E5 |
| 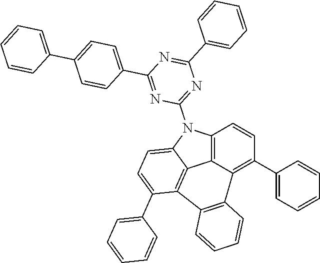 [SMILES: c1ccc(-c2ccc(-c3nc(-c4ccccc4)nc(-n4c5ccc(-c6ccccc6)c6c7ccccc7c7c(-c8ccccc8)ccc4c7c65)n3)cc2)cc1] | E6 |
| 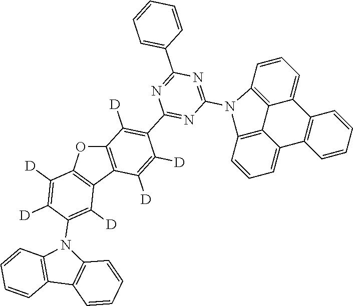 [SMILES: [2H]c1c(-n2c3ccccc3c3ccccc32)c([2H])c2c(oc3c([2H])c(-c4nc(-c5ccccc5)nc(-n5c6cccc7c8ccccc8c8cccc5c8c76)n4)c([2H])c([2H])c32)c1[2H]] | E7 |
| 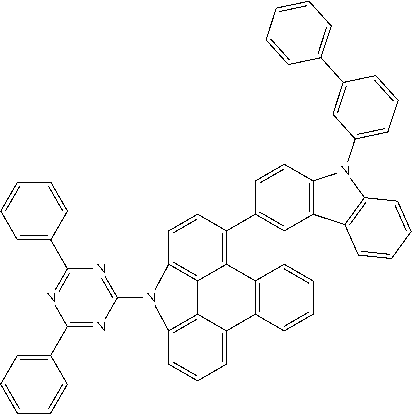 [SMILES: c1ccc(-c2cccc(-n3c4ccccc4c4cc(-c5ccc6c7c5c5ccccc5c5cccc(c57)n6-c5nc(-c6ccccc6)nc(-c6ccccc6)n5)ccc43)c2)cc1] | E8 |
| 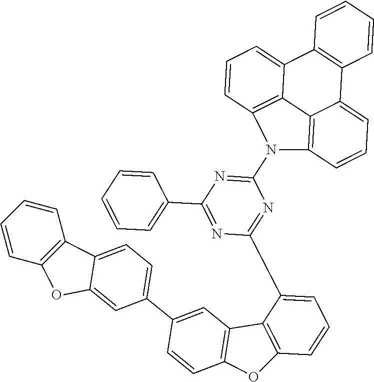 [SMILES: c1ccc(-c2nc(-c3cccc4oc5ccc(-c6ccc7c(c6)oc6ccccc67)cc5c34)nc(-n3c4cccc5c6ccccc6c6cccc3c6c54)n2)cc1] | E9 |
| 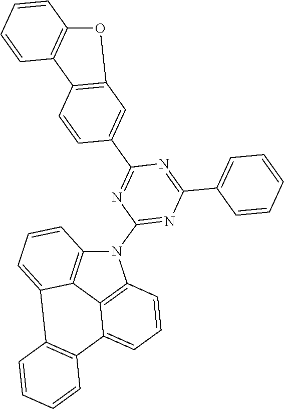 [SMILES: c1ccc(-c2nc(-c3ccc4c(c3)oc3ccccc34)nc(-n3c4cccc5c6ccccc6c6cccc3c6c54)n2)cc1] | E10 |
| 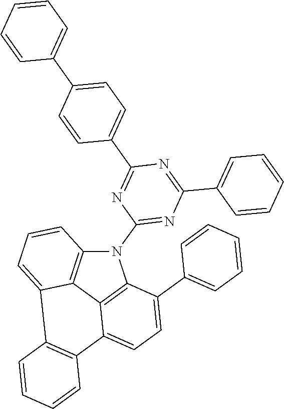 [SMILES: c1ccc(-c2ccc(-c3nc(-c4ccccc4)nc(-n4c5cccc6c7ccccc7c7ccc(-c8ccccc8)c4c7c65)n3)cc2)cc1] | E11 |
| 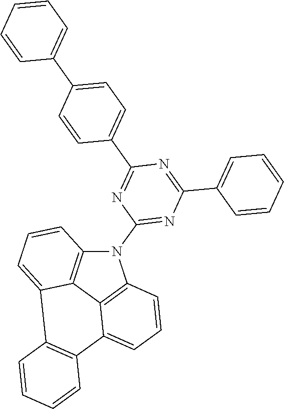 [SMILES: c1ccc(-c2ccc(-c3nc(-c4ccccc4)nc(-n4c5cccc6c7ccccc7c7cccc4c7c65)n3)cc2)cc1] | E12 |
| 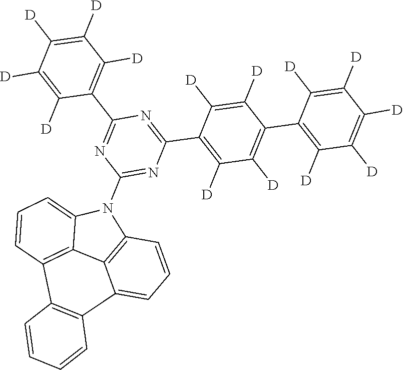 [SMILES: [2H]c1c([2H])c([2H])c(-c2nc(-c3c([2H])c([2H])c(-c4c([2H])c([2H])c([2H])c([2H])c4[2H])c([2H])c3[2H])nc(-n3c4cccc5c6ccccc6c6cccc3c6c54)n2)c([2H])c1[2H]] | E13 |
| 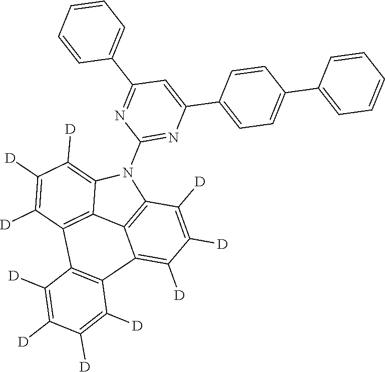 [SMILES: [2H]c1c([2H])c([2H])c2c(c1[2H])c1c([2H])c([2H])c([2H])c3c1c1c2c([2H])c([2H])c([2H])c1n3-c1nc(-c2ccccc2)cc(-c2ccc(-c3ccccc3)cc2)n1] | E14 |
| 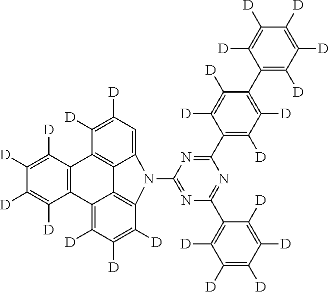 [SMILES: [2H]c1cc2c3c(c1[2H])c1c([2H])c([2H])c([2H])c([2H])c1c1c([2H])c([2H])c([2H])c(c13)n2-c1nc(-c2c([2H])c([2H])c([2H])c([2H])c2[2H])nc(-c2c([2H])c([2H])c(-c3c([2H])c([2H])c([2H])c([2H])c3[2H])c([2H])c2[2H])n1] | E15 |
| 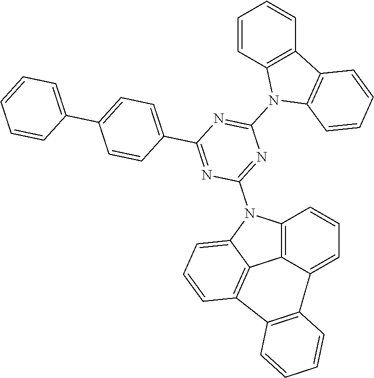 [SMILES: c1ccc(-c2ccc(-c3nc(-n4c5ccccc5c5ccccc54)nc(-n4c5cccc6c7ccccc7c7cccc4c7c65)n3)cc2)cc1] | E16 |
| 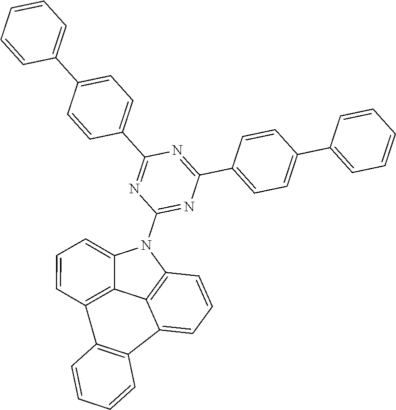 [SMILES: c1ccc(-c2ccc(-c3nc(-c4ccc(-c5ccccc5)cc4)nc(-n4c5cccc6c7ccccc7c7cccc4c7c65)n3)cc2)cc1] | E17 |
| 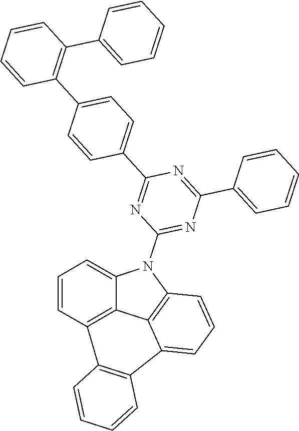 [SMILES: c1ccc(-c2nc(-c3ccc(-c4ccccc4-c4ccccc4)cc3)nc(-n3c4cccc5c6ccccc6c6cccc3c6c54)n2)cc1] | E18 |

The preparation of the compounds of the formula (1) is known to those skilled in the art. The preparation of the compounds of the formula (1) or of the preferred compounds of the formulae (1-1) and (1-2) is described in WO2012/048781 inter alia.

Compounds of the formula (2) or of the formula (3) or of the formula (4) are depicted below:

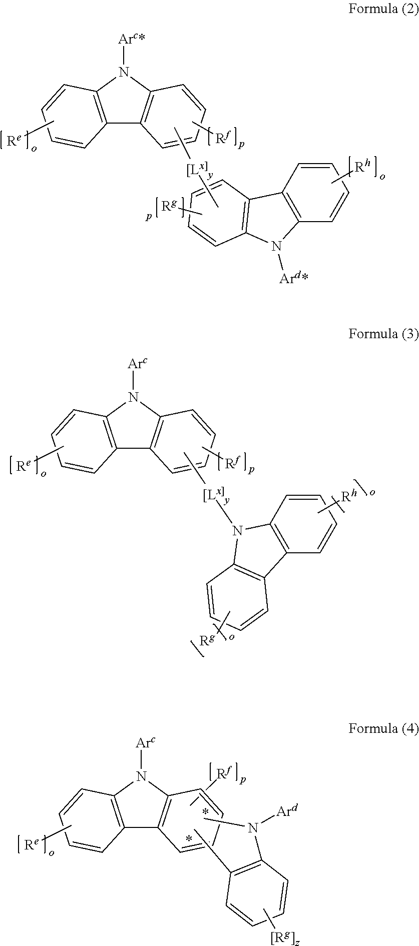

where the symbols used have the definitions given above.

Preferred compounds of the formula (2) or of the formula (3) are compounds of the formulae (2-1) to (2-4) or (3-1) to (3-4), more preferably compounds of the formulae (2-2) and (2-4) or (3-2) and (3-4), especially compounds of the formulae (2-2) and (2-4):

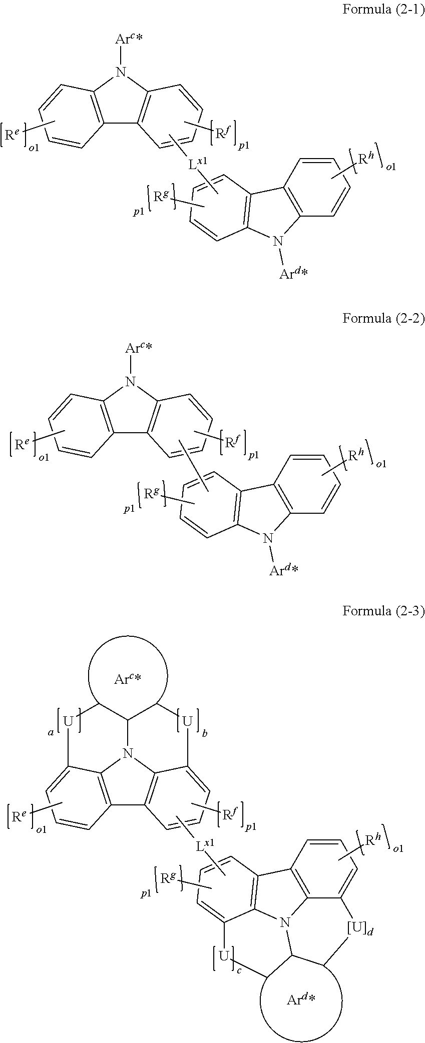

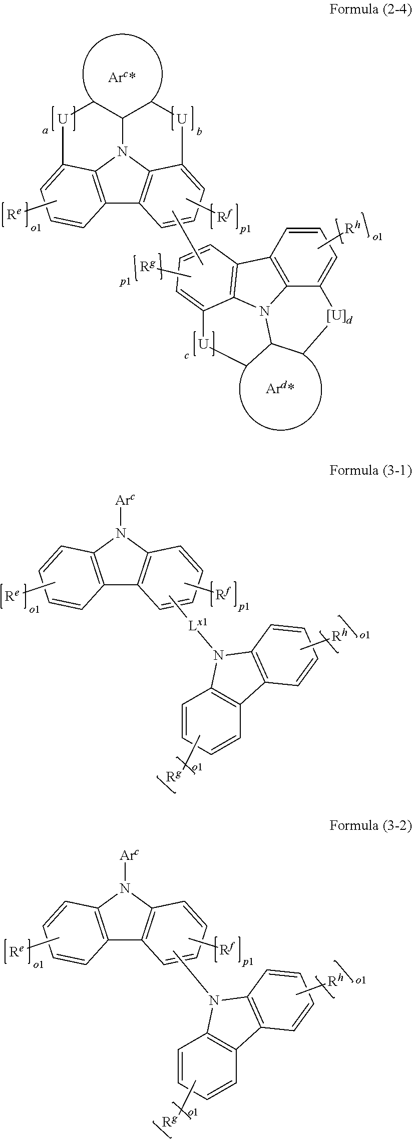

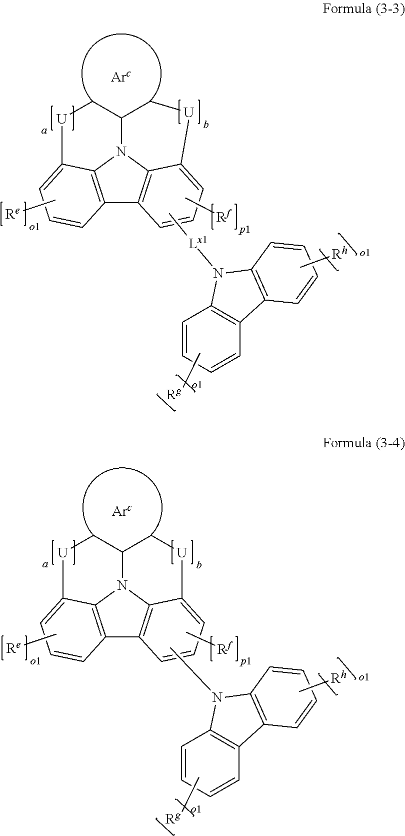

where the symbols used have the definitions given above, L^{x1} in the formulae (2-1), (2-3), (3-1) and (3-3) denotes an aromatic ring system having 6 to 40 aromatic ring atoms or a heteroaromatic ring system having 5 to 40 aromatic ring atoms, which may be substituted by one or more R¹ radicals, where o1 is the same or different at each instance and is independently 0, 1, 2, 3 or 4, p1 is the same or different at each instance and is independently 0, 1, 2 or 3.

In a preferred embodiment of the compounds of the formulae (2-3) and (2-4), and (3-3) and (3-4), the sum total of the indices a+b and/or the sum total of the indices c+d is independently equal to 1; most preferably, the sum total of the indices a+b and the sum total of the indices c+d are each independently equal to 1.

In preferred embodiments of the compounds of the formulae (2-3), (2-4), (3-3) and (3-4), the indices are as follows: a=1 and b, c and d=0; or a, b, c=0 and d=1, or a, d=1 and b, c=0.

In a preferred embodiment of the compounds of the formula (2) and of the formula (3), the indices 01 and p1 are the same or different and are independently 0, 1 or 2, more preferably 0 or 1; in particular, all indices are 0. If the R^{e}, R^{f}, R^{g} and R^{h} radicals are D, it is preferable that the indices assume the maximum possible number, i.e. o1=4, p1=3. The same applies to compounds of the formula (4) with the indices o, p and z, and the R^{e}, R^{f} and R^{h} radicals. Preferably, o and z are the same or different at each instance and are independently 0, 1 or 2, more preferably 0 or 1, especially 0. p is preferably 0 or 1, especially 0. If R^{e}, R^{f} and R^{g} are D, o and z are preferably 4 and p is preferably 2.

In a further preferred embodiment of the compounds of the formula (2) and of the formula (3), at least one of the carbazoles is bonded to the second carbazole via the 3 position.

If, in compounds of the formulae (2) and (2-1) to (2-4) or of the formulae (3) and (3-1) to (3-4) or of the formula (4), o or 01 and/or p or p1 and/or z is greater than 0, the respective substituent R^{e}, R^{f}, R^{g} and R^{h} is the same or different at each instance and is preferably selected from the group consisting of D, F, an alkyl group having 1 to 10 carbon atoms or an aromatic or heteroaromatic ring system which has 6 to 24 aromatic ring atoms and may be substituted by one or more R¹ radicals. The aromatic or heteroaromatic ring system having 6 to 24 aromatic ring atoms in these R^{e}, R^{f}, R^{g} and R^{h} radicals is preferably derived from benzene, dibenzofuran, dibenzothiophene, 9-phenylcarbazole, biphenyl and terphenyl, which may be substituted by one or more R¹ radicals. The preferred position of the substituents is position 1, 2, 3 or 4 or the combinations of positions 1 and 4 and 1 and 3, more preferably 1 and 3, 2 or 3, most preferably 3, where R^{e}, R^{f}, R^{g} and R^{h} have one of the preferred definitions given above and o, o1, p, p1 and z are each independently greater than 0. Particularly preferred substituents R^{e}, R^{f}, R^{g} and R^{h} are carbazol-9-yl, biphenyl, terphenyl, triphenylenyl and dibenzofuranyl.

Ar′ in N(Ar′)₂ is preferably derived from benzene, dibenzofuran, fluorene, spirobifluorene, dibenzothiophene, 9-phenylcarbazole, biphenyl and terphenyl which may be substituted by one or more substituents R¹, or combinations of these groups. Ar′ here is preferably unsubstituted.

In compounds of the formulae (2) and (2-1) to (2-4), Ar^{c*} and Ar^{d*} are preferably each independently an aromatic ring system having 6 to 30 aromatic ring atoms or a heteroaryl group selected from the group consisting of dibenzofuran, dibenzothiophene, carbazole, triphenyleno[1,2-bcd]thiophene, substituted naphtho[1,2,3,4, def]carbazole, phenoxazine, phenothiazine, indolo[3,2,1-jk]carbazole, biscarbazole, benzcarbazole, indenocarbazole, indolocarbazole, benzofurocarbazole, benzothioenocarbazole, each of which may be substituted by one or more R^{d} radicals, where, independently of one another, an R^{f} radical or an R^{e} radical together with an Ar^{c*} radical or an R^{g} radical or an R^{h} radical together with an Ar^{d*} radical may form a monocyclic or polycyclic, aliphatic, aromatic or heteroaromatic ring, or a group of the formula (6a) or (6b), where the dashed bond represents the bond to the nitrogen atom in the formula (2), in the formula (3) or in the formula (4);

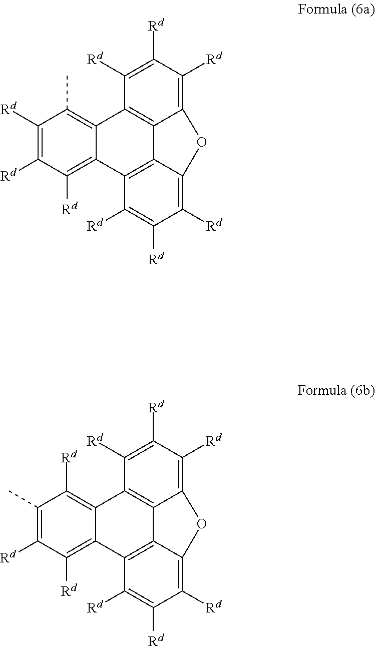

Ar^{c*} and Ar^{d*} are more preferably derived from benzene, dibenzofuran, fluorene, spirobifluorene, dibenzothiophene, 9-phenylcarbazole, biphenyl, naphthyl, triphenylene and terphenyl, which may be substituted by one or more substituents R^{d}, or combinations of these groups, where R^{d} has the definition given above.

If Ar^{c*} and Ar^{d*} are a heteroaryl group that may be substituted by one or more of the substituents R^{d}, particular preference is given to electron-rich ring systems, where the optionally R^{d}-substituted heteroaryl group preferably contains just one nitrogen atom in its entirety or the optionally R^{d}-substituted heteroaryl group contains one or more oxygen and/or sulfur atoms in its entirety.

In compounds of the formulae (3), (3-1) to (3-4) and of the formula (4), as described above, Ar^{c} and Ar^{d} are preferably each independently an aromatic ring system having 6 to 30 aromatic ring atoms or a heteroaromatic ring system having 6 to 30 aromatic ring atoms, which may be substituted by one or more R^{d} radicals. Ar^{c} and Ar^{d} are preferably derived from benzene, dibenzofuran, fluorene, spirobifluorene, dibenzothiophene, 9-phenylcarbazole, biphenyl, naphthyl, triphenylenyl and terphenyl, which may be substituted by one or more substituents R^{d}, or combinations of these groups, where R^{d} has the definition given above.

If Ar^{c} and Ar^{d} are a heteroaromatic ring system which has 6 to 40 carbon atoms and may be substituted by one or more of the substituents R^{d}, particular preference is given to electron-rich ring systems, where the optionally R^{d}-substituted ring system preferably contains just one nitrogen atom in its entirety or the optionally R^{d}-substituted ring system contains one or more oxygen and/or sulfur atoms in its entirety.

In the compounds of the formulae (2), (2-1), (2-3), (3), (3-1) and (3-3), the linker L^{x} or L^{x1}, if it is not a single bond, is preferably selected from the linkers L-2.1 to L-2.33:

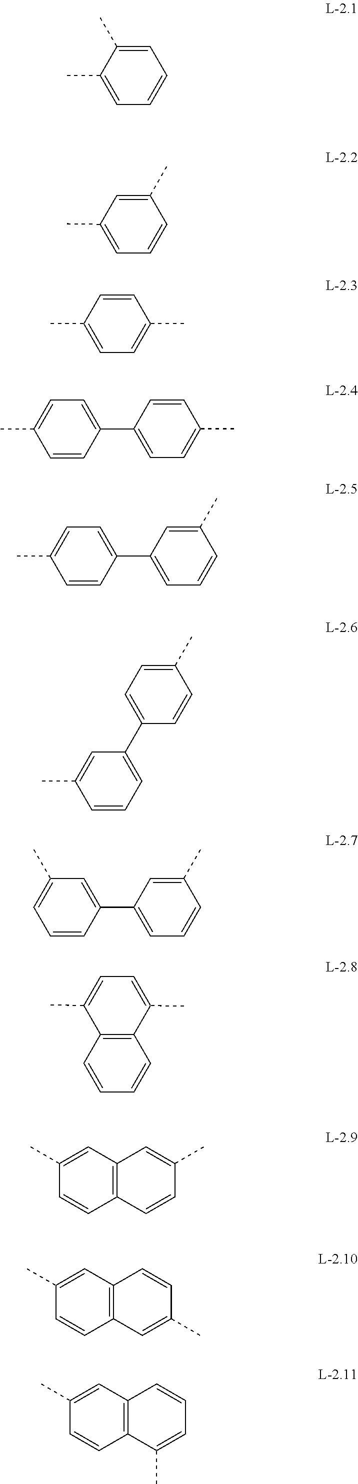

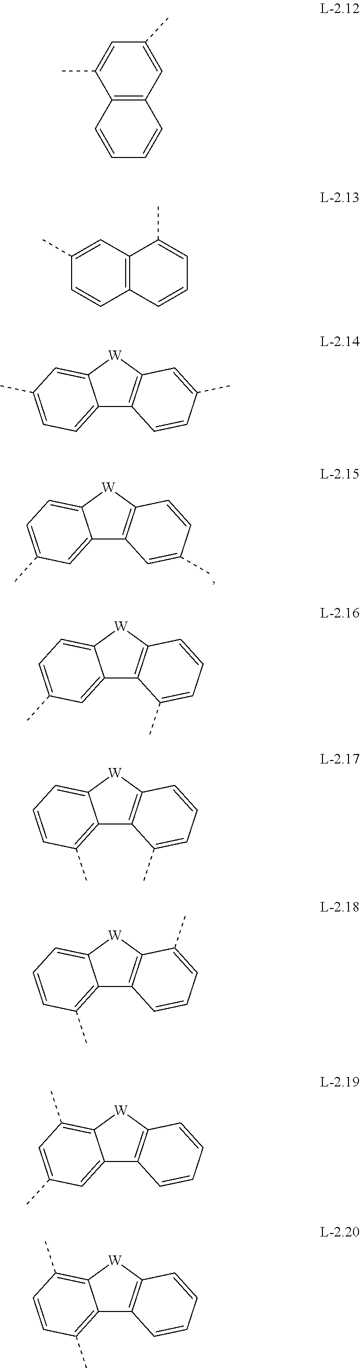

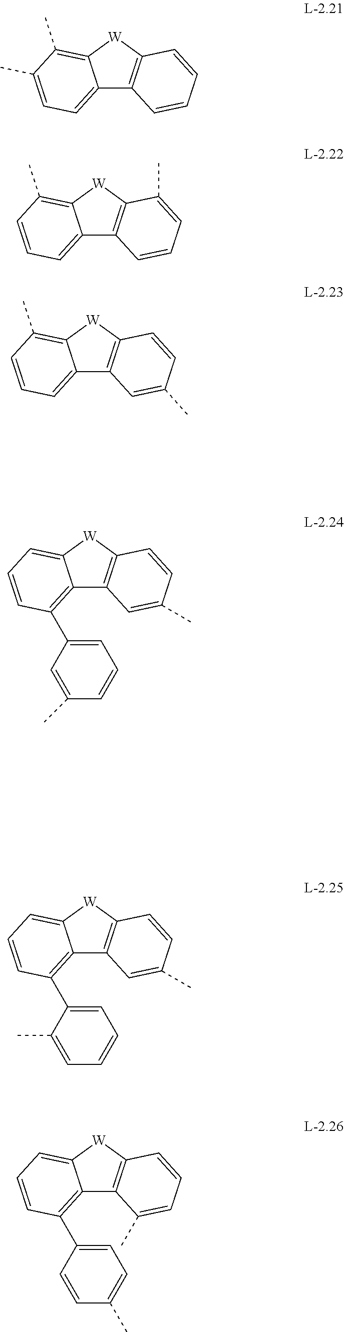

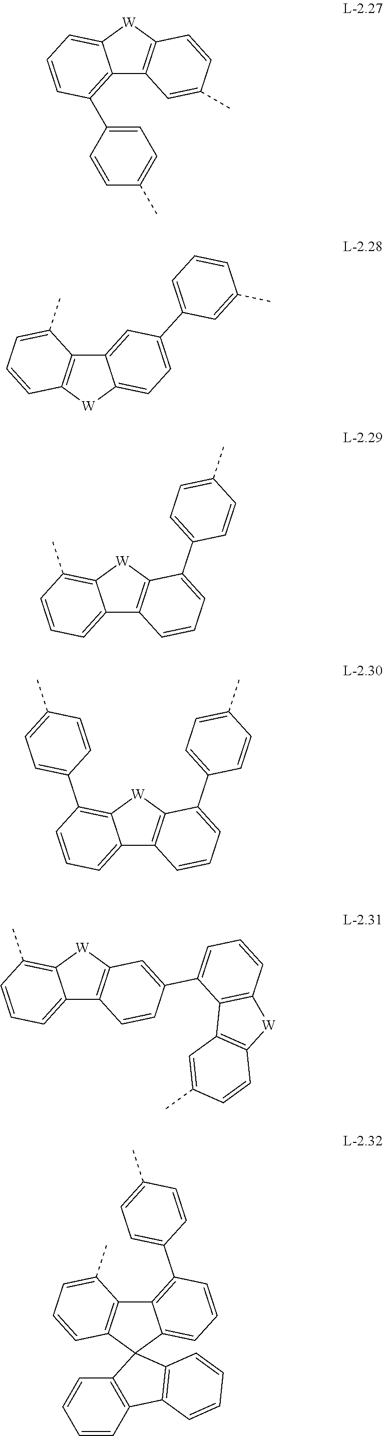

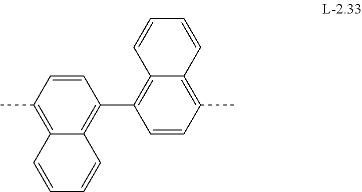

where W is NAr′, O, S or C(CH₃)₂, Ar′ has the definition given above, the linkers L-2.1 to L-2.33 may be substituted by one or more R¹ radicals and the dashed lines denote the attachment to the carbazoles.

The linkers L-2.1 to L-2.33 are preferably unsubstituted, where the hydrogen atoms may be wholly or partly replaced by D or may be replaced by a phenyl.

Preferred linkers for L^{x} and L^{x1} are selected from the structures L-2.1 to L-2.33 in which W is defined as O, S or NAr′, more preferably as O or NAr′.

In a particularly preferred embodiment of the invention, the abovementioned preferences for linkers and indices occur simultaneously.

Preferred embodiments of the formulae (2-1) and (2-2) or (3-1) and (3-2) or of the formula (4) are the compounds of the formulae (2-1-1) to (2-1-3), (3-1-1) to (3-1-3), (2-2-1) to (2-2-16), (3-2-1) to (3-2-4) and (4-1) to (4-6), more preferably compounds of the formulae (2-1-1), (2-1-3), (2-2-3), (2-2-4), (2-2-15), (5-2-16), (4-2) and (4-4), especially compounds of the formulae (2-2-4), (4-2) and (4-4). It is also possible here for the hydrogen atoms to be wholly or partly replaced by deuterium. The compounds are more preferably fully or partly deuterated, especially fully deuterated.

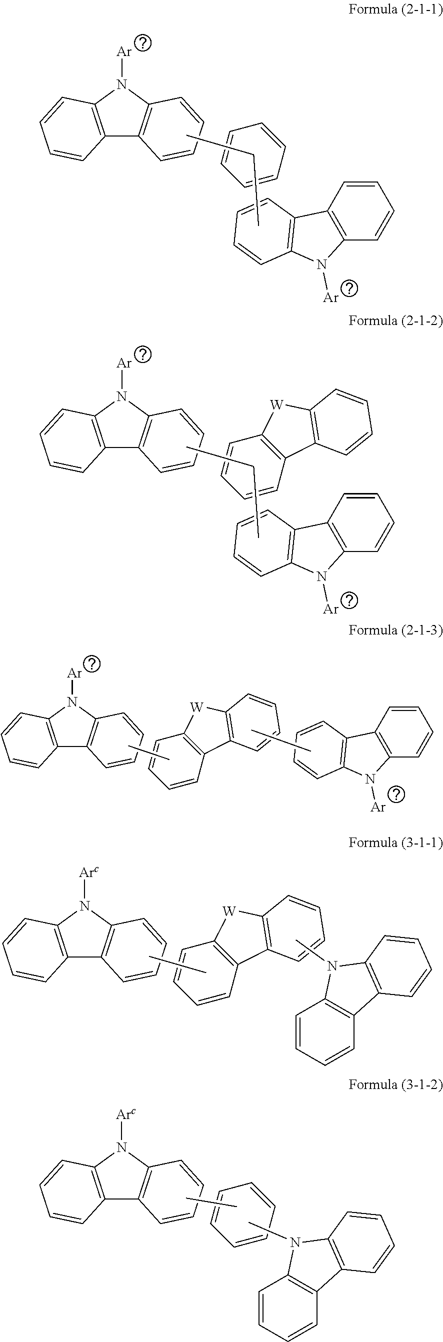

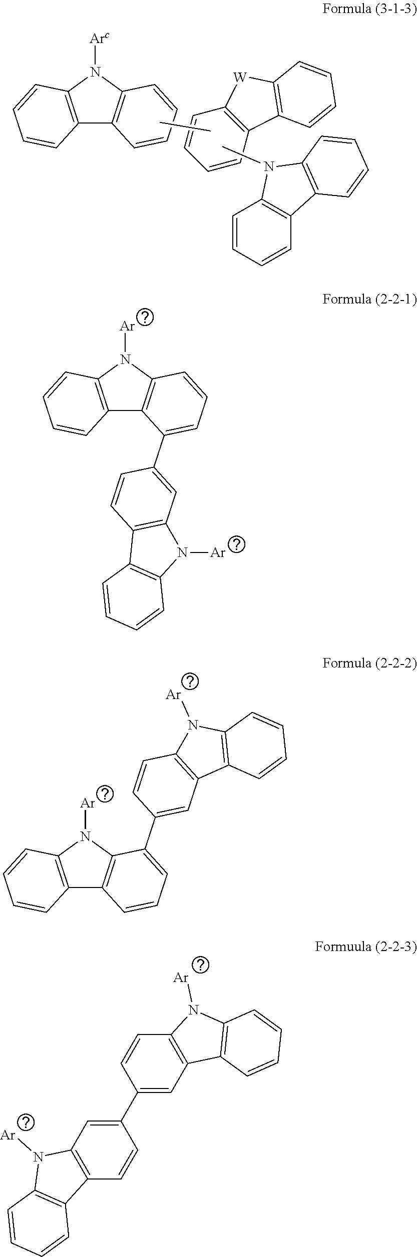

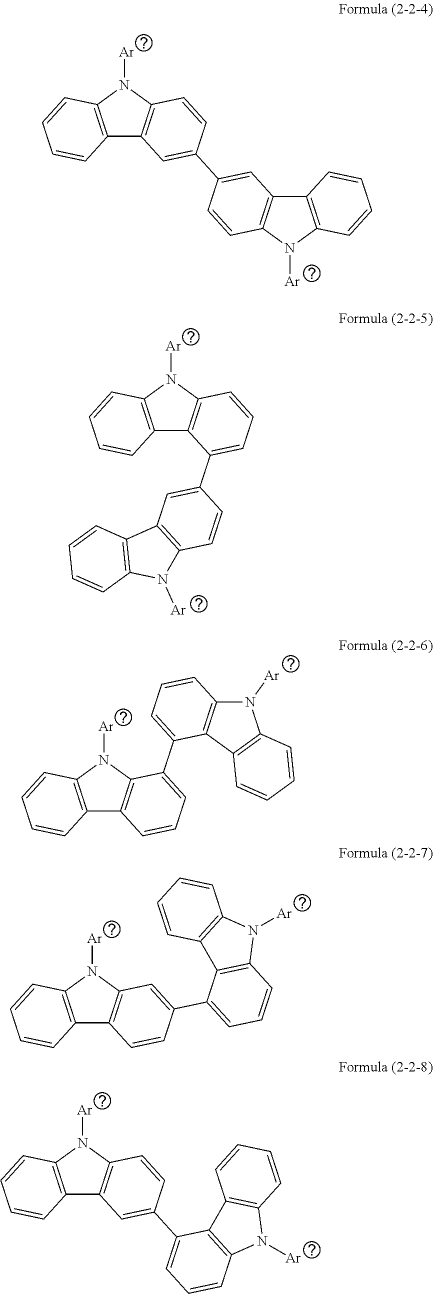

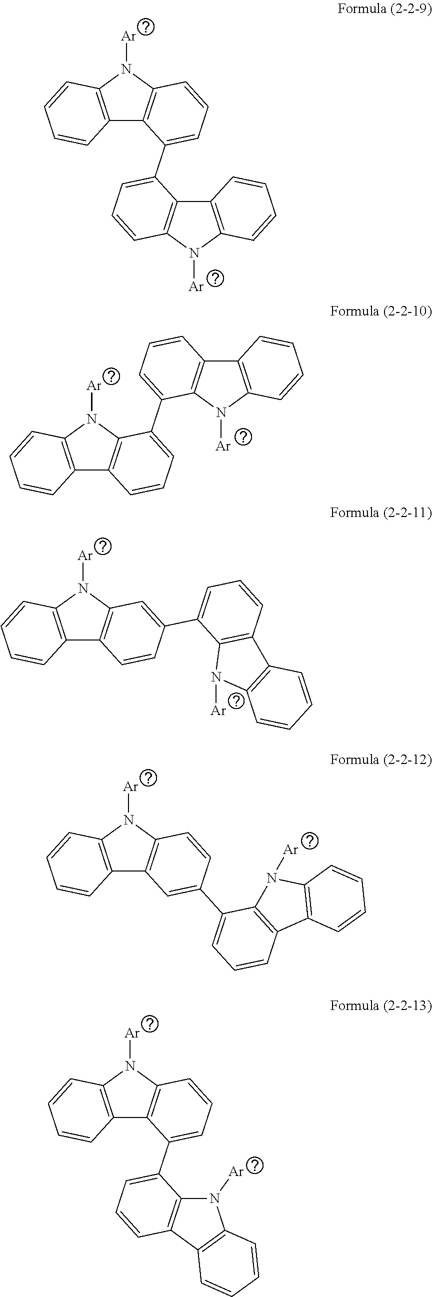

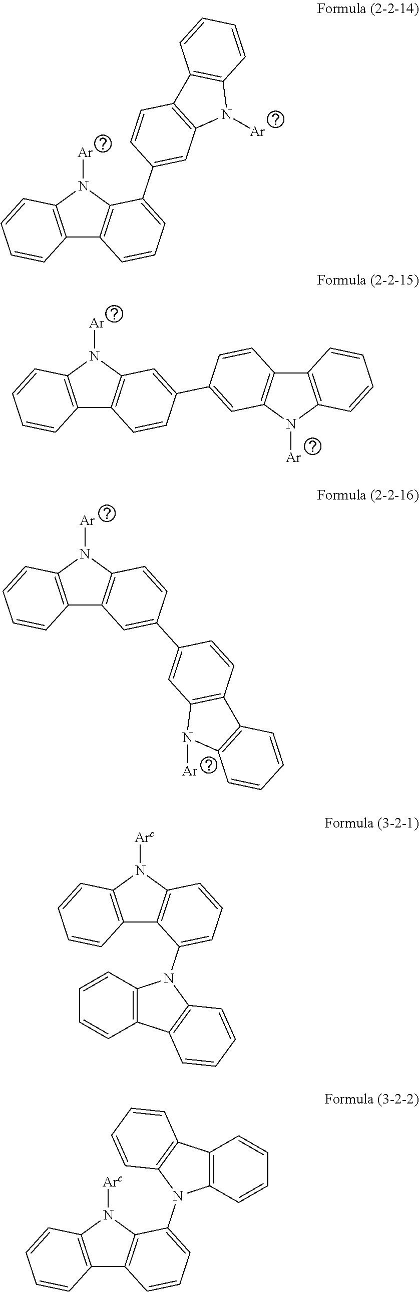

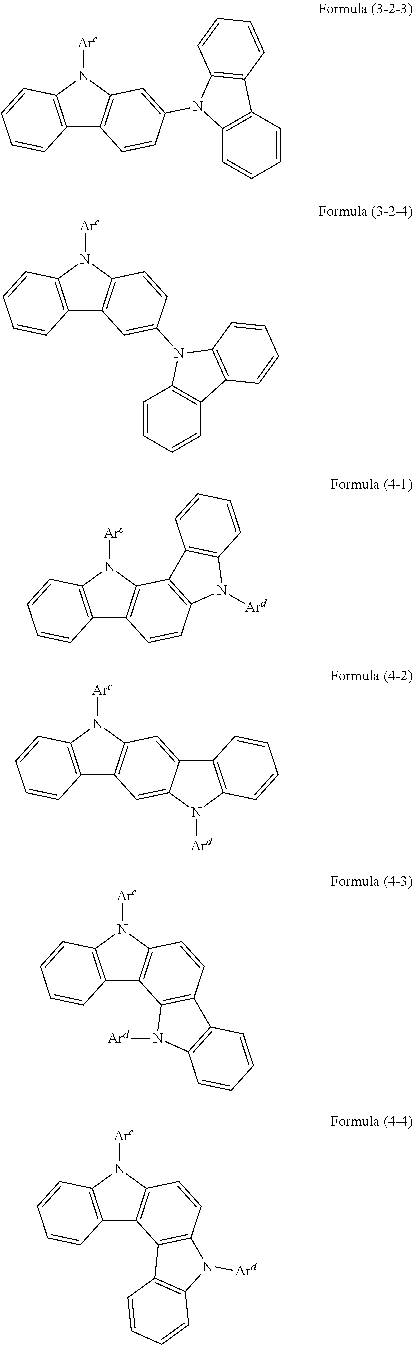

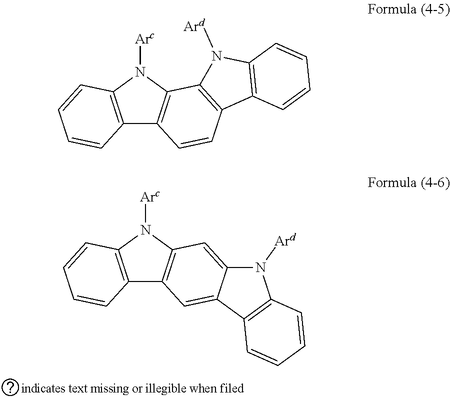

where the symbols used have the definitions given above.

Preferred embodiments of the formulae (2-3) and (2-4) or (3-3) and (3-4) are the compounds of the formulae (2-3-1) to (2-3-5), (3-3-1) to (3-3-3), (2-4-1) to (2-4-5) and (3-4-1) to (3-4-2), more preferably compounds of the formulae (2-3-5), (2-4-1), (2-4-2) (2-4-3), (2-4-4), (2-4-5) and (3-4-1), especially compounds of the formulae (2-4-1) and (2-4-2). It is also possible here for the hydrogen atoms to be wholly or partly replaced by deuterium. The compounds are more preferably fully or partly deuterated, especially fully deuterated.

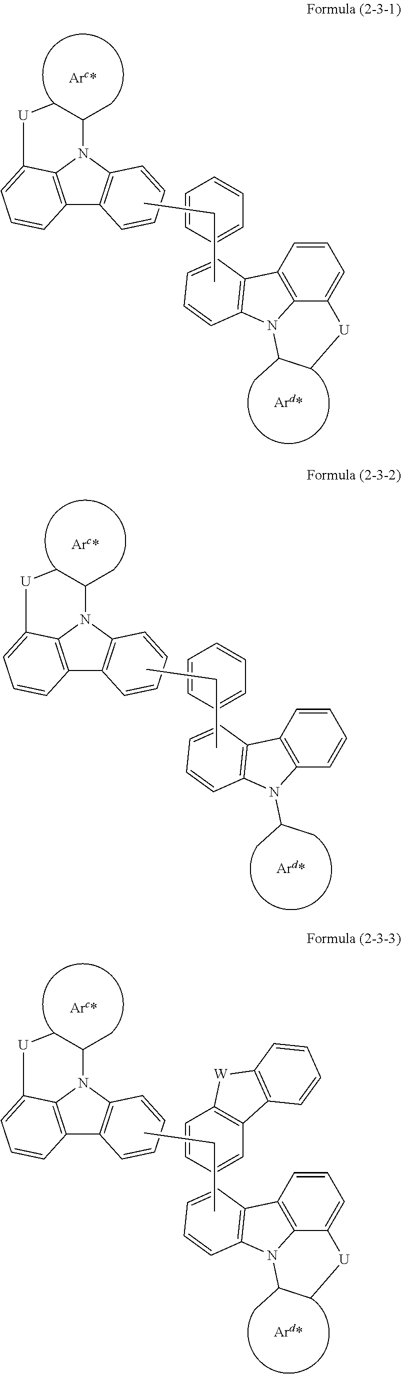

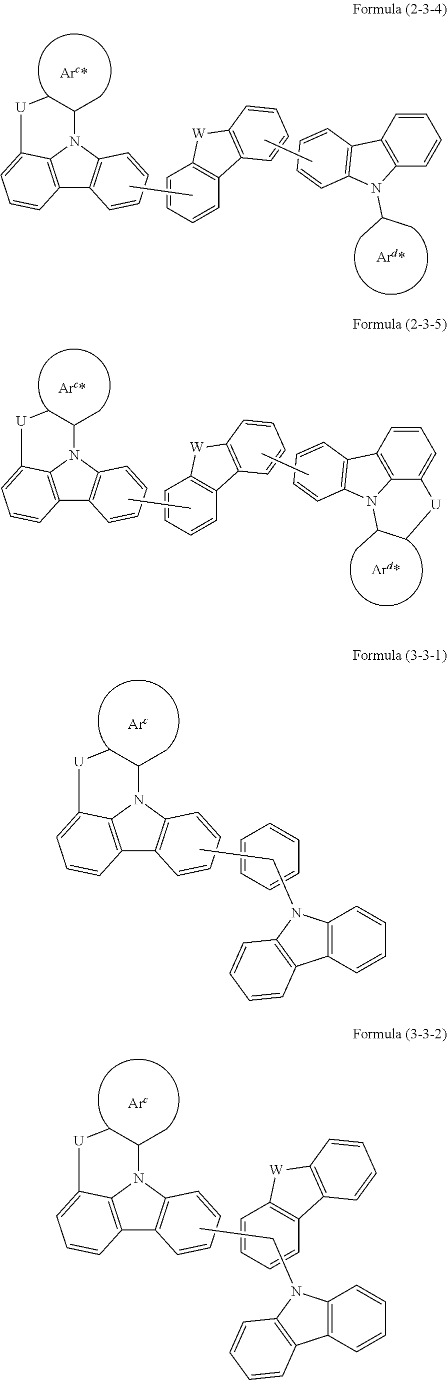

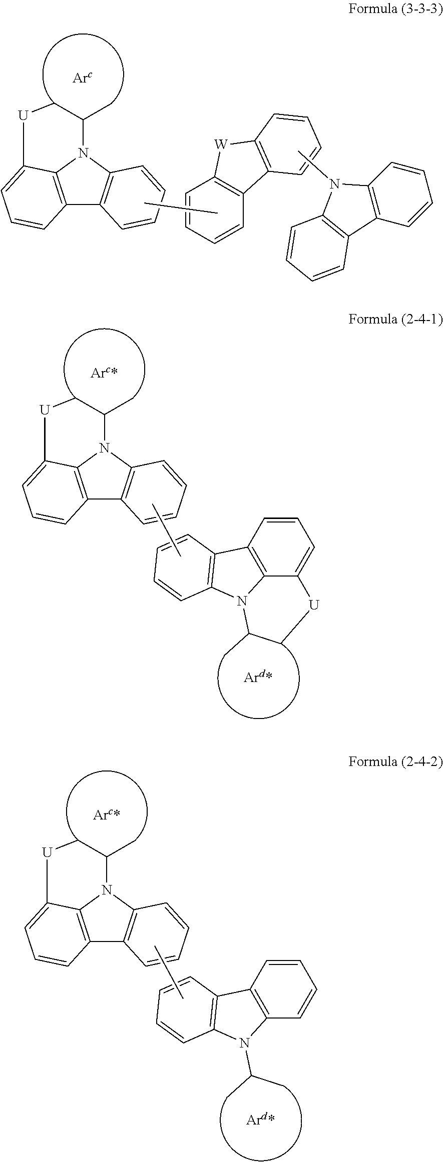

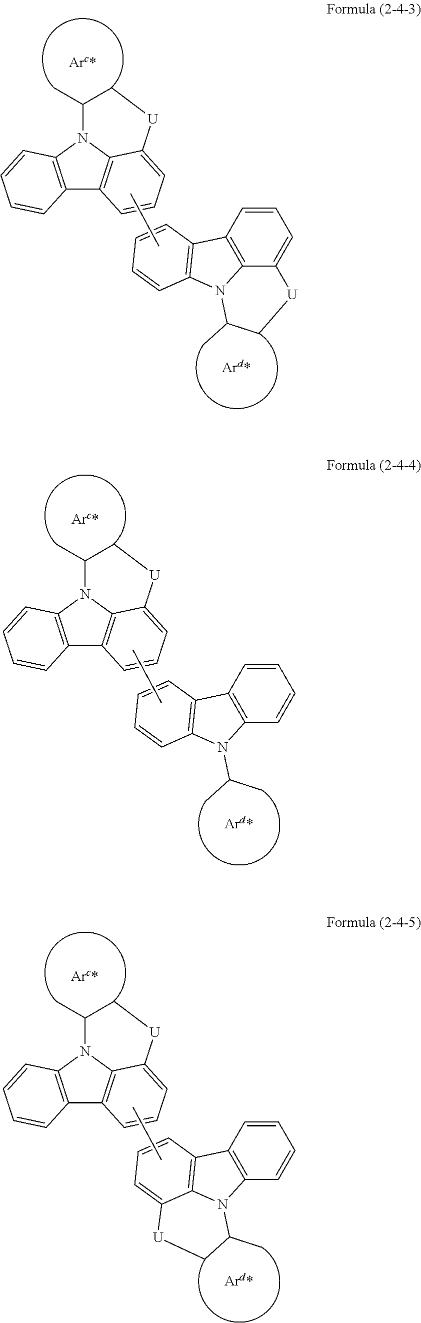

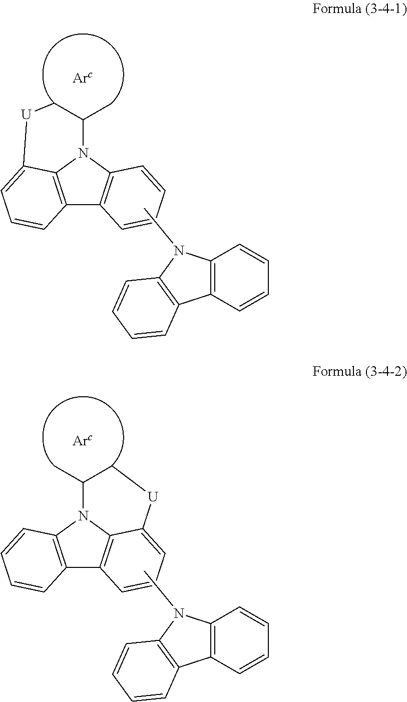

where the symbols used have the definitions given above.

There follows a description of the host material of the formula (5) and preferred embodiments thereof.

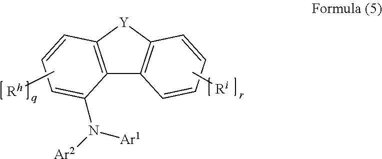

In a preferred embodiment of the compounds of the formula (5), Ar¹ and Ar² are independently selected from the following groups R2-1 to R2-222 from table 3:

TABLE 3

| | |
|---|---|
| 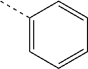 | R2-1 |
| 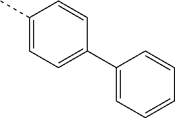 | R2-2 |
| 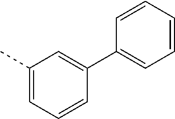 | R2-3 |
| 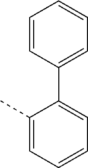 | R2-4 |
| 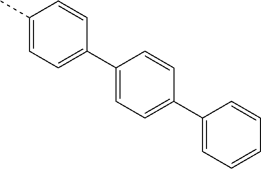 | R2-5 |
| 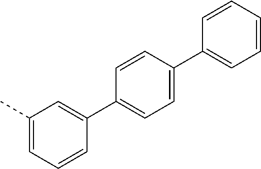 | R2-6 |
| 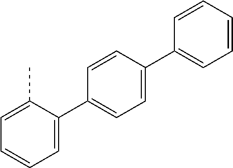 | R2-7 |
| 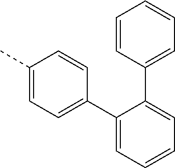 | R2-8 |
| 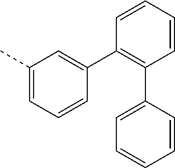 | R2-9 |
| 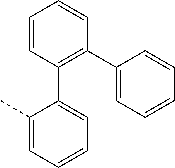 | R2-10 |
| 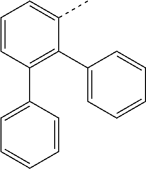 | R2-11 |
| 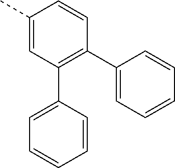 | R2-12 |
| 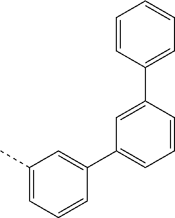 | R2-13 |
| 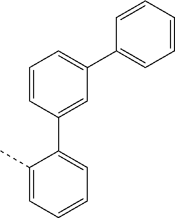 | R2-14 |
| 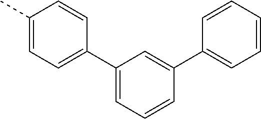 | R2-15 |
| 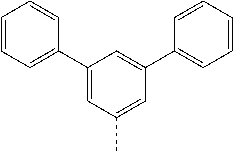 | R2-16 |
| 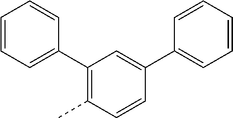 | R2-17 |
| 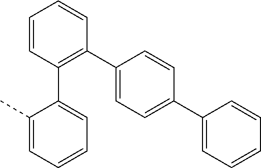 | R2-18 |
| 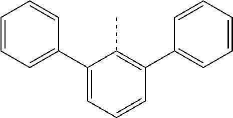 | R2-19 |
| 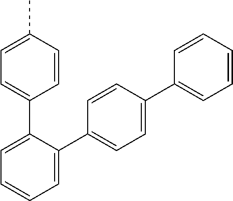 | R2-20 |
| 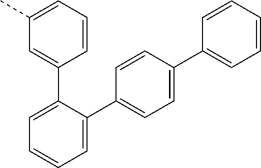 | R2-21 |
| 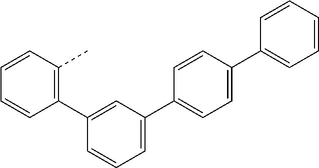 | R2-22 |
| 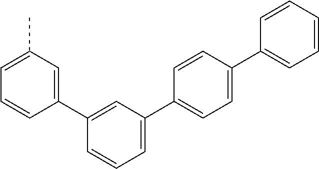 | R2-23 |
| 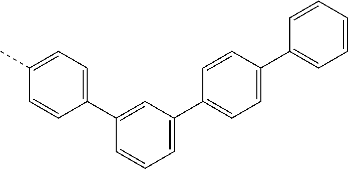 | R2-24 |
| 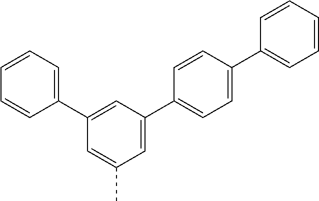 | R2-25 |
| 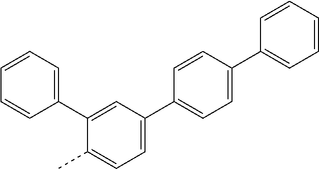 | R2-26 |
| 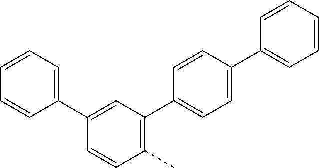 | R2-27 |
| 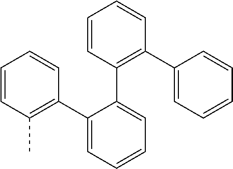 | R2-28 |
| 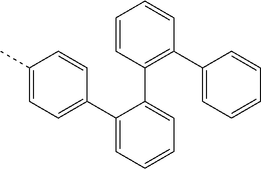 | R2-29 |
| 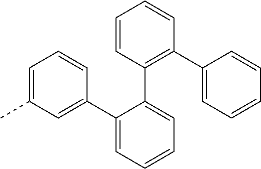 | R2-30 |
| 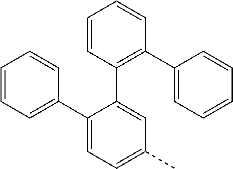 | R2-31 |
| 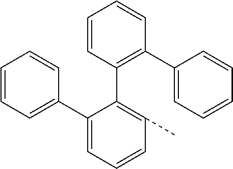 | R2-32 |
| 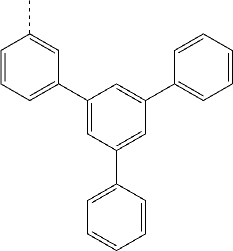 | R2-33 |
| 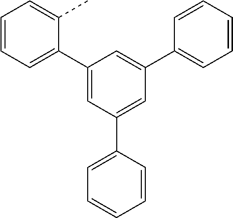 | R2-34 |
| 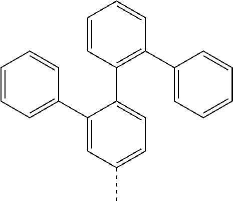 | R2-35 |
| 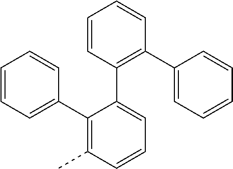 | R2-36 |
| 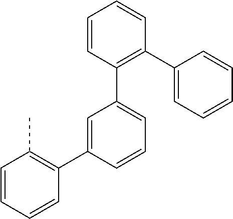 | R2-37 |
| 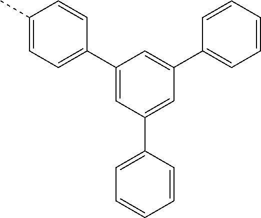 | R2-38 |
| 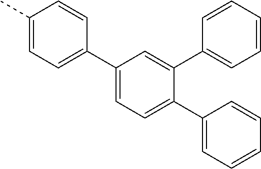 | R2-39 |
| 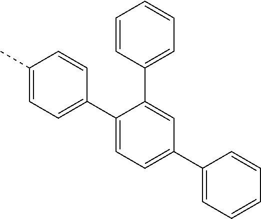 | R2-40 |
| 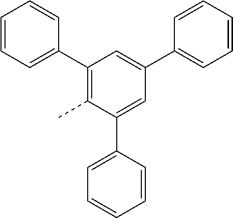 | R2-41 |
| 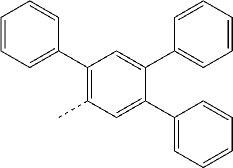 | R2-42 |
| 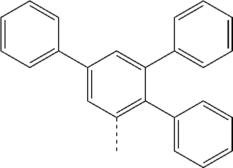 | R2-43 |
| 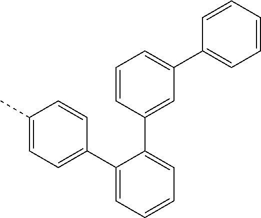 | R2-44 |
| 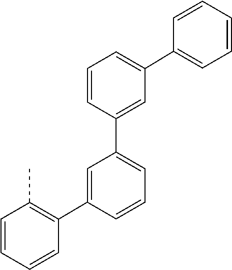 | R2-45 |
| 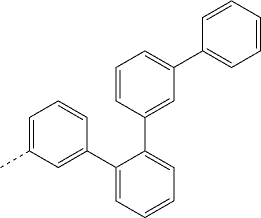 | R2-46 |
| 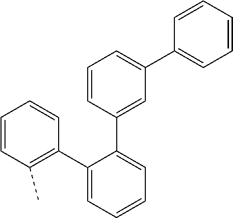 | R2-47 |
| 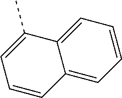 | R2-48 |
| 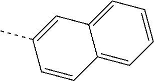 | R2-49 |
| 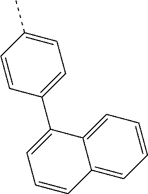 | R2-50 |
| 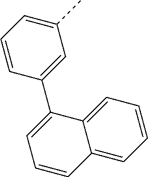 | R2-51 |
| 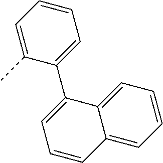 | R2-52 |
| 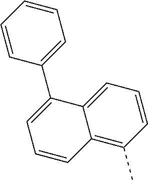 | R2-53 |
| 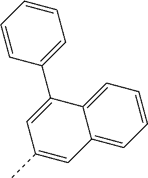 | R2-54 |
| 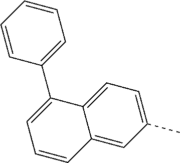 | R2-55 |
| 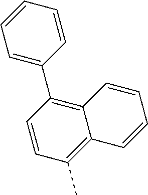 | R2-56 |
| 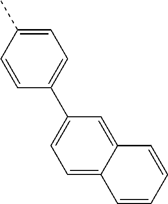 | R2-57 |
| 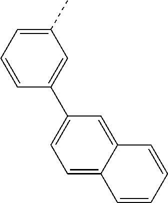 | R2-58 |
| 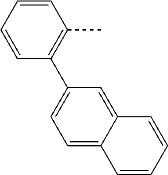 | R2-59 |
| 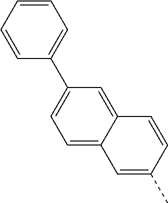 | R2-60 |
| 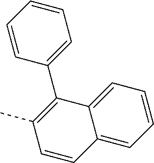 | R2-61 |
| 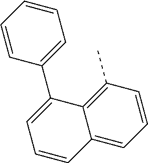 | R2-62 |
| 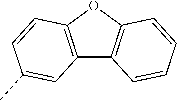 | R2-63 |
| 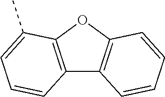 | R2-64 |
| 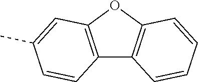 | R2-65 |
| 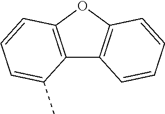 | R2-66 |
| 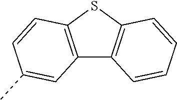 | R2-67 |
| 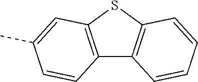 | R2-68 |
| 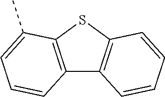 | R2-69 |
| 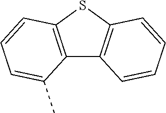 | R2-70 |
| 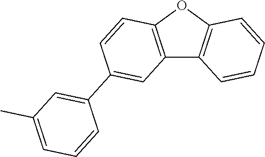 [SMILES: Cc1cccc(-c2ccc3oc4ccccc4c3c2)c1] | R2-71 |
| 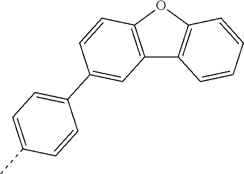 | R2-72 |
| 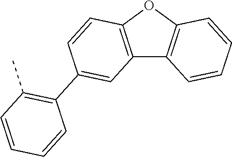 | R2-73 |
| 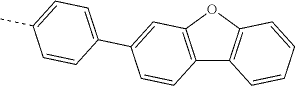 | R2-74 |
| 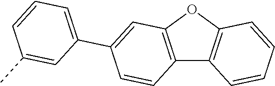 | R2-75 |
| 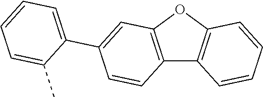 | R2-76 |
| 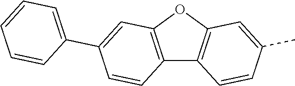 | R2-77 |
| 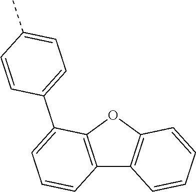 | R2-78 |
| 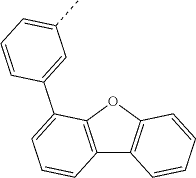 | R2-79 |
| 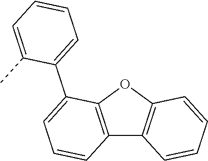 | R2-80 |
| 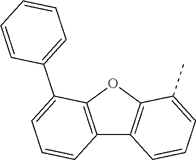 | R2-81 |
| 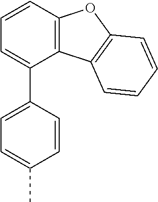 | R2-82 |
| 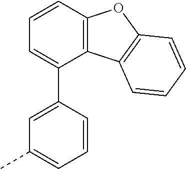 | R2-83 |
| 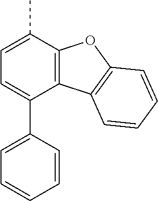 | R2-84 |
| 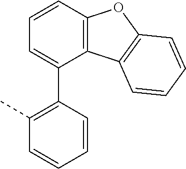 | R2-85 |
| 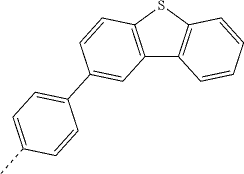 | R2-86 |
| 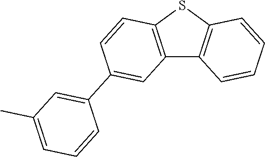 [SMILES: Cc1cccc(-c2ccc3sc4ccccc4c3c2)c1] | R2-87 |
| 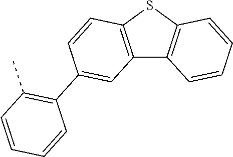 | R2-88 |
| 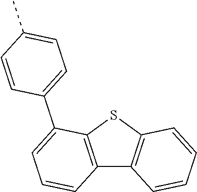 | R2-89 |
| 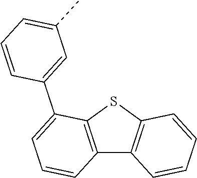 | R2-90 |
| 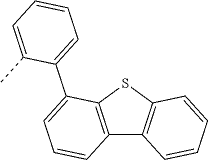 | R2-91 |
| 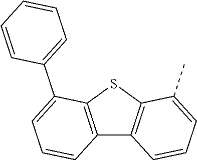 | R2-92 |
| 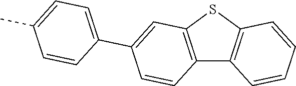 | R2-93 |
| 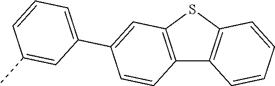 | R2-94 |
| 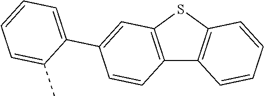 | R2-95 |
| 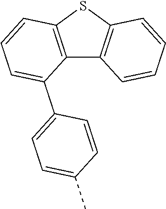 | R2-96 |
| 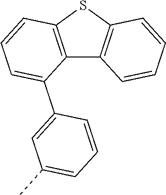 | R2-97 |
| 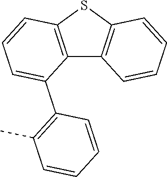 | R2-98 |
| 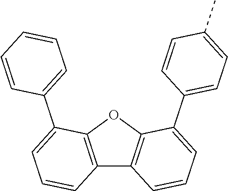 | R2-99 |
| 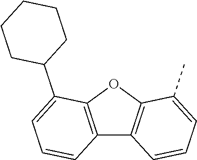 | R2-100 |
| 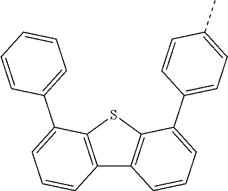 | R2-101 |
| 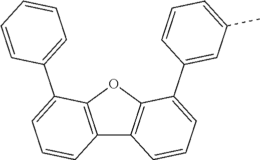 | R2-102 |
| 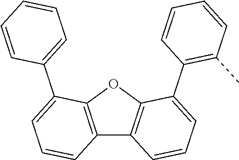 | R2-103 |
| 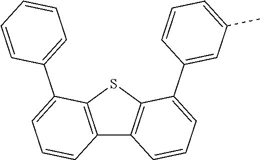 | R2-104 |
| 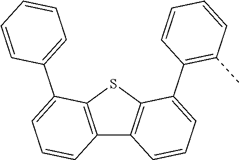 | R2-105 |
| 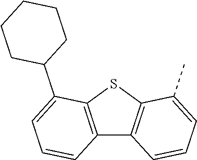 | R2-106 |
| 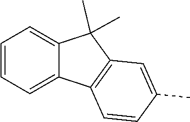 | R2-107 |
| 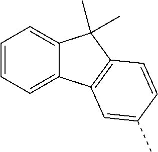 | R2-108 |
| 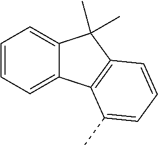 | R2-109 |
| 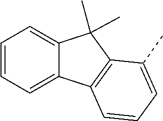 | R2-110 |
| 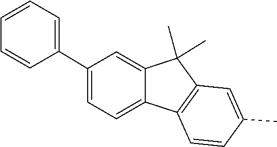 | R2-111 |
| 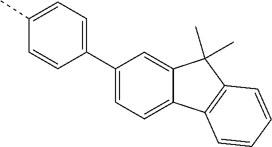 | R2-112 |
| 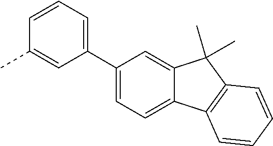 | R2-113 |
| 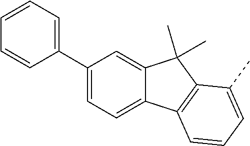 | R2-114 |
| 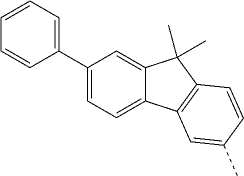 | R2-115 |
| 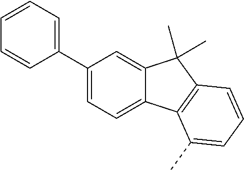 | R2-116 |
| 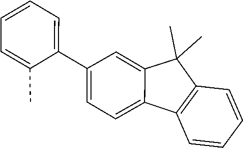 | R2-117 |
| 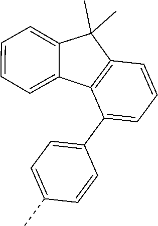 | R2-118 |
| 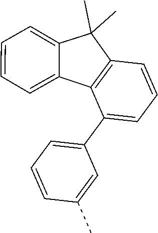 | R2-119 |
| 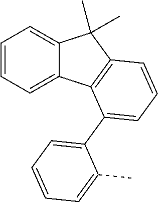 | R2-120 |
| 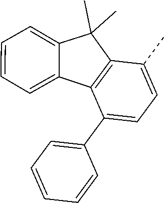 | R2-121 |
| 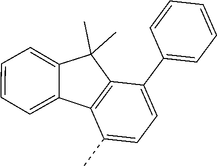 | R2-122 |
| 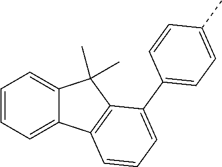 | R2-123 |
| 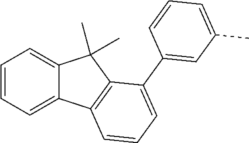 | R2-124 |
| 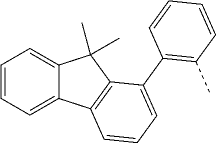 | R2-125 |
| 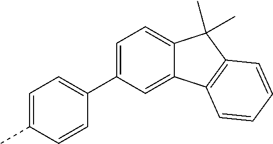 | R2-126 |
| 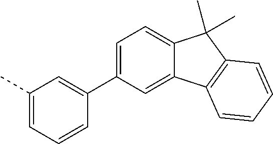 | R2-127 |
| 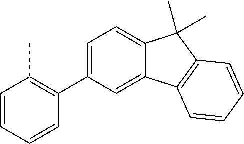 | R2-128 |
| 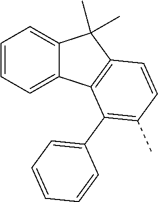 | R2-129 |
| 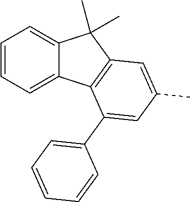 | R2-130 |
| 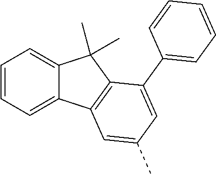 | R2-131 |
| 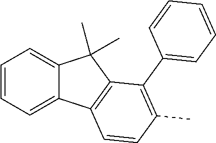 | R2-132 |
| 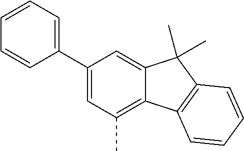 | R2-133 |
| 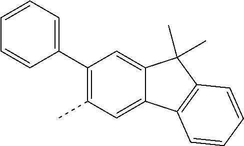 | R2-134 |
| 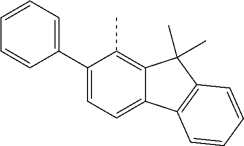 | R2-135 |
| 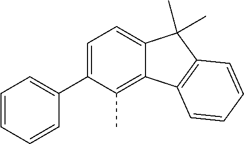 | R2-1336 |
| 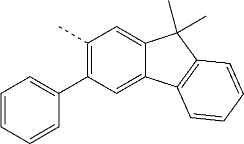 | R2-137 |
| 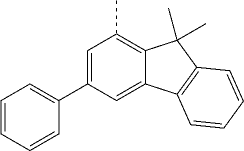 | R2-138 |
| 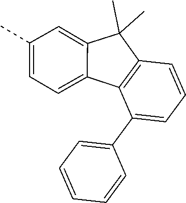 | R2-139 |
| 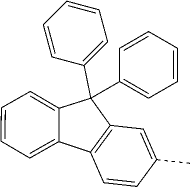 | R2-140 |
| 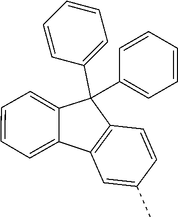 | R2-141 |
| 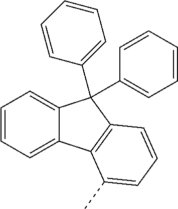 | R2-142 |
| 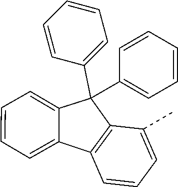 | R2-143 |
| 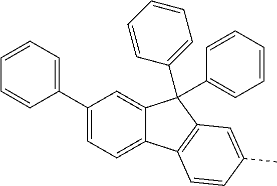 | R2-144 |
| 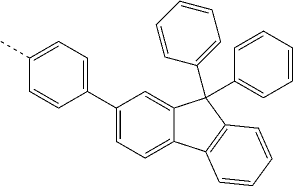 | R2-145 |
| 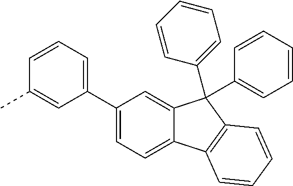 | R2-146 |
| 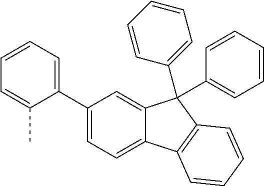 | R2-147 |
| 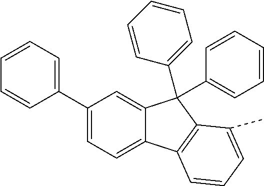 | R2-148 |
| 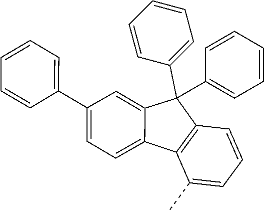 | R2-149 |
| 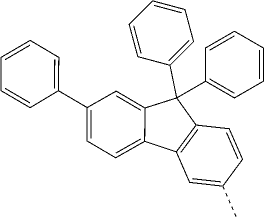 | R2-150 |
| 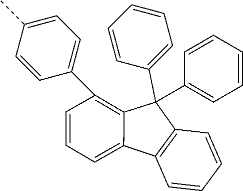 | R2-151 |
| 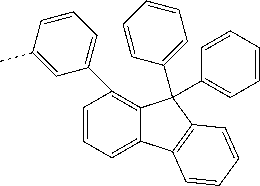 | R2-152 |
| 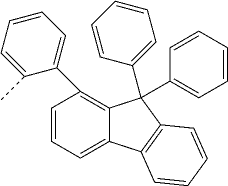 | R2-153 |
| 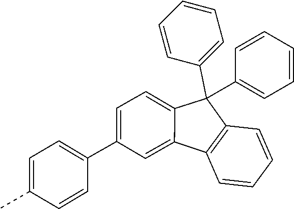 | R2-154 |
| 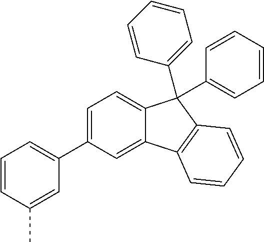 | R2-155 |
| 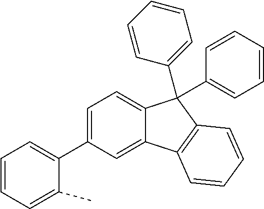 | R2-156 |
| 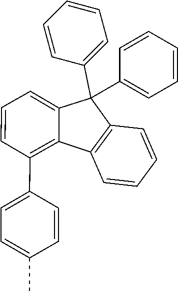 | R2-157 |
| 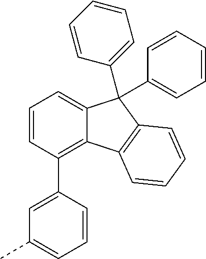 | R2-158 |
| 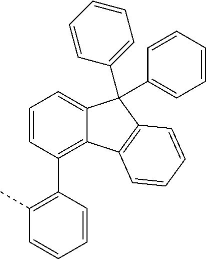 | R2-159 |
| 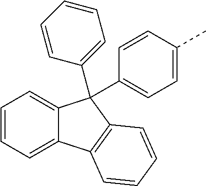 | R2-160 |
| 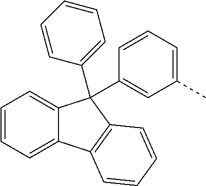 | R2-161 |
| 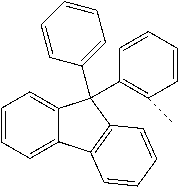 | R2-162 |
| 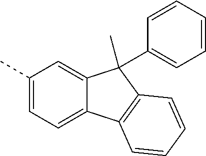 | R2-163 |
| 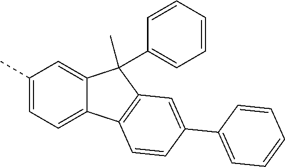 | R2-164 |
| 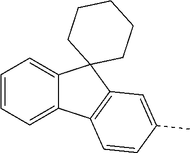 | R2-165 |
| 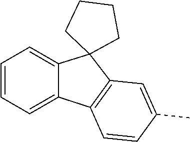 | R2-166 |
| 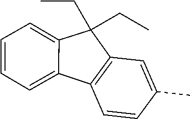 | R2-167 |
| 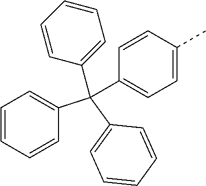 | R2-168 |
| 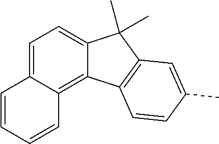 | R2-169 |
| 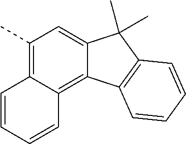 | R2-170 |
| 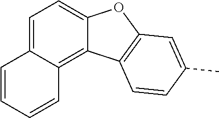 | R2-171 |
| 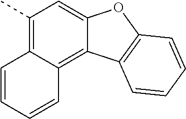 | R2-172 |
| 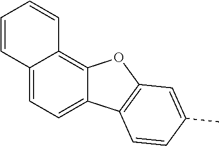 | R2-173 |
| 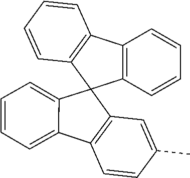 | R2-174 |
| 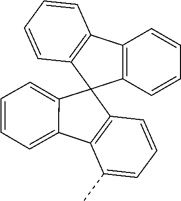 | R2-175 |
| 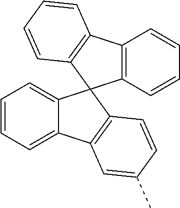 | R2-176 |
| 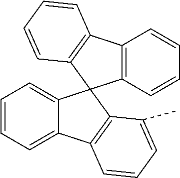 | R2-177 |
| 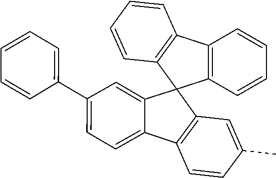 | R2-178 |
| 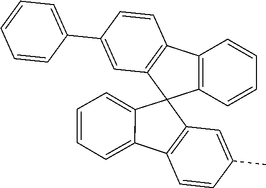 | R2-179 |
| 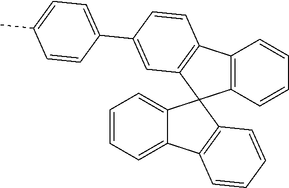 | R2-180 |
| 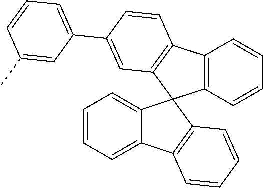 | R2-181 |
| 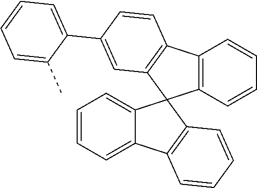 | R2-182 |
| 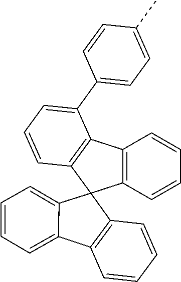 | R2-183 |
| 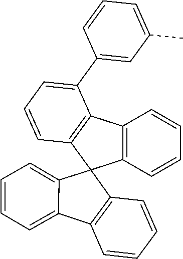 | R2-184 |
| 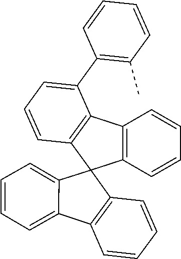 | R2-185 |
| 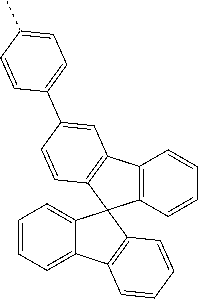 | R2-186 |
| 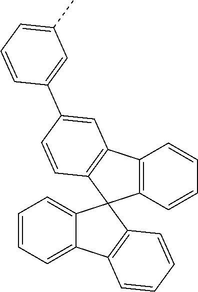 | R2-187 |
| 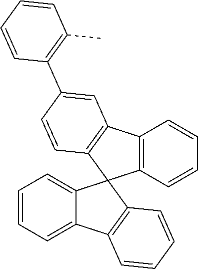 | R2-188 |
| 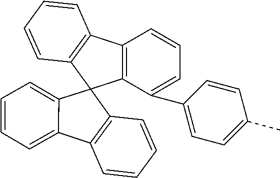 | R2-189 |
| 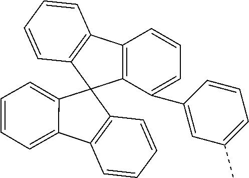 | R2-190 |
| 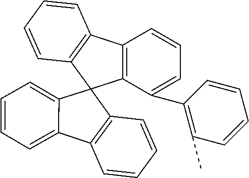 | R2-191 |
| 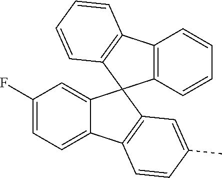 | R2-192 |
| 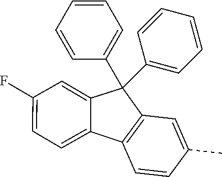 | R2-193 |
| 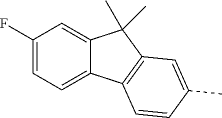 | R2-194 |
| 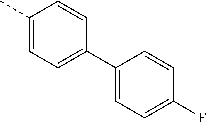 | R2-195 |
| 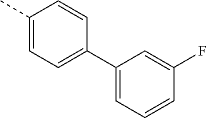 | R2-196 |
| 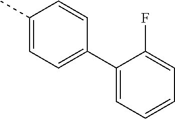 | R2-197 |
| 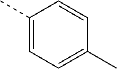 | R2-198 |
| 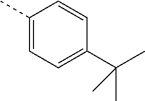 | R2-199 |
| 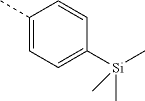 | R2-200 |
| 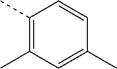 | R2-201 |
| 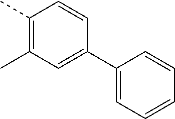 | R2-202 |
| 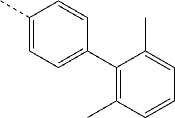 | R2-203 |
| 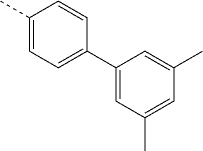 | R2-204 |
| 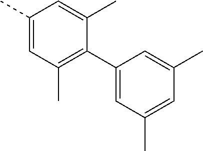 | R2-205 |
| 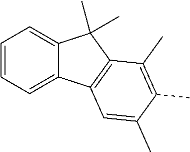 | R2-206 |
| 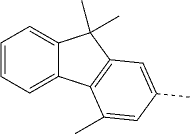 | R2-207 |
| 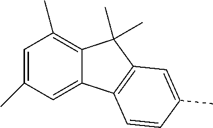 | R2-208 |
| 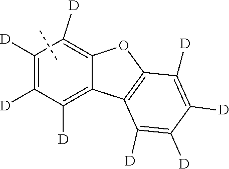 | R2-209 |
| 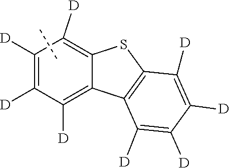 | R2-210 |
| 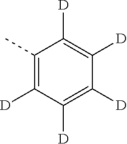 | R2-211 |
| 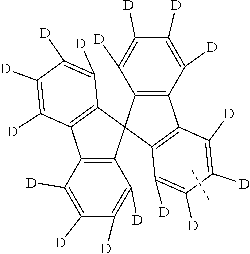 | R2-212 |
| 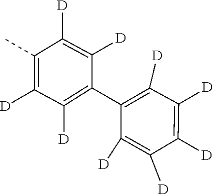 | R2-213 |
| 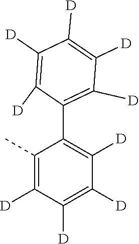 | R2-214 |
| 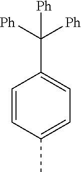 | R2-215 |
| 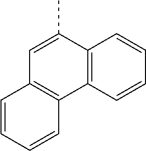 | R2-216 |
| 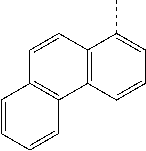 | R2-217 |
| 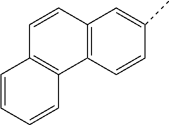 | R2-218 |
| 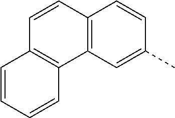 | R2-219 |
| 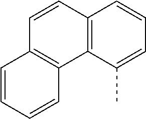 | R2-220 |
| 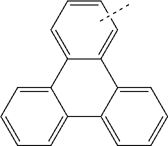 | R2-221 |

where the dashed line represents the bond to the nitrogen atom in formula (5). The substituents R2-1 to R2-221 are preferably partly deuterated or fully deuterated.

In a preferred embodiment of the compounds of the formula (5), Y is O or C(R^{k})₂ and the substituents Rⁱ and R^{j} are H, D or phenyl, more preferably H or D, especially D. The indices q and r are preferably 0 or 1 if R^{h} and/or Rⁱ is a phenyl group and, preferably, q is 3 and r is 4 if R^{h} and/or Rⁱ are D.

Examples of suitable compounds of the formula (2), of the formula (3), of the formula (4) and of the formula (5) that are selected in accordance with the invention are the structures shown below in table 4.

TABLE 4

| | |
|---|---|
| 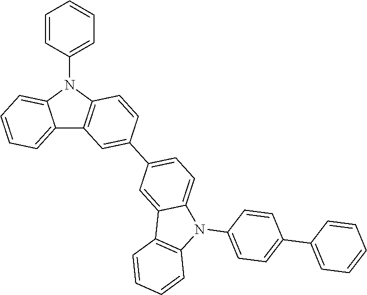 [SMILES: c1ccc(-c2ccc(-n3c4ccccc4c4cc(-c5ccc6c(c5)c5ccccc5n6-c5ccccc5)ccc43)cc2)cc1] | H1 |
| 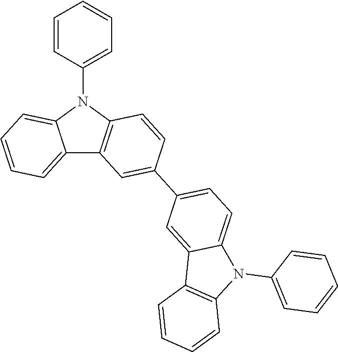 [SMILES: c1ccc(-n2c3ccccc3c3cc(-c4ccc5c(c4)c4ccccc4n5-c4ccccc4)ccc32)cc1] | H2 |
| 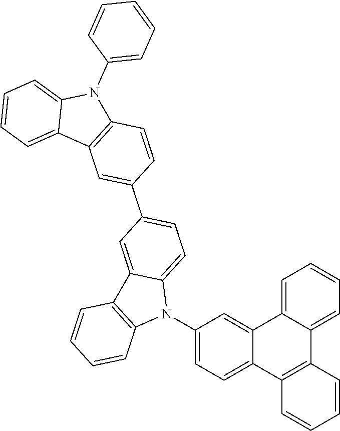 [SMILES: c1ccc(-n2c3ccccc3c3cc(-c4ccc5c(c4)c4ccccc4n5-c4ccc5c6ccccc6c6ccccc6c5c4)ccc32)cc1] | H3 |
| 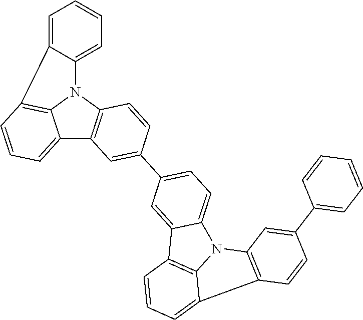 [SMILES: c1ccc(-c2ccc3c4cccc5c6cc(-c7ccc8c(c7)c7cccc9c%10ccccc%10n8c97)ccc6n(c3c2)c54)cc1] | H4 |
| 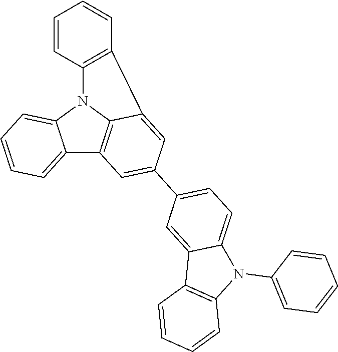 [SMILES: c1ccc(-n2c3ccccc3c3cc(-c4cc5c6ccccc6n6c7ccccc7c(c4)c56)ccc32)cc1] | H5 |
| 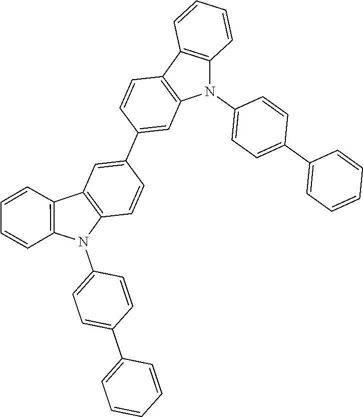 [SMILES: c1ccc(-c2ccc(-n3c4ccccc4c4cc(-c5ccc6c7ccccc7n(-c7ccc(-c8ccccc8)cc7)c6c5)ccc43)cc2)cc1] | H6 |
| 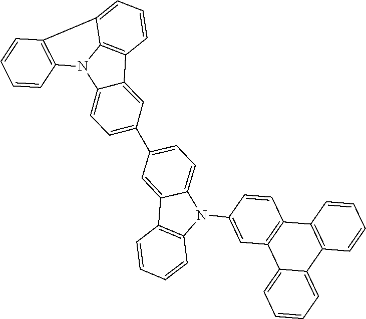 [SMILES: c1ccc2c(c1)c1ccccc1c1cc(-n3c4ccccc4c4cc(-c5ccc6c(c5)c5cccc7c8ccccc8n6c75)ccc43)ccc21] | H7 |
| 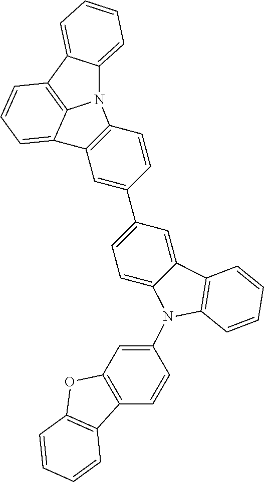 [SMILES: c1ccc2c(c1)oc1cc(-n3c4ccccc4c4cc(-c5ccc6c(c5)c5cccc7c8ccccc8n6c75)ccc43)ccc12] | H8 |
| 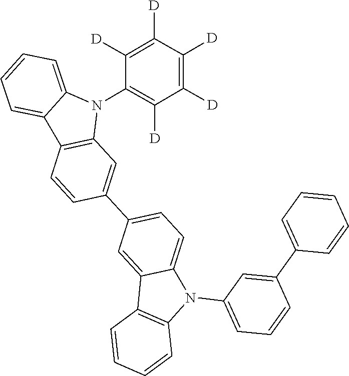 [SMILES: [2H]c1c([2H])c([2H])c(-n2c3ccccc3c3ccc(-c4ccc5c(c4)c4ccccc4n5-c4cccc(-c5ccccc5)c4)cc32)c([2H])c1[2H]] | H9 |
| 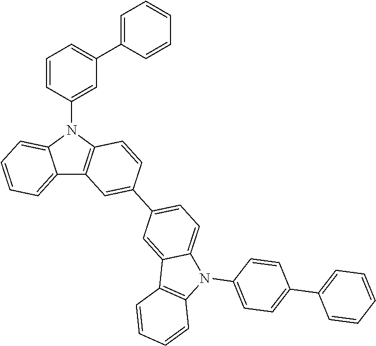 [SMILES: c1ccc(-c2ccc(-n3c4ccccc4c4cc(-c5ccc6c(c5)c5ccccc5n6-c5cccc(-c6ccccc6)c5)ccc43)cc2)cc1] | H10 |
| 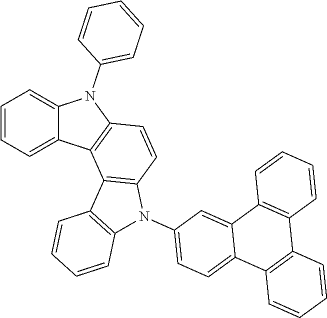 [SMILES: c1ccc(-n2c3ccccc3c3c4c5ccccc5n(-c5ccc6c7ccccc7c7ccccc7c6c5)c4ccc32)cc1] | H11 |
| 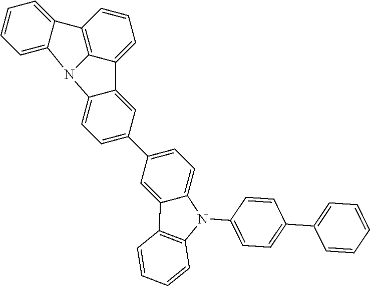 [SMILES: c1ccc(-c2ccc(-n3c4ccccc4c4cc(-c5ccc6c(c5)c5cccc7c8ccccc8n6c75)ccc43)cc2)cc1] | H12 |
| 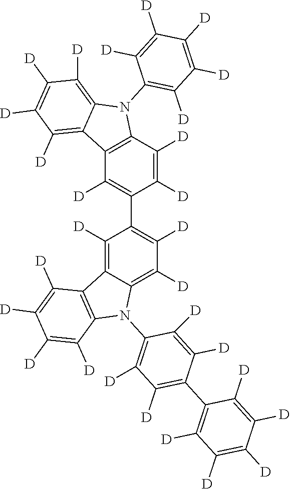 [SMILES: [2H]c1c([2H])c([2H])c(-c2c([2H])c([2H])c(-n3c4c([2H])c([2H])c([2H])c([2H])c4c4c([2H])c(-c5c([2H])c([2H])c6c(c5[2H])c5c([2H])c([2H])c([2H])c([2H])c5n6-c5c([2H])c([2H])c([2H])c([2H])c5[2H])c([2H])c([2H])c43)c([2H])c2[2H])c([2H])c1[2H]] | H13 |
| 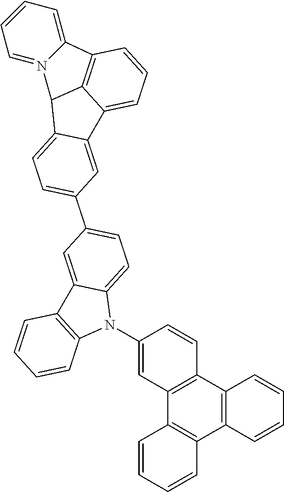 | H14 |
| 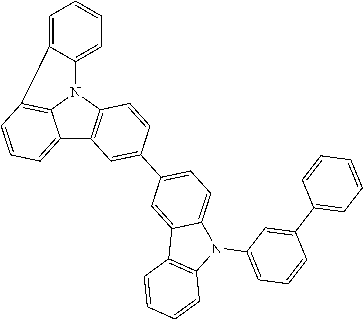 [SMILES: c1ccc(-c2cccc(-n3c4ccccc4c4cc(-c5ccc6c(c5)c5cccc7c8ccccc8n6c75)ccc43)c2)cc1] | H15 |
| 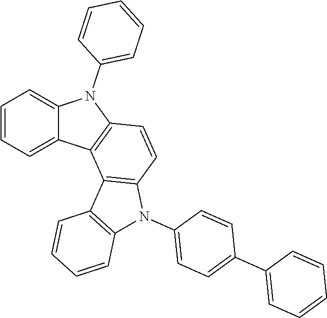 [SMILES: c1ccc(-c2ccc(-n3c4ccccc4c4c5c6ccccc6n(-c6ccccc6)c5ccc43)cc2)cc1] | H16 |
| 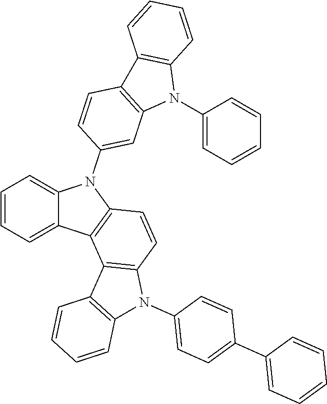 [SMILES: c1ccc(-c2ccc(-n3c4ccccc4c4c5c6ccccc6n(-c6ccc7c8ccccc8n(-c8ccccc8)c7c6)c5ccc43)cc2)cc1] | H17 |
| 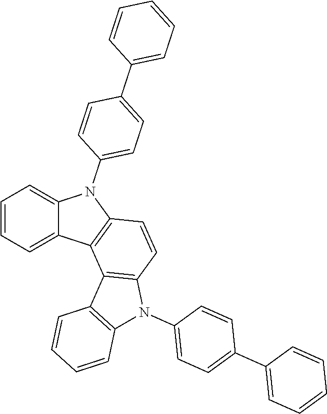 [SMILES: c1ccc(-c2ccc(-n3c4ccccc4c4c5c6ccccc6n(-c6ccc(-c7ccccc7)cc6)c5ccc43)cc2)cc1] | H18 |
| 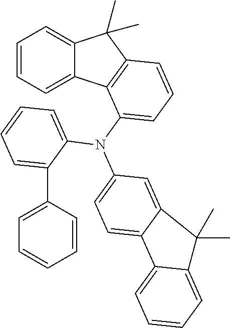 [SMILES: CC1(C)c2ccccc2-c2ccc(N(c3ccccc3-c3ccccc3)c3cccc4c3-c3ccccc3C4(C)C)cc21] | H19 |
| 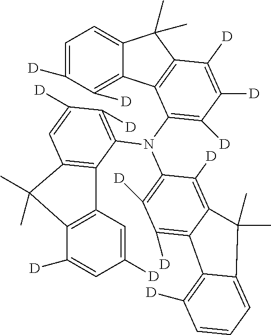 | H20 |
| 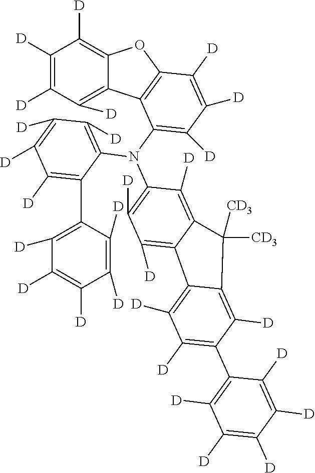 [SMILES: [2H]c1c([2H])c([2H])c(-c2c([2H])c([2H])c3c(c2[2H])C(C([2H])([2H])[2H])(C([2H])([2H])[2H])c2c([2H])c(N(c4c([2H])c([2H])c([2H])c([2H])c4-c4c([2H])c([2H])c([2H])c([2H])c4[2H])c4c([2H])c([2H])c([2H])c5oc6c([2H])c([2H])c([2H])c([2H])c6c45)c([2H])c([2H])c2-3)c([2H])c1[2H]] | H21 |
| 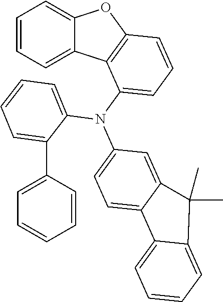 [SMILES: CC1(C)c2ccccc2-c2ccc(N(c3ccccc3-c3ccccc3)c3cccc4oc5ccccc5c34)cc21] | H22 |
| 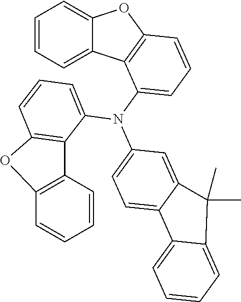 [SMILES: CC1(C)c2ccccc2-c2ccc(N(c3cccc4oc5ccccc5c34)c3cccc4oc5ccccc5c34)cc21] | H23 |
| 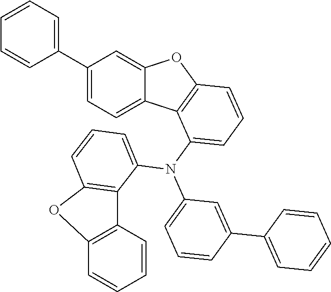 [SMILES: c1ccc(-c2cccc(N(c3cccc4oc5ccccc5c34)c3cccc4oc5cc(-c6ccccc6)ccc5c34)c2)cc1] | H24 |
| 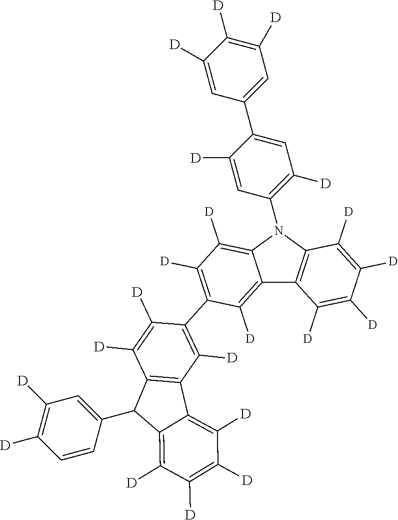 [SMILES: [2H]c1ccc(C2c3c([2H])c([2H])c([2H])c([2H])c3-c3c([2H])c(-c4c([2H])c([2H])c5c(c4[2H])c4c([2H])c([2H])c([2H])c([2H])c4n5-c4cc([2H])c(-c5cc([2H])c([2H])c([2H])c5)cc4[2H])c([2H])c([2H])c32)cc1[2H]] | H25 |
| 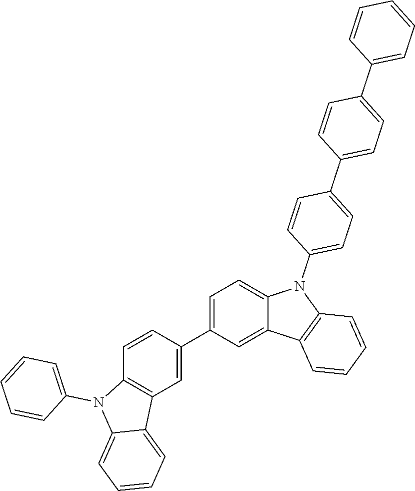 [SMILES: c1ccc(-c2ccc(-c3ccc(-n4c5ccccc5c5cc(-c6ccc7c(c6)c6ccccc6n7-c6ccccc6)ccc54)cc3)cc2)cc1] | H26 |
| 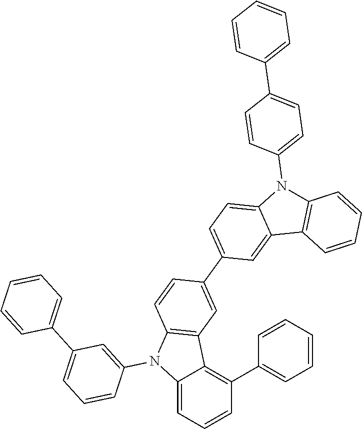 [SMILES: c1ccc(-c2ccc(-n3c4ccccc4c4cc(-c5ccc6c(c5)c5c(-c7ccccc7)cccc5n6-c5cccc(-c6ccccc6)c5)ccc43)cc2)cc1] | H27 |
| 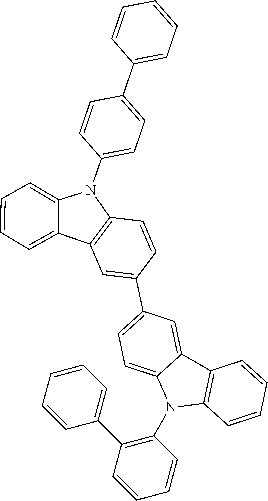 [SMILES: c1ccc(-c2ccc(-n3c4ccccc4c4cc(-c5ccc6c(c5)c5ccccc5n6-c5ccccc5-c5ccccc5)ccc43)cc2)cc1] | H28 |
| 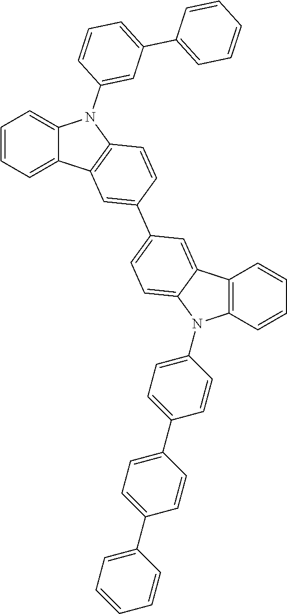 [SMILES: c1ccc(-c2ccc(-c3ccc(-n4c5ccccc5c5cc(-c6ccc7c(c6)c6ccccc6n7-c6cccc(-c7ccccc7)c6)ccc54)cc3)cc2)cc1] | H29 |
| 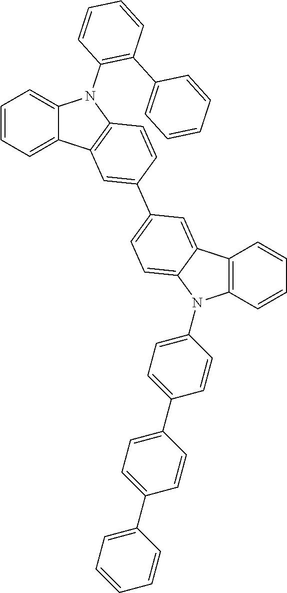 [SMILES: c1ccc(-c2ccc(-c3ccc(-n4c5ccccc5c5cc(-c6ccc7c(c6)c6ccccc6n7-c6ccccc6-c6ccccc6)ccc54)cc3)cc2)cc1] | H30 |
| 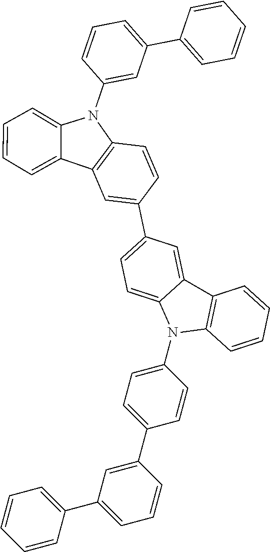 [SMILES: c1ccc(-c2cccc(-c3ccc(-n4c5ccccc5c5cc(-c6ccc7c(c6)c6ccccc6n7-c6cccc(-c7ccccc7)c6)ccc54)cc3)c2)cc1] | H31 |
| 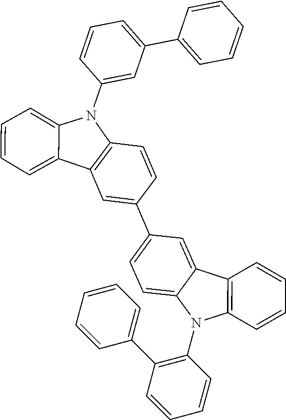 [SMILES: c1ccc(-c2cccc(-n3c4ccccc4c4cc(-c5ccc6c(c5)c5ccccc5n6-c5ccccc5-c5ccccc5)ccc43)c2)cc1] | H32 |
| 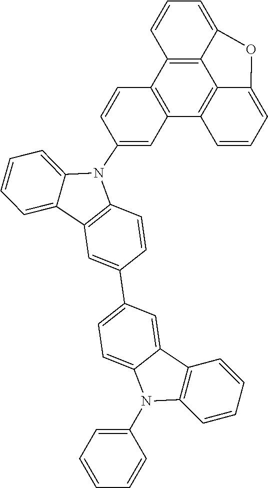 [SMILES: c1ccc(-n2c3ccccc3c3cc(-c4ccc5c(c4)c4ccccc4n5-c4ccc5c(c4)c4cccc6oc7cccc5c7c64)ccc32)cc1] | H33 |

Further particularly preferred compounds of the formula (2) are listed in the following table:

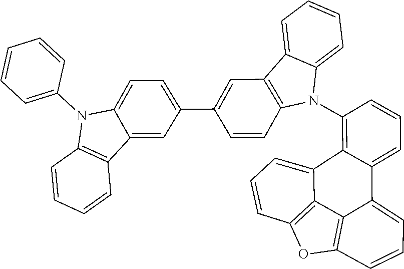 [SMILES: c1ccc(-n2c3ccccc3c3cc(-c4ccc5c(c4)c4ccccc4n5-c4cccc5c6cccc7oc8cccc(c45)c8c76)ccc32)cc1]

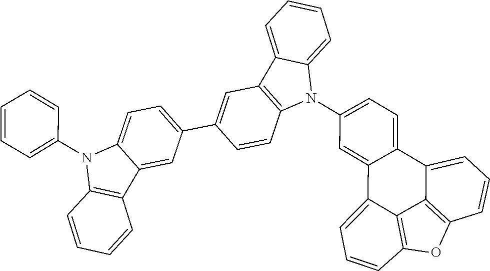 [SMILES: c1ccc(-n2c3ccccc3c3cc(-c4ccc5c(c4)c4ccccc4n5-c4ccc5c(c4)c4cccc6oc7cccc5c7c64)ccc32)cc1]

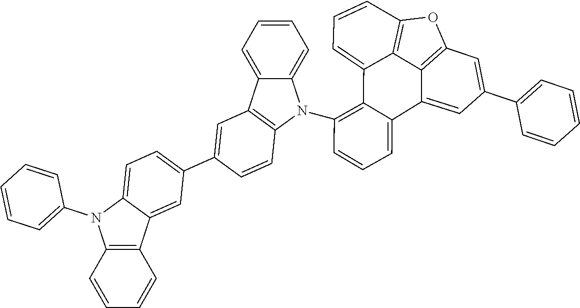 [SMILES: c1ccc(-c2cc3oc4cccc5c6c(-n7c8ccccc8c8cc(-c9ccc%10c(c9)c9ccccc9n%10-c9ccccc9)ccc87)cccc6c(c2)c3c45)cc1]

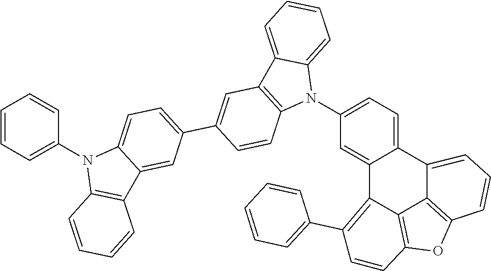 [SMILES: c1ccc(-c2ccc3oc4cccc5c6ccc(-n7c8ccccc8c8cc(-c9ccc%10c(c9)c9ccccc9n%10-c9ccccc9)ccc87)cc6c2c3c45)cc1]

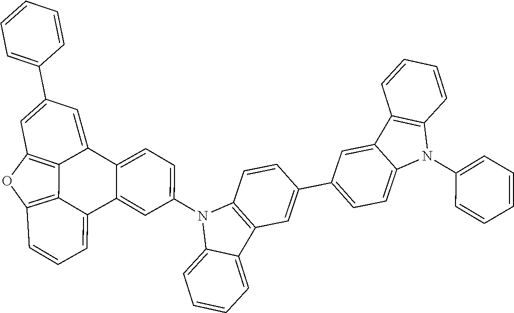 [SMILES: c1ccc(-c2cc3oc4cccc5c6cc(-n7c8ccccc8c8cc(-c9ccc%10c(c9)c9ccccc9n%10-c9ccccc9)ccc87)ccc6c(c2)c3c45)cc1]

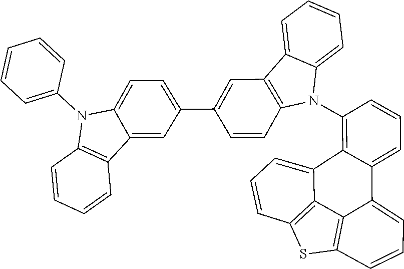 [SMILES: c1ccc(-n2c3ccccc3c3cc(-c4ccc5c(c4)c4ccccc4n5-c4cccc5c6cccc7sc8cccc(c45)c8c76)ccc32)cc1]

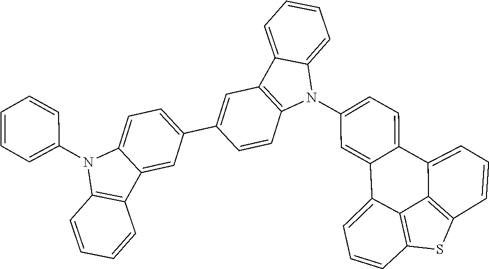 [SMILES: c1ccc(-n2c3ccccc3c3cc(-c4ccc5c(c4)c4ccccc4n5-c4ccc5c(c4)c4cccc6sc7cccc5c7c64)ccc32)cc1]

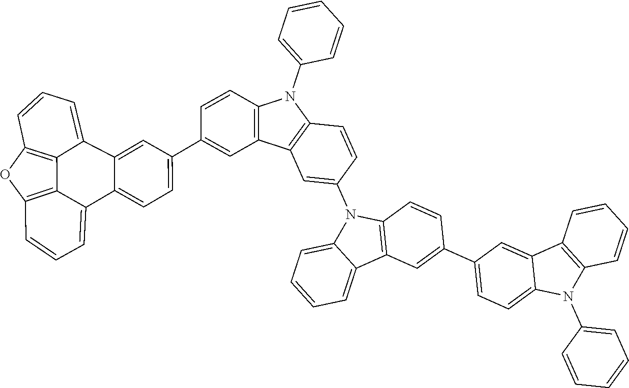 [SMILES: c1ccc(-n2c3ccccc3c3cc(-c4ccc5c(c4)c4ccccc4n5-c4ccc5c(c4)c4cc(-c6ccc7c(c6)c6cccc8oc9cccc7c9c86)ccc4n5-c4ccccc4)ccc32)cc1]

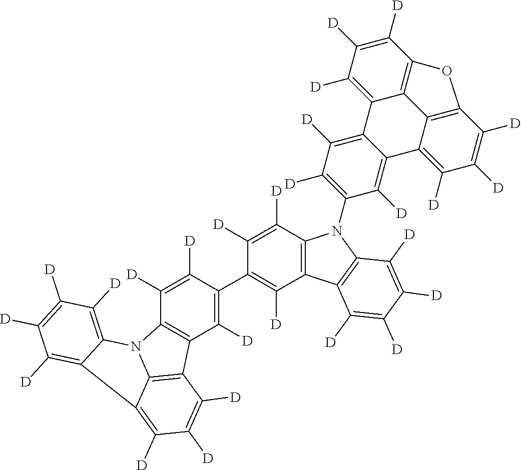 [SMILES: [2H]c1c(-n2c3c([2H])c([2H])c([2H])c([2H])c3c3c([2H])c(-c4c([2H])c([2H])c5c(c4[2H])c4c([2H])c([2H])c([2H])c6c7c([2H])c([2H])c([2H])c([2H])c7n5c64)c([2H])c([2H])c32)c([2H])c2c(c1[2H])c1c([2H])c([2H])c([2H])c3oc4c([2H])c([2H])c([2H])c2c4c31]

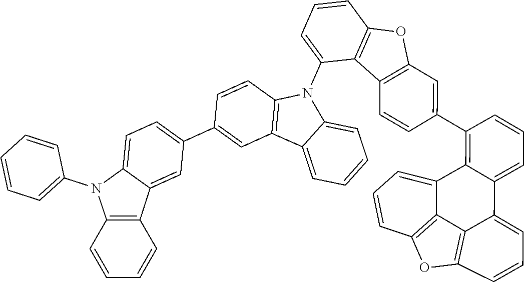 [SMILES: c1ccc(-n2c3ccccc3c3cc(-c4ccc5c(c4)c4ccccc4n5-c4cccc5oc6cc(-c7cccc8c9cccc%10oc%11cccc(c78)c%11c%109)ccc6c45)ccc32)cc1]

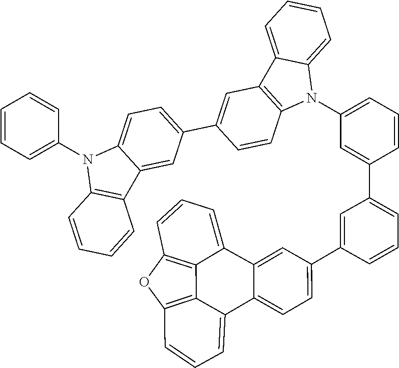 [SMILES: c1ccc(-n2c3ccccc3c3cc(-c4ccc5c(c4)c4ccccc4n5-c4cccc(-c5cccc(-c6ccc7c(c6)c6cccc8oc9cccc7c9c86)c5)c4)ccc32)cc1]

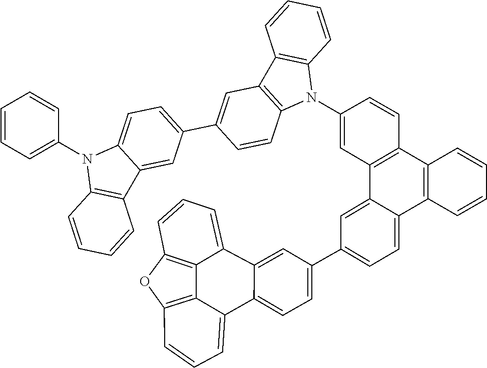 [SMILES: c1ccc(-n2c3ccccc3c3cc(-c4ccc5c(c4)c4ccccc4n5-c4ccc5c6ccccc6c6ccc(-c7ccc8c(c7)c7cccc9oc%10cccc8c%10c97)cc6c5c4)ccc32)cc1]

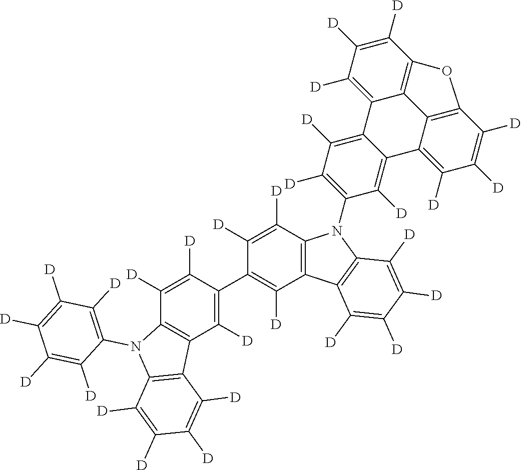 [SMILES: [2H]c1c([2H])c([2H])c(-n2c3c([2H])c([2H])c([2H])c([2H])c3c3c([2H])c(-c4c([2H])c([2H])c5c(c4[2H])c4c([2H])c([2H])c([2H])c([2H])c4n5-c4c([2H])c([2H])c5c(c4[2H])c4c([2H])c([2H])c([2H])c6oc7c([2H])c([2H])c([2H])c5c7c64)c([2H])c([2H])c32)c([2H])c1[2H]]

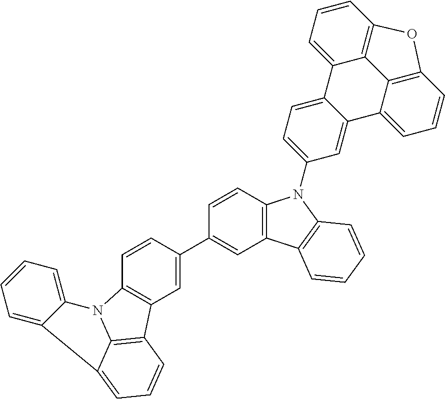 [SMILES: c1cc2oc3cccc4c5cc(-n6c7ccccc7c7cc(-c8ccc9c(c8)c8cccc%10c%11ccccc%11n9c%108)ccc76)ccc5c(c1)c2c34]

The invention further provides an organic electronic device containing an organic layer containing the compositions containing at least one compound of the formula (1) and at least one compound of the formula (2) or of the formula (3) or of the formula (4) or of the formula (5):

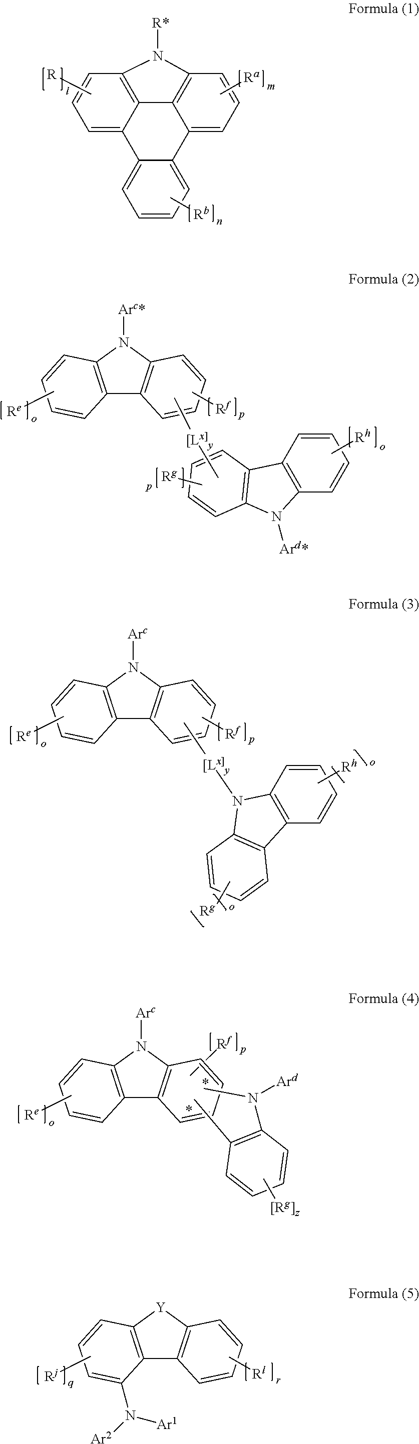

where the symbols and indices used are as follows:
R* is a group of the following formula (1a):

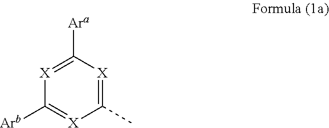

where the dashed bond represents the bond to the nitrogen atom in formula (1);
X is the same or different at each instance and is N or CR^{c}, with the proviso that at least one X group is N and, if X is CR^{c}, this does not form a ring with Ar^{a} or Ar^{b};
Ar^{a}, Ar^{b} are the same or different at each instance and are each independently an aromatic ring system having 6 to 40 aromatic ring atoms or a heteroaromatic ring system having 5 to 40 aromatic ring atoms, each of which may be substituted by one or more R¹ radicals;
L^{x} is the same or different at each instance and is an aromatic ring system having 6 to 40 aromatic ring atoms or a heteroaromatic ring system having 5 to 40 aromatic ring atoms, which may be substituted in each case by one or more R¹ radicals;
Ar^{c}, Ar^{d} are the same or different at each instance and are each independently an aromatic ring system having 6 to 40 aromatic ring atoms or a heteroaromatic ring system having 5 to 40 aromatic ring atoms, each of which may be substituted by one or more R^{d} radicals;
Ar^{c*}, Ar^{d*} are the same or different at each instance and are each independently an aromatic ring system which has 6 to 40 aromatic ring atoms and may be substituted in each case by one or more R^{d} radicals or a heteroaryl group selected from the group consisting of dibenzofuran, dibenzothiophene, carbazole, triphenyleno[1,2-bcd]thiophene, substituted naphtho[1,2,3,4,def]carbazole, phenoxazine, phenothiazine, indolo[3,2,1-jk]carbazole, biscarbazole, benzcarbazole, indenocarbazole, indolocarbazole, benzofurocarbazole, benzothioenocarbazole, dihydroacridine, dihydrophenazine, dibenzodioxin, thianthrene, phenoxathiine, each of which may be substituted by one or more R^{d} radicals, where, independently of one another, an R^{f} radical or an R^{e} radical together with an Ar^{c*} radical or an R^{g} radical or an R^{h} radical together with an Ar^{d*} radical may form a monocyclic or polycyclic, aliphatic, aromatic or heteroaromatic ring, or a group of the formula (6a) or (6b), where the dashed bond represents the bond to the nitrogen atom in the formula (2);

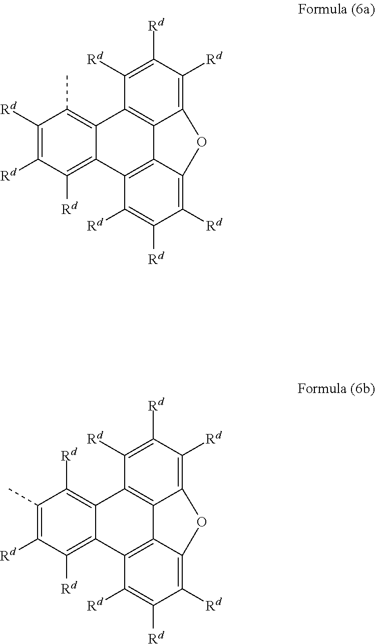

Y is the same or different at each instance and is selected from O, S and C(R^{k})₂;
Ar¹, Ar² are the same or different at each instance and are each independently an aromatic ring system having 6 to 40 aromatic ring atoms or a heteroaromatic ring system having 5 to 40 aromatic ring atoms, each of which may be substituted by one or more R¹ radicals;
R, R^{a}, R^{b} are the same or different at each instance and are each independently H, D, F, Cl, Br, I, N(Ar′)₂, N(R¹)₂, OAr′, SAr′, B(OR¹)₂, CHO, C(═O)R¹, CR¹═C(R¹)₂, CN, C(═O)OR¹, C(═O)NR¹, Si(R¹)₃, NO₂, P(═O)(R¹)₂, OSO₂R¹, OR¹, S(═O)R¹, S(═O)₂R¹, SR¹, a straight-chain alkyl group having 1 to 20 carbon atoms or an alkenyl or alkynyl group having 2 to 20 carbon atoms or a branched or cyclic alkyl group having 3 to 20 carbon atoms, where the alkyl, alkenyl or alkynyl group may in each case be substituted by one or more R¹ radicals, where one or more nonadjacent CH₂ groups may be replaced by —R¹C═CR¹—, —C≡C—, Si(R¹)₂, CONR¹, C═O, C═S, —C(═O)O—, P(═O)(R¹), O, S, SO or SO₂, or an aromatic, heteroaromatic or aromatic ring system having 5 to 40 aromatic ring atoms, each of which may be substituted by one or more R¹ radicals, where two or more R and/or R^{a} and/or R^{b} radicals bonded to the same cycle may together form an aliphatic, heteroaliphatic or aromatic ring system that may be substituted by one or more R¹ radicals, and where two R and/or R^{a} and/or R^{b} radicals bonded to the same carbon, silicon, germanium or tin atom may together form a monocyclic or polycyclic, aliphatic or aromatic ring system that may be substituted by one or more R¹ radicals;
R^{c} is the same or different at each instance and is H, D, F, Cl, Br, I, N(Ar′)₂, N(R¹)₂, OAr′, SAr′, B(OR¹)₂, CHO, C(═O)R¹, CR¹═C(R¹)₂, CN, C(═O)OR¹, C(═O)NR¹, Si(R¹)₃, NO₂, P(═O)(R¹)₂, OSO₂R¹, OR¹, S(═O)R¹, S(═O)₂R¹, SR¹, a straight-chain alkyl group having 1 to 20 carbon atoms or an alkenyl or alkynyl group having 2 to 20 carbon atoms or a branched or cyclic alkyl group having 3 to 20 carbon atoms, where the alkyl, alkenyl or alkynyl group may each case be substituted by one or more R¹ radicals, where one or more nonadjacent CH₂ groups may be replaced by —R¹C═CR¹—, —C≡C—, Si(R¹)₂, NR¹, CONR¹, C═O, C═S, —C(═O)O—, P(═O)(R¹), O, S, SO or SO₂, or an aromatic or heteroaromatic ring system having 5 to 40 aromatic ring atoms, each of which may be substituted by one or more R¹ radicals, where two or more R^{c}, R^{e}, R^{f}, R^{g}, R^{h} or Rⁱ radicals bonded to the same cycle may together form an aliphatic, heteroaliphatic, aromatic or heteroaromatic ring system that may be substituted by one or more R¹ radicals, and where two R^{c}, R^{e}, R^{f}, R^{g}, R^{h} or Rⁱ radicals bonded to the same carbon, silicon, germanium or tin atom may together form a monocyclic or polycyclic, aliphatic, aromatic or heteroaromatic ring system that may be substituted by one or more R¹ radicals;
R^{d} is the same or different at each instance and is H, D, F, Cl, Br, I, N(Ar′)₂, N(R³)₂, OAr′, SAr′, B(OR³)₂, CHO, C(═O)R³, CR³═C(R³)₂, CN, C(═O)OR³, C(═O)NR³, Si(R³)₃, NO₂, P(═O)(R³)₂, OSO₂R³, S(═O)R³, S(═O)₂R³, SR³, a straight-chain alkyl group having 1 to 20 carbon atoms or an alkenyl or alkynyl group having 2 to 20 carbon atoms or a branched or cyclic alkyl group having 3 to 20 carbon atoms, where the alkyl, alkenyl or alkynyl group may be substituted in each case by one or more R¹ radicals, where one or more nonadjacent CH₂ groups may be replaced by —R³C═CR³—, —C≡C—, Si(R³)₂, NR³, CONR³, C═O, C═S, —C(═O)O—, P(═O)(R³), O, S, SO or SO₂, or an aromatic or heteroaromatic ring system which has 5 to 40 aromatic ring atoms and may be substituted in each case by one or more R³ radicals, where two or more R^{d} radicals bonded to the same cycle may together form an aliphatic, heteroaliphatic, aromatic or heteroaromatic ring system which may be substituted by one or more R¹ radicals, and where two R^{d} radicals bonded to the same carbon, silicon, germanium or tin atom may together form a monocyclic or polycyclic, aliphatic, aromatic or heteroaromatic ring system which may be substituted by one or more R³ radicals;
Ar′ is the same or different at each instance and is an aromatic ring system having 6 to 40 aromatic ring atoms or a heteroaromatic ring system having 5 to 40 aromatic ring atoms, which may be substituted by one or more R¹ radicals;
R^{e}, R^{f}, R^{g}, R^{h} are the same or different at each instance and are each independently H, D, F, Cl, Br, I, N(Ar′)₂, N(R¹)₂, OAr′, SAr′, B(OR¹)₂, CHO, C(═O)R¹, CR¹═C(R¹)₂, CN, C(═O)OR¹, C(═O)NR¹, Si(R¹)₃, NO₂, P(═O)(R¹)₂, OSO₂R¹, OR¹, S(═O)R¹, S(═O)₂R¹, SR¹, a straight-chain alkyl group having 1 to 20 carbon atoms or an alkenyl or alkynyl group having 2 to 20 carbon atoms or a branched or cyclic alkyl group having 3 to 20 carbon atoms, where the alkyl, alkenyl or alkynyl group may be substituted in each case by one or more R¹ radicals, where one or more nonadjacent CH₂ groups may be replaced by —R¹C═CR¹—, —C≡C—, Si(R¹)₂, NR¹, CONR¹, C═O, C═S, —C(═O)O—, P(═O)(R¹), O, S, SO or SO₂, or an aromatic or heteroaromatic ring system which has 5 to 40 aromatic ring atoms and may be substituted in each case by one or more R¹ radicals;
Rⁱ, R^{j} are the same or different at each instance and are each independently H, D, F, Cl, Br, I, N(Ar′)₂, N(R¹)₂, OAr′, SAr′, B(OR¹)₂, CHO, C(═O)R¹, CR¹═C(R¹)₂, CN, C(═O)OR¹, C(═O)NR¹, Si(R¹)₃, NO₂, P(═O)(R¹)₂, OSO₂R¹, OR¹, S(═O)R¹, S(═O)₂R¹, SR¹, a straight-chain alkyl group having 1 to 20 carbon atoms or an alkenyl or alkynyl group having 2 to 20 carbon atoms or a branched or cyclic alkyl group having 3 to 20 carbon atoms, where the alkyl, alkenyl or alkynyl group may in each case be substituted by one or more R¹ radicals, where one or more nonadjacent CH₂ groups may be replaced by —R¹C═CR¹—, —C≡C—, Si(R¹)₂, NR¹, CONR¹, C═O, C═S, —C(═O)O—, P(═O)(R¹), O, S, SO or SO₂, or an aromatic or heteroaromatic ring system having 5 to 40 aromatic ring atoms, each of which may be substituted by one or more R¹ radicals, where two or more Rⁱ or R^{j} radicals bonded to the same cycle may together form an aliphatic, heteroaliphatic, aromatic or heteroaromatic ring system that may be substituted by one or more R¹ radicals, and where two Rⁱ or R^{j} radicals bonded to the same carbon, silicon, germanium or tin atom may together form a monocyclic or polycyclic, aliphatic, aromatic or heteroaromatic ring system that may be substituted by one or more R¹ radicals;
R^{k}, R¹ are the same or different at each instance and are D, F, I, B(OR²)₂, N(R²)₂, CHO, C(═O)R², CR²═C(R²)₂, CN, C(═O)OR², Si(R²)₃, NO₂, P(═O)(R²)₂, OSO₂R², SR², OR², S(═O)R², S(═O)₂R², a straight-chain alkyl group having 1 to 20 carbon atoms or an alkenyl or alkynyl group having 2 to 20 carbon atoms or a branched or cyclic alkyl group having 3 to 20 carbon atoms, where the alkyl, alkenyl or alkynyl group may in each case be substituted by one or more R² radicals and where one or more CH₂ groups in the abovementioned groups may be replaced by —R²C═CR²—, —C≡C—, Si(R²)₂, C═O, C═S, —C(═O)O—, NR², CONR², P(═O)(R²), O, S, SO or SO₂, and where one or more hydrogen atoms in the abovementioned groups may be replaced by D, F, Cl, Br, I, CN or NO₂, or an aromatic or heteroaromatic ring system which has 5 to 30 aromatic ring atoms and may be substituted in each case by one or more R² radicals, where two or more R^{k} or R¹ radicals together may form an aliphatic, heteroaliphatic, aromatic or heteroaromatic ring system;
R² is the same or different at each instance and is D, F, CN or an aliphatic, aromatic or heteroaromatic organic radical having 1 to 20 carbon atoms, in which one or more hydrogen atoms may also be replaced by D or F; at the same time, two or more R² substituents may be joined to one another and may form a ring;
R³ is the same or different at each instance and is D, CN or an aliphatic, aromatic or heteroaromatic organic radical having 1 to 20 carbon atoms; at the same time, two or more R³ substituents may be joined to one another and may form a ring;
l, m, p, q are the same or different at each instance and are independently 0, 1, 2 or 3;
n, o, r, z are the same or different at each instance and are independently 0, 1, 2, 3 or 4;
y at each instance is independently 0 or 1.

In a preferred embodiment of the organic electronic device containing an organic layer containing the composition containing at least a compound of the formula (1) and a compound of the formula (2) or of the formula (3) or of the formula (4) or of the formula (5), the composition is preferably present in the emission layer, especially as host material in the emission layer together with a phosphorescent emitter.

The remarks with regard to the materials of the formulae (1), (2), (3), (4) and (5) and preferred embodiments thereof are correspondingly applicable to the composition, and to the organic electronic device containing this composition.

Particularly preferred compositions of the materials of the formula (1) with the materials of the formula (2) or of the formula (3) or of the formula (4) or of the formula (5) for the device of the invention are obtained by combination of the compounds E1 to E18 with H1 to H33, as shown below in table 5.

TABLE 5

| | | | | | | | | |
|---|---|---|---|---|---|---|---|---|
| M1 | E1 | H1 | M2 | E2 | H1 | M3 | E3 | H1 |
| M4 | E4 | H1 | M5 | E5 | H1 | M6 | E6 | H1 |
| M7 | E7 | H1 | M8 | E8 | H1 | M9 | E9 | H1 |
| M10 | E10 | H2 | M11 | E11 | H1 | M12 | E12 | H1 |
| M13 | E13 | H3 | M14 | E14 | H1 | M15 | E15 | H1 |
| M16 | E16 | H4 | M17 | E17 | H1 | M18 | E18 | H1 |
| M19 | E1 | H2 | M20 | E2 | H2 | M21 | E3 | H2 |
| M22 | E4 | H2 | M23 | E5 | H2 | M24 | E6 | H2 |
| M25 | E7 | H2 | M26 | E8 | H2 | M27 | E9 | H2 |
| M28 | E10 | H2 | M29 | E11 | H2 | M30 | E12 | H2 |
| M31 | E13 | H2 | M32 | E14 | H2 | M33 | E15 | H2 |
| M34 | E16 | H2 | M35 | E17 | H2 | M36 | E18 | H2 |
| M37 | E1 | H3 | M38 | E2 | H3 | M39 | E3 | H3 |
| M40 | E4 | H3 | M41 | E5 | H3 | M42 | E6 | H3 |
| M43 | E7 | H3 | M44 | E8 | H3 | M45 | E9 | H3 |
| M46 | E10 | H3 | M47 | E11 | H3 | M48 | E12 | H3 |
| M49 | E13 | H3 | M50 | E14 | H3 | M51 | E15 | H3 |
| M52 | E16 | H3 | M53 | E17 | H3 | M54 | E18 | H3 |
| M55 | E1 | H4 | M56 | E2 | H4 | M57 | E3 | H4 |
| M58 | E4 | H4 | M59 | E5 | H4 | M60 | E6 | H4 |
| M61 | E7 | H4 | M62 | E8 | H4 | M63 | E9 | H4 |
| M64 | E10 | H4 | M65 | E11 | H4 | M66 | E12 | H4 |
| M67 | E13 | H4 | M68 | E14 | H4 | M69 | E15 | H4 |
| M70 | E16 | H4 | M71 | E17 | H4 | M72 | E18 | H4 |
| M73 | E1 | H5 | M74 | E2 | H5 | M75 | E3 | H5 |
| M76 | E4 | H5 | M77 | E5 | H5 | M78 | E6 | H5 |
| M79 | E7 | H5 | M80 | E8 | H5 | M81 | E9 | H5 |
| M82 | E10 | H5 | M83 | E11 | H5 | M84 | E12 | H5 |
| M85 | E13 | H5 | M86 | E14 | H5 | M87 | E15 | H5 |
| M88 | E16 | H5 | M89 | E17 | H5 | M90 | E18 | H5 |
| M91 | E1 | H6 | M92 | E2 | H6 | M93 | E3 | H6 |
| M94 | E4 | H6 | M95 | E5 | H6 | M96 | E6 | H6 |
| M97 | E7 | H6 | M98 | E8 | H6 | M99 | E9 | H6 |
| M100 | E10 | H6 | M101 | E11 | H6 | M102 | E12 | H6 |
| M103 | E13 | H6 | M104 | E14 | H6 | M105 | E15 | H6 |
| M106 | E16 | H6 | M107 | E17 | H6 | M108 | E18 | H6 |
| M109 | E1 | H7 | M110 | E2 | H7 | M111 | E3 | H7 |
| M112 | E4 | H7 | M113 | E5 | H7 | M114 | E6 | H7 |
| M115 | E7 | H7 | M116 | E8 | H7 | M117 | E9 | H7 |
| M118 | E10 | H7 | M119 | E11 | H7 | M120 | E12 | H7 |
| M121 | E13 | H7 | M122 | E14 | H7 | M123 | E15 | H7 |
| M124 | E16 | H7 | M125 | E17 | H7 | M126 | E18 | H7 |
| M127 | E1 | H8 | M128 | E2 | H8 | M129 | E3 | H8 |
| M130 | E4 | H8 | M131 | E5 | H8 | M132 | E6 | H8 |
| M133 | E7 | H8 | M134 | E8 | H8 | M135 | E9 | H8 |
| M136 | E10 | H8 | M137 | E11 | H8 | M138 | E12 | H8 |
| M139 | E13 | H8 | M140 | E14 | H8 | M141 | E15 | H8 |
| M142 | E16 | H8 | M143 | E17 | H8 | M144 | E18 | H8 |
| M145 | E1 | H9 | M146 | E2 | H9 | M147 | E3 | H9 |
| M148 | E4 | H9 | M149 | E5 | H9 | M150 | E6 | H9 |
| M151 | E7 | H9 | M152 | E8 | H9 | M153 | E9 | H9 |
| M154 | E10 | H9 | M155 | E11 | H9 | M156 | E12 | H9 |
| M157 | E13 | H9 | M158 | E14 | H9 | M159 | E15 | H9 |
| M160 | E16 | H9 | M161 | E17 | H9 | M162 | E18 | H9 |
| M163 | E1 | H10 | M164 | E2 | H10 | M165 | E3 | H10 |
| M166 | E4 | H10 | M167 | E5 | H10 | M168 | E6 | H10 |
| M169 | E7 | H10 | M170 | E8 | H10 | M171 | E9 | H10 |
| M172 | E10 | H10 | M173 | E11 | H10 | M174 | E12 | H10 |
| M175 | E13 | H10 | M176 | E14 | H10 | M177 | E15 | H10 |
| M178 | E16 | H10 | M179 | E17 | H10 | M180 | E18 | H10 |
| M181 | E1 | H11 | M182 | E2 | H11 | M183 | E3 | H11 |
| M184 | E4 | H11 | M185 | E5 | H11 | M186 | E6 | H11 |
| M187 | E7 | H11 | M188 | E8 | H11 | M189 | E9 | H11 |
| M190 | E10 | H11 | M191 | E11 | H11 | M192 | E12 | H11 |
| M193 | E13 | H11 | M194 | E14 | H11 | M195 | E15 | H11 |
| M196 | E16 | H11 | M197 | E17 | H11 | M198 | E18 | H11 |
| M199 | E1 | H12 | M200 | E2 | H12 | M201 | E3 | H12 |
| M202 | E4 | H12 | M203 | E5 | H12 | M204 | E6 | H12 |
| M205 | E7 | H12 | M206 | E8 | H12 | M207 | E9 | H12 |
| M208 | E10 | H12 | M209 | E11 | H12 | M210 | E12 | H12 |
| M211 | E13 | H12 | M212 | E14 | H12 | M213 | E15 | H12 |
| M214 | E16 | H12 | M215 | E17 | H12 | M216 | E18 | H12 |
| M217 | E1 | H13 | M218 | E2 | H13 | M219 | E3 | H13 |
| M220 | E4 | H13 | M221 | E5 | H13 | M222 | E6 | H13 |
| M223 | E7 | H13 | M224 | E8 | H13 | M225 | E9 | H13 |
| M226 | E10 | H13 | M227 | E11 | H13 | M228 | E12 | H13 |
| M229 | E13 | H13 | M230 | E14 | H13 | M231 | E15 | H13 |
| M232 | E16 | H13 | M233 | E17 | H13 | M234 | E18 | H13 |
| M235 | E1 | H14 | M236 | E2 | H14 | M237 | E3 | H14 |
| M238 | E4 | H14 | M239 | E5 | H14 | M240 | E6 | H14 |
| M241 | E7 | H14 | M242 | E8 | H14 | M243 | E9 | H14 |
| M244 | E10 | H14 | M245 | E11 | H14 | M246 | E12 | H14 |
| M247 | E13 | H14 | M248 | E14 | H14 | M249 | E15 | H14 |
| M250 | E16 | H14 | M251 | E17 | H14 | M252 | E18 | H14 |
| M253 | E1 | H15 | M254 | E2 | H15 | M255 | E3 | H15 |
| M256 | E4 | H15 | M257 | E5 | H15 | M258 | E6 | H15 |
| M259 | E7 | H15 | M260 | E8 | H15 | M261 | E9 | H15 |
| M262 | E10 | H15 | M263 | E11 | H15 | M264 | E12 | H15 |
| M265 | E13 | H15 | M266 | E14 | H15 | M267 | E15 | H15 |
| M268 | E16 | H15 | M269 | E17 | H15 | M270 | E18 | H15 |
| M271 | E1 | H16 | M272 | E2 | H16 | M273 | E3 | H16 |
| M274 | E4 | H16 | M275 | E5 | H16 | M276 | E6 | H16 |
| M277 | E7 | H16 | M278 | E8 | H16 | M279 | E9 | H16 |
| M280 | E10 | H16 | M281 | E11 | H16 | M282 | E12 | H16 |
| M283 | E13 | H16 | M284 | E14 | H16 | M285 | E15 | H16 |
| M286 | E16 | H16 | M287 | E17 | H16 | M288 | E18 | H16 |
| M289 | E1 | H17 | M290 | E2 | H17 | M291 | E3 | H17 |
| M292 | E4 | H17 | M293 | E5 | H17 | M294 | E6 | H17 |
| M295 | E7 | H17 | M296 | E8 | H17 | M297 | E9 | H17 |
| M298 | E10 | H17 | M299 | E11 | H17 | M300 | E12 | H17 |
| M301 | E13 | H17 | M302 | E14 | H17 | M303 | E15 | H17 |
| M304 | E16 | H17 | M305 | E17 | H17 | M306 | E18 | H17 |
| M307 | E1 | H18 | M308 | E2 | H18 | M309 | E3 | H18 |
| M310 | E4 | H18 | M311 | E5 | H18 | M312 | E6 | H18 |
| M313 | E7 | H18 | M314 | E8 | H18 | M315 | E9 | H18 |
| M316 | E10 | H18 | M317 | E11 | H18 | M318 | E12 | H18 |
| M319 | E13 | H18 | M320 | E14 | H18 | M321 | E15 | H18 |
| M322 | E16 | H18 | M323 | E17 | H18 | M324 | E18 | H18 |
| M325 | E1 | H19 | M326 | E2 | H19 | M327 | E3 | H19 |
| M328 | E4 | H19 | M329 | E5 | H19 | M330 | E6 | H19 |
| M331 | E7 | H19 | M332 | E8 | H19 | M333 | E9 | H19 |
| M334 | E10 | H19 | M335 | E11 | H19 | M336 | E12 | H19 |
| M337 | E13 | H19 | M338 | E14 | H19 | M339 | E15 | H19 |
| M340 | E16 | H19 | M341 | E17 | H19 | M342 | E18 | H19 |
| M343 | E1 | H20 | M344 | E2 | H20 | M345 | E3 | H20 |
| M346 | E4 | H20 | M347 | E5 | H20 | M348 | E6 | H20 |
| M349 | E7 | H20 | M350 | E8 | H20 | M351 | E9 | H20 |
| M352 | E10 | H20 | M353 | E11 | H20 | M354 | E12 | H20 |
| M355 | E13 | H20 | M356 | E14 | H20 | M357 | E15 | H20 |
| M358 | E16 | H20 | M359 | E17 | H20 | M360 | E18 | H20 |
| M361 | E1 | H21 | M362 | E2 | H21 | M363 | E3 | H21 |
| M364 | E4 | H21 | M365 | E5 | H21 | M366 | E6 | H21 |
| M367 | E7 | H21 | M368 | E8 | H21 | M369 | E9 | H21 |
| M370 | E10 | H21 | M371 | E11 | H21 | M372 | E12 | H21 |
| M373 | E13 | H21 | M374 | E14 | H21 | M375 | E15 | H21 |
| M376 | E16 | H21 | M377 | E17 | H21 | M378 | E18 | H21 |
| M379 | E1 | H22 | M380 | E2 | H22 | M381 | E3 | H22 |
| M382 | E4 | H22 | M383 | E5 | H22 | M384 | E6 | H22 |
| M385 | E7 | H22 | M386 | E8 | H22 | M387 | E9 | H22 |
| M388 | E10 | H22 | M389 | E11 | H22 | M390 | E12 | H22 |
| M391 | E13 | H22 | M392 | E14 | H22 | M393 | E15 | H22 |
| M394 | E16 | H22 | M395 | E17 | H22 | M396 | E18 | H22 |
| M397 | E1 | H23 | M398 | E2 | H23 | M399 | E3 | H23 |
| M400 | E4 | H23 | M401 | E5 | H23 | M402 | E6 | H23 |
| M403 | E7 | H23 | M404 | E8 | H23 | M405 | E9 | H23 |
| M406 | E10 | H23 | M407 | E11 | H23 | M408 | E12 | H23 |
| M409 | E13 | H23 | M410 | E14 | H23 | M411 | E15 | H23 |
| M412 | E16 | H23 | M413 | E17 | H23 | M414 | E18 | H23 |
| M415 | E1 | H24 | M416 | E2 | H24 | M417 | E3 | H24 |
| M418 | E4 | H24 | M419 | E5 | H24 | M420 | E6 | H24 |
| M421 | E7 | H24 | M422 | E8 | H24 | M423 | E9 | H24 |
| M424 | E10 | H24 | M425 | E11 | H24 | M426 | E12 | H24 |
| M427 | E13 | H24 | M428 | E14 | H24 | M429 | E15 | H24 |
| M430 | E16 | H24 | M431 | E17 | H24 | M432 | E18 | H24 |
| M433 | E1 | H25 | M434 | E2 | H25 | M435 | E3 | H25 |
| M436 | E4 | H25 | M437 | E5 | H25 | M438 | E6 | H25 |
| M439 | E7 | H25 | M440 | E8 | H25 | M441 | E9 | H25 |
| M442 | E10 | H25 | M443 | E11 | H25 | M444 | E12 | H25 |
| M445 | E13 | H25 | M446 | E14 | H25 | M447 | E15 | H25 |
| M448 | E16 | H25 | M449 | E17 | H25 | M450 | E18 | H25 |
| M451 | E1 | H26 | M452 | E2 | H26 | M453 | E3 | H26 |
| M454 | E4 | H26 | M455 | E5 | H26 | M456 | E6 | H26 |
| M457 | E7 | H26 | M458 | E8 | H26 | M459 | E9 | H26 |
| M460 | E10 | H26 | M461 | E11 | H26 | M462 | E12 | H26 |
| M463 | E13 | H26 | M464 | E14 | H26 | M465 | E15 | H26 |
| M466 | E16 | H26 | M467 | E17 | H26 | M468 | E18 | H26 |
| M469 | E1 | H27 | M470 | E2 | H27 | M471 | E3 | H27 |
| M472 | E4 | H27 | M473 | E5 | H27 | M474 | E6 | H27 |
| M475 | E7 | H27 | M476 | E8 | H27 | M477 | E9 | H27 |
| M478 | E10 | H27 | M479 | E11 | H27 | M480 | E12 | H27 |
| M481 | E13 | H27 | M482 | E14 | H27 | M483 | E15 | H27 |
| M484 | E16 | H27 | M485 | E17 | H27 | M486 | E18 | H27 |
| M487 | E1 | H28 | M488 | E2 | H28 | M489 | E3 | H28 |
| M490 | E4 | H28 | M491 | E5 | H28 | M492 | E6 | H28 |
| M493 | E7 | H28 | M494 | E8 | H28 | M495 | E9 | H28 |
| M496 | E10 | H28 | M497 | E11 | H28 | M498 | E12 | H28 |
| M499 | E13 | H28 | M500 | E14 | H28 | M501 | E15 | H28 |
| M502 | E16 | H28 | M503 | E17 | H28 | M504 | E18 | H28 |
| M505 | E1 | H29 | M506 | E2 | H29 | M507 | E3 | H29 |
| M508 | E4 | H29 | M509 | E5 | H29 | M510 | E6 | H29 |
| M511 | E7 | H29 | M512 | E8 | H29 | M513 | E9 | H29 |
| M514 | E10 | H29 | M515 | E11 | H29 | M516 | E12 | H29 |
| M517 | E13 | H29 | M518 | E14 | H29 | M519 | E15 | H29 |
| M520 | E16 | H29 | M521 | E17 | H29 | M522 | E18 | H29 |
| M523 | E1 | H30 | M524 | E2 | H30 | M525 | E3 | H30 |
| M526 | E4 | H30 | M527 | E5 | H30 | M528 | E6 | H30 |
| M529 | E7 | H30 | M530 | E8 | H30 | M531 | E9 | H30 |
| M532 | E10 | H30 | M533 | E11 | H30 | M534 | E12 | H30 |
| M535 | E13 | H30 | M536 | E14 | H30 | M537 | E15 | H30 |
| M538 | E16 | H30 | M539 | E17 | H30 | M540 | E18 | H30 |
| M541 | E1 | H31 | M542 | E2 | H31 | M543 | E3 | H31 |
| M544 | E4 | H31 | M545 | E5 | H31 | M546 | E6 | H31 |
| M547 | E7 | H31 | M548 | E8 | H31 | M549 | E9 | H31 |
| M550 | E10 | H31 | M551 | E11 | H31 | M552 | E12 | H31 |
| M553 | E13 | H31 | M554 | E14 | H31 | M555 | E15 | H31 |
| M556 | E16 | H31 | M557 | E17 | H31 | M558 | E18 | H31 |
| M559 | E1 | H32 | M560 | E2 | H32 | M561 | E3 | H32 |
| M562 | E4 | H32 | M563 | E5 | H32 | M564 | E6 | H32 |
| M565 | E7 | H32 | M566 | E8 | H32 | M567 | E9 | H32 |
| M568 | E10 | H32 | M569 | E11 | H32 | M570 | E12 | H32 |
| M571 | E13 | H32 | M572 | E14 | H32 | M573 | E15 | H32 |
| M574 | E16 | H32 | M575 | E17 | H32 | M576 | E18 | H32 |
| M577 | E1 | H33 | M578 | E2 | H33 | M579 | E3 | H33 |
| M580 | E4 | H33 | M581 | E5 | H33 | M582 | E6 | H33 |
| M583 | E7 | H33 | M584 | E8 | H33 | M585 | E9 | H33 |
| M586 | E10 | H33 | M587 | E11 | H33 | M588 | E12 | H33 |
| M589 | E13 | H33 | M590 | E14 | H33 | M591 | E15 | H33 |
| M592 | E16 | H33 | M593 | E17 | H33 | M594 | E18 | H33 |

If the composition of the invention is used as host material in the light emitting layer, the concentration of the electron-transporting host material of the formula (1) as described above or described as preferred in the composition of the invention or in the light-emitting layer of the device of the invention is in the range from 5% by weight to 90% by weight, preferably in the range from 10% by weight to 85% by weight, more preferably in the range from 20% by weight to 85% by weight, even more preferably in the range from 30% by weight to 80% by weight, very especially preferably in the range from 20% by weight to 60% by weight and most preferably in the range from 30% by weight to 50% by weight, based on the overall mixture or based on the overall composition of the light-emitting layer.

The concentration of the hole-transporting host material of the formula (2) or of the formula (3) or of the formula (4) or of the formula (5) as described above or described as preferred in the mixture of the invention or in the light-emitting layer of the device of the invention is in the range from 10% by weight to 95% by weight, preferably in the range from 15% by weight to 90% by weight, more preferably in the range from 15% by weight to 80% by weight, even more preferably in the range from 20% by weight to 70% by weight, very especially preferably in the range from 40% by weight to 80% by weight and most preferably in the range from 50% by weight to 70% by weight, based on the overall mixture or based on the overall composition of the light-emitting layer.

The present invention also relates to a mixture which, as well as the aforementioned host materials of the formula (1) and of the formula (2) or of the formula (3) or of the formula (4) or of the formula (5) as described above or described as preferred, especially mixtures M1 to M594, also contains at least one phosphorescent emitter. The present invention also relates to an organic electroluminescent device as described above or described as preferred, wherein the light-emitting layer, as well as the aforementioned host materials of the formula (1) and of the formula (2) or of the formula (3) or of the formula (4) or of the formula (5), as described above or described as preferred, especially the material combinations M1 to M594, also comprises at least one phosphorescent emitter.

The concentration of the phosphorescent emitter as described hereinafter or described as preferred in the mixture of the invention or in the light-emitting layer of the device of the invention is in the range from 1% by weight to 30% by weight, preferably in the range from 2% by weight to 20% by weight, more preferably in the range from 4% by weight to 15% by weight, even more preferably in the range from 8% by weight to 12% by weight, based on the overall mixture or based on the overall composition of the light-emitting layer.

The term “phosphorescent emitters” typically encompasses compounds where the light is emitted through a spin-forbidden transition from an excited state having higher spin multiplicity, i.e. a spin state >1, for example through a transition from a triplet state or a state having an even higher spin quantum number, for example a quintet state. This preferably means a transition from a triplet state.

Suitable phosphorescent emitters (=triplet emitters) are especially compounds which, when suitably excited, emit light, preferably in the visible region, and also contain at least one atom of atomic number greater than 20, preferably greater than 38 and less than 84, more preferably greater than 56 and less than 80, especially a metal having this atomic number. Preferred phosphorescence emitters used are compounds containing copper, molybdenum, tungsten, rhenium, ruthenium, osmium, rhodium, iridium, palladium, platinum, silver, gold or europium, especially compounds containing iridium or platinum. In the context of the present invention, all luminescent compounds containing the abovementioned metals are regarded as phosphorescent emitters.

In general, all phosphorescent complexes as used for phosphorescent OLEDs according to the prior art and as known to those skilled in the art in the field of organic electroluminescent devices are suitable.

Examples of the emitters described above can be found in applications WO 00/70655, WO 2001/41512, WO 2002/02714, WO 2002/15645, EP 1191613, EP 1191612, EP 1191614, WO 05/033244, WO 05/019373, US 2005/0258742, WO 2009/146770, WO 2010/015307, WO 2010/031485, WO 2010/054731, WO 2010/054728, WO 2010/086089, WO 2010/099852, WO 2010/102709, WO 2011/032626, WO 2011/066898, WO 2011/157339, WO 2012/007086, WO 2014/008982, WO 2014/023377, WO 2014/094961, WO 2014/094960, WO 2015/036074, WO 2015/104045, WO 2015/117718, WO 2016/015815, WO 2016/124304, WO 2017/032439, WO 2018/011186, WO 2018/001990, WO 2018/019687, WO 2018/019688, WO 2018/041769, WO 2018/054798, WO 2018/069196, WO 2018/069197, WO 2018/069273, WO 2018/178001, WO 2018/177981, WO 2019/020538, WO 2019/115423, WO 2019/158453 and WO 2019/179909.

Preferred phosphorescent emitters according to the present invention correspond to compounds of the formula (IIIa):

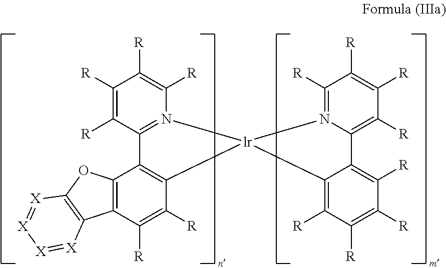

where the symbols and indices for this formula (IIIa) are defined as follows:
n′+m′ is 3, n′ is 1 or 2, m′ is 2 or 1,
X is N or CR,
R is H, D, CN, F, or a branched or linear alkyl group having 1 to 10 carbon atoms or a partly or fully deuterated branched or linear alkyl group having 1 to 10 carbon atoms or a cycloalkyl group having 4 to 7 carbon atoms or a partly or fully deuterated cycloalkyl group having 4 to 7 carbon atoms, or an aromatic ring system having 6 to 24 aromatic ring atoms or a heteroaromatic ring system having 5 to 24 aromatic ring atoms, which may be partly or fully deuterated.

The invention accordingly further provides an organic electroluminescent device as described above or described as preferred, characterized in that the light-emitting layer, as well as the host materials 1 and 2, comprises at least one phosphorescent emitter conforming to the formula (IIIa) as described above.

In emitters of the formula (IIIa), n is preferably 1 and m is preferably 2.

In emitters of the formula (IIIa), preferably one X is selected from N and the other X are CR, or all X are CR.

In emitters of the formula (IIIa), at least one R is preferably different from H or two R are different from H and have one of the other definitions given above for the emitters of the formula (IIIa).

In a further preferred embodiment of the compounds of the formula (IIIa), the compounds are partly or fully deuterated.

Further preferred phosphorescent emitters according to the present invention conform to the formulae (I), (II) and (III):

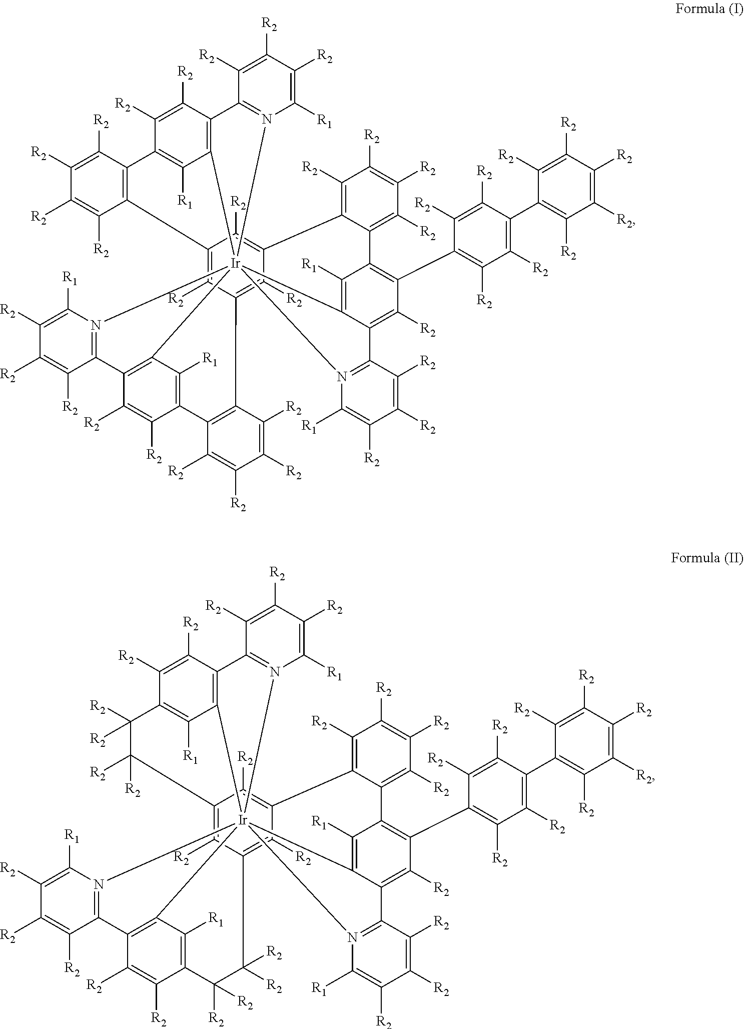

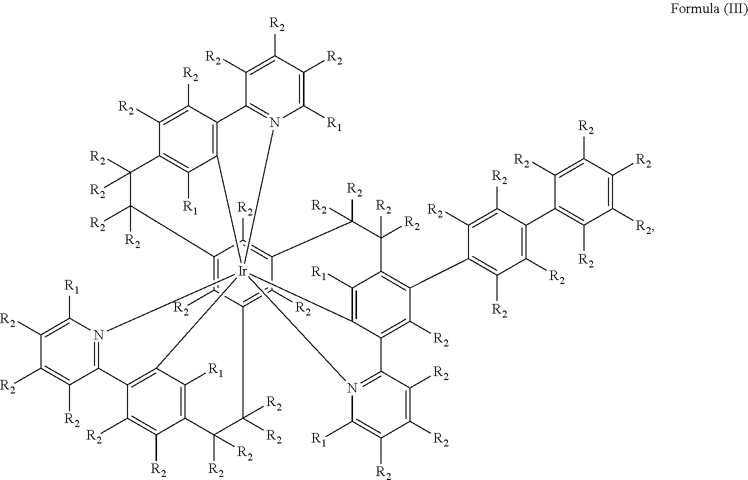

where the symbols and indices for these formulae (I), (II) and (III) are defined as follows:
R₁ is H or D, R² is H, D, or a branched or linear alkyl group having 1 to 10 carbon atoms or a partly or fully deuterated branched or linear alkyl group having 1 to 10 carbon atoms or a cycloalkyl group which has 4 to 10 carbon atoms and may be partly or fully substituted by deuterium.

Preferred phosphorescent emitters according to the present invention conform to the formulae (IV), (V) and (VI)

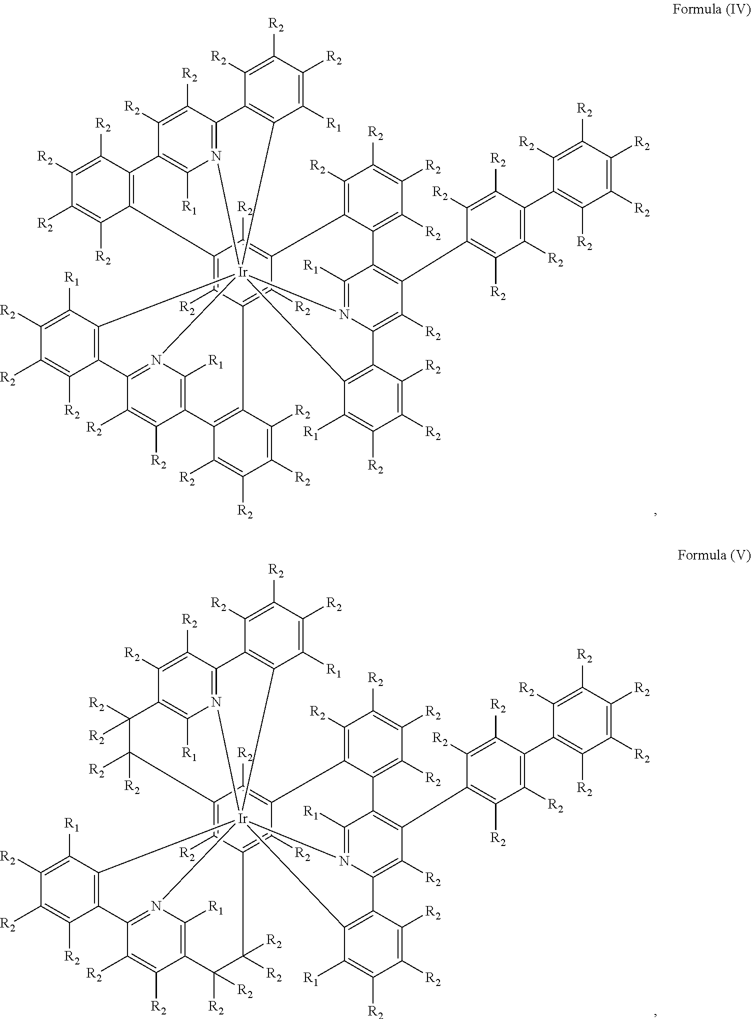

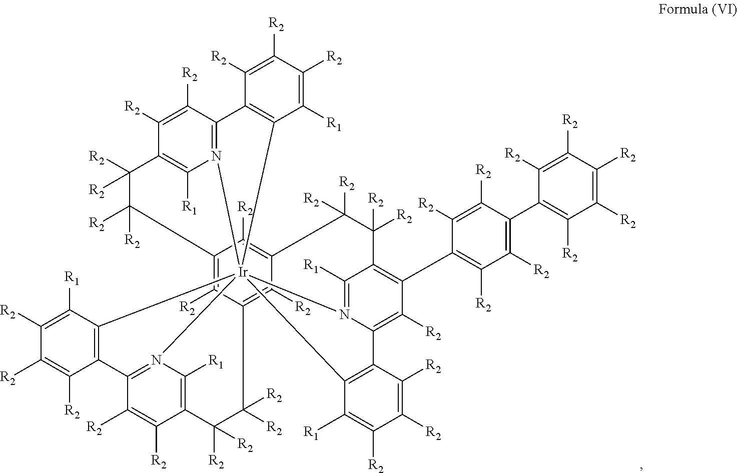

where the symbols and indices for these formulae (IV), (V) and (VI) are defined as follows:
R₁ is H or D, R² is H, D, F or a branched or linear alkyl group having 1 to 10 carbon atoms or a partly or fully deuterated branched or linear alkyl group having 1 to 10 carbon atoms or a cycloalkyl group which has 4 to 10 carbon atoms and may be partly or fully substituted by deuterium.

Preferred examples of phosphorescent emitters are listed in table 6 below.

TABLE 6

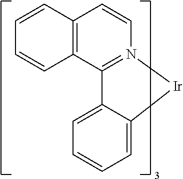

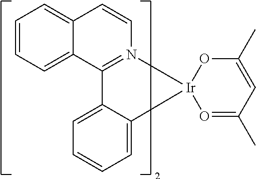

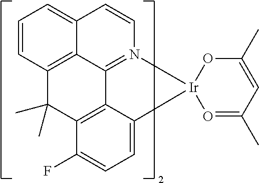

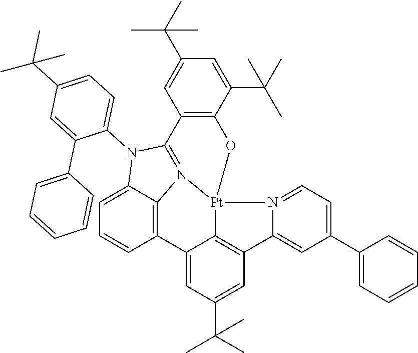

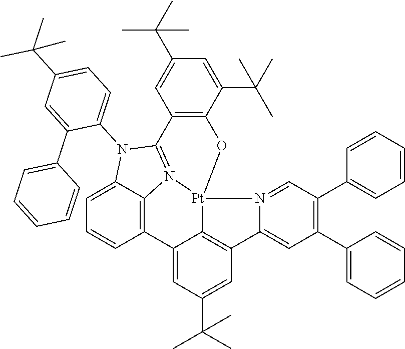

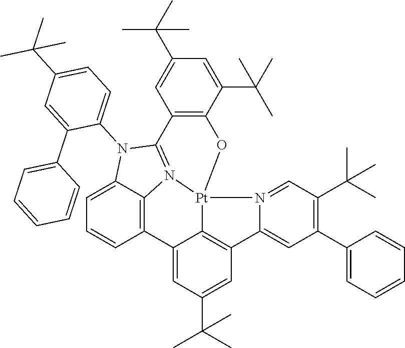

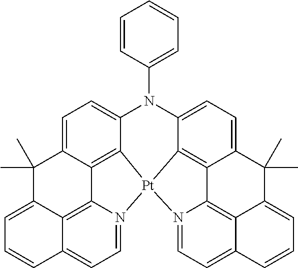

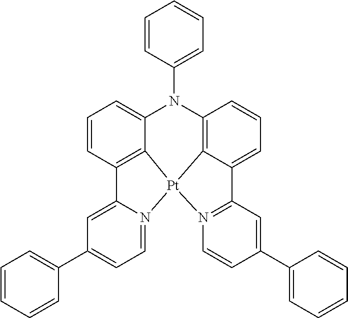

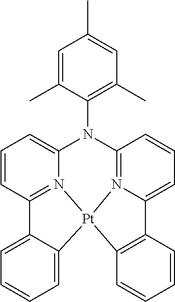

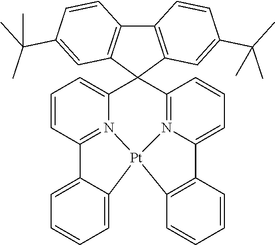

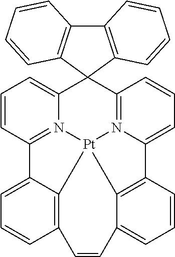

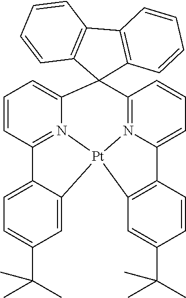

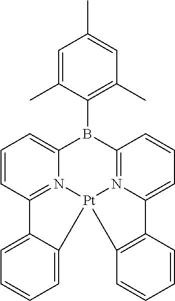

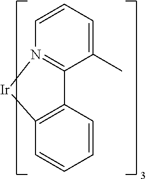

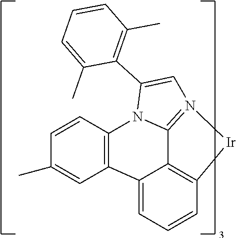

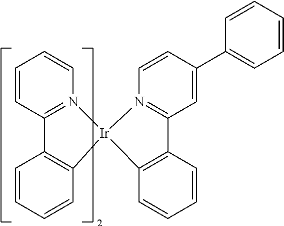

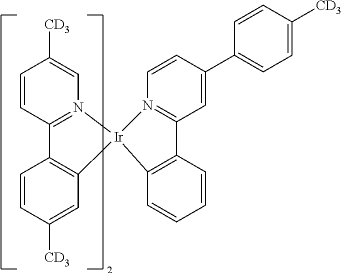

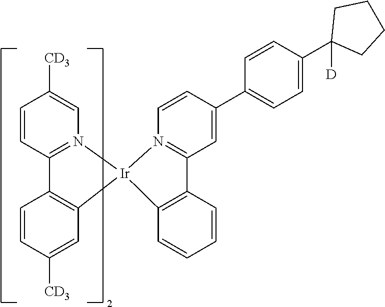

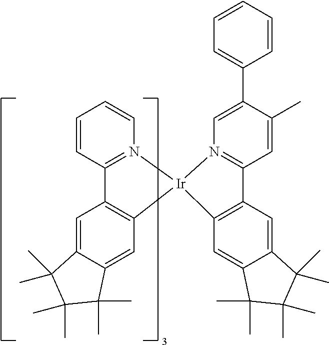

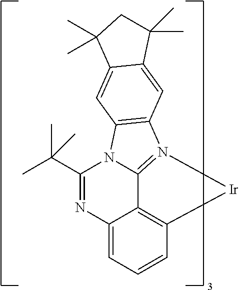

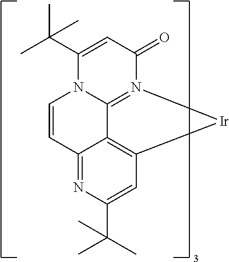

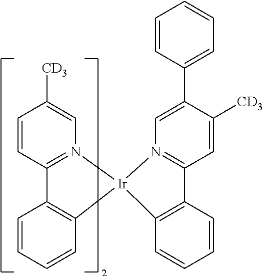

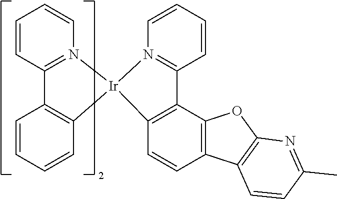

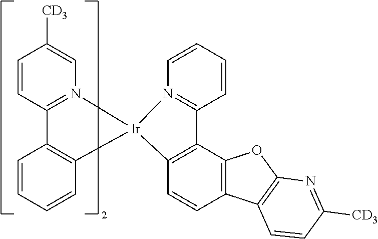

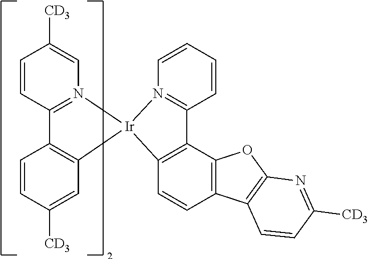

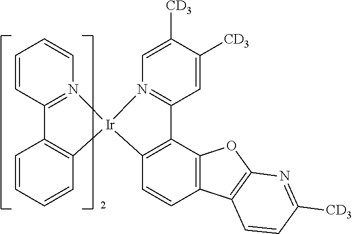

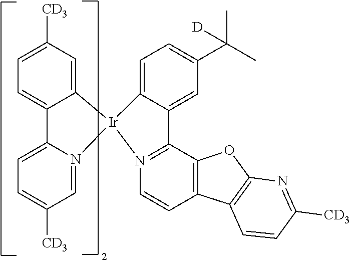

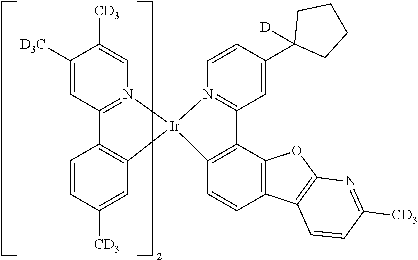

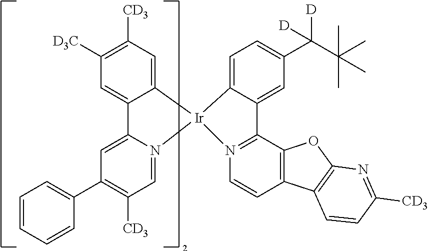

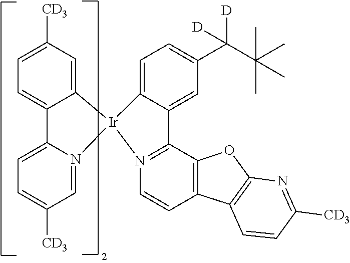

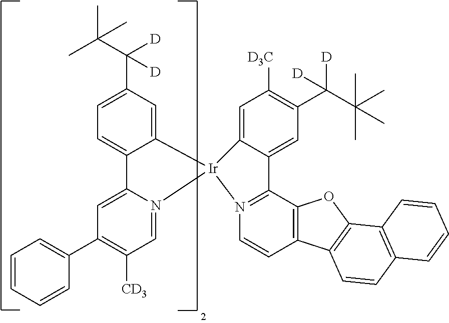

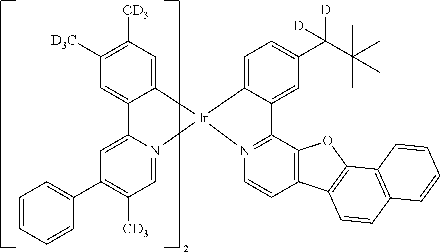

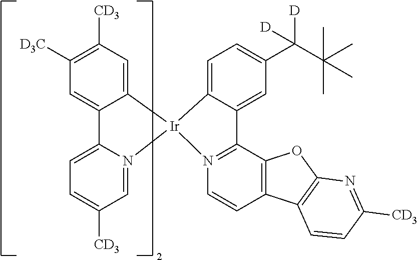

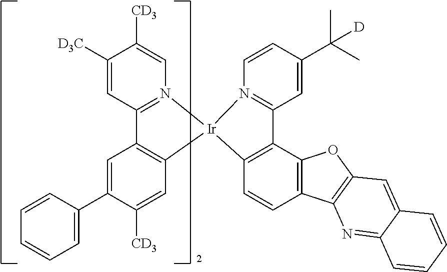

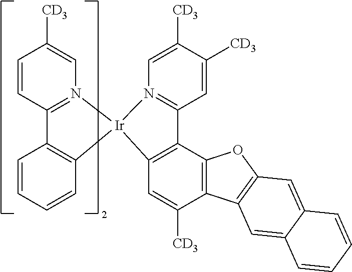

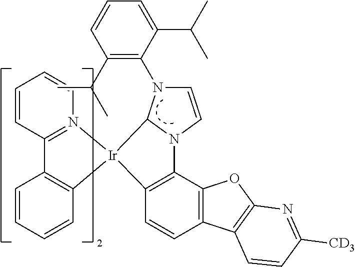

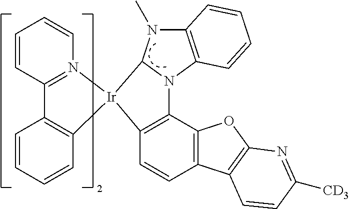

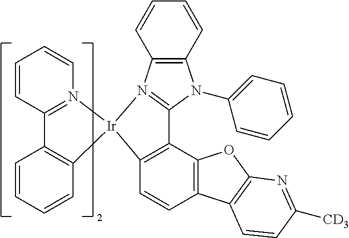

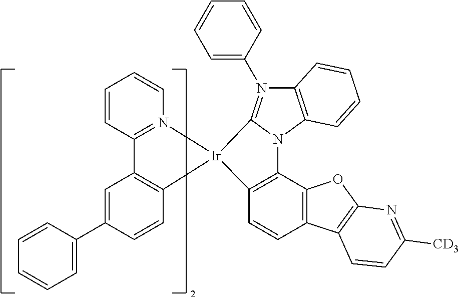

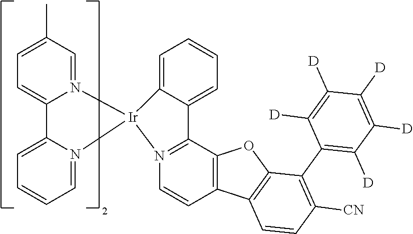

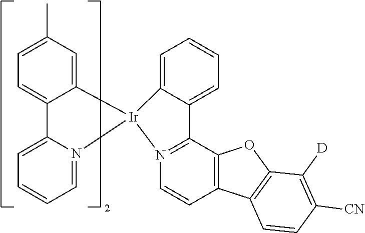

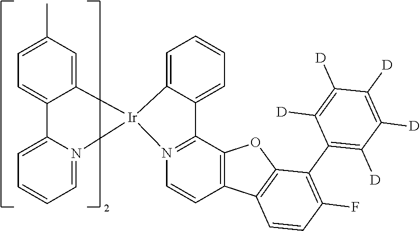

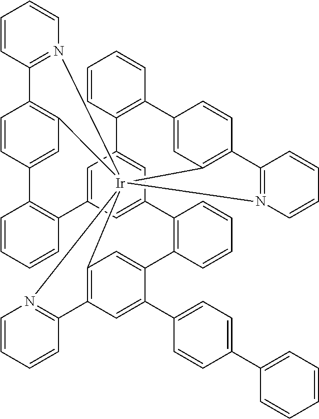

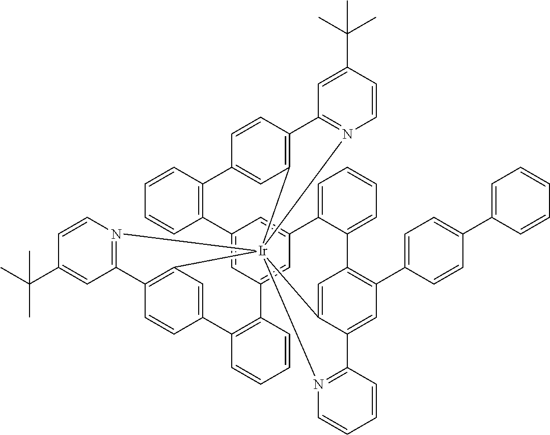

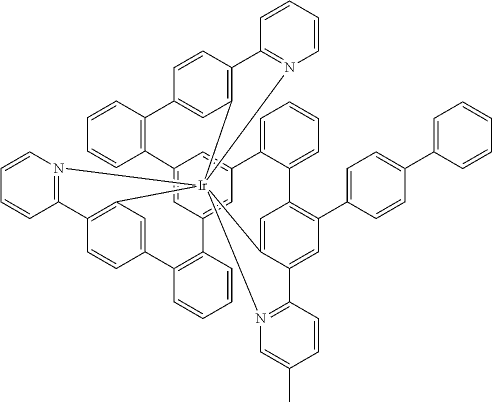

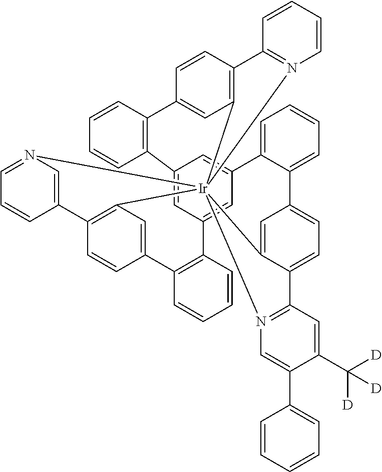

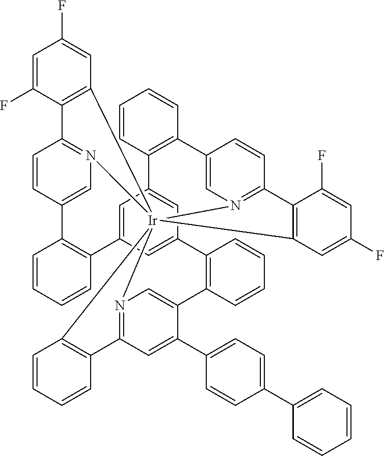

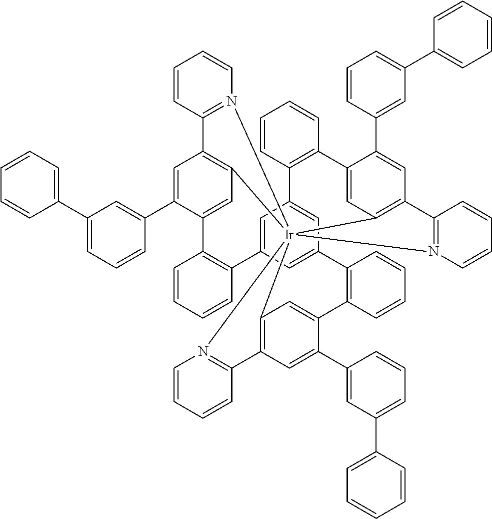

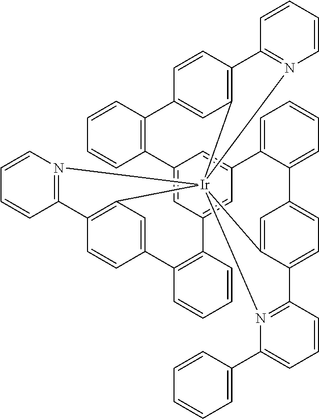

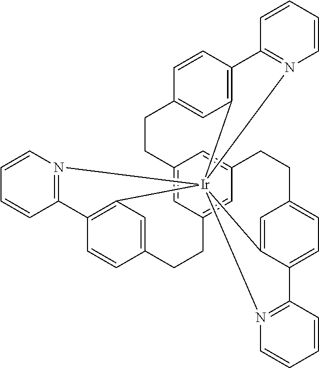

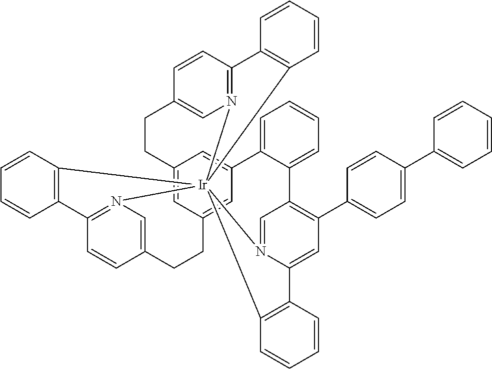

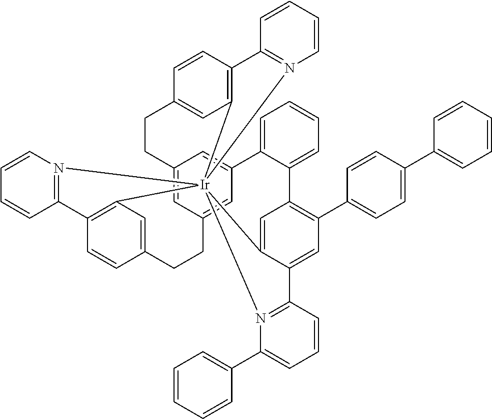

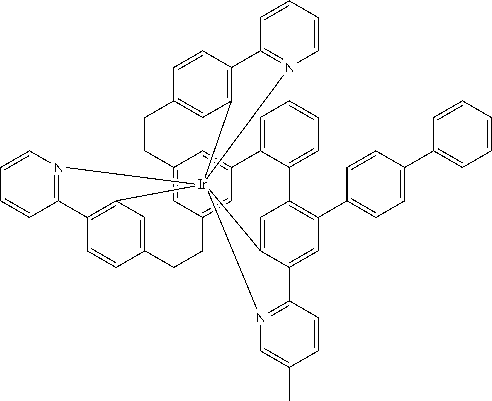

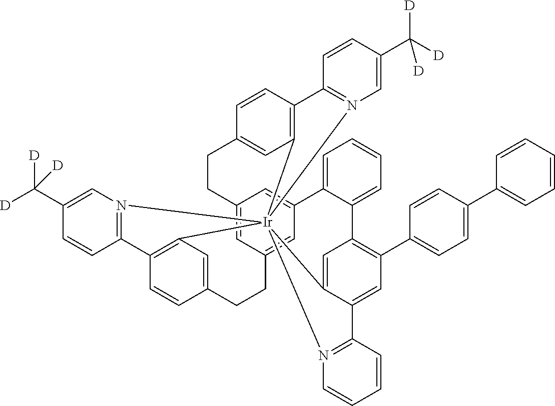

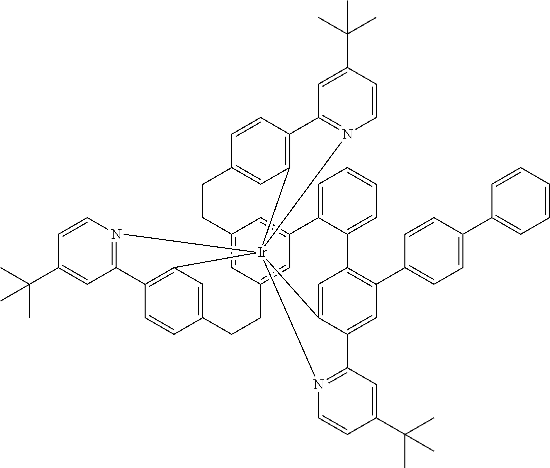

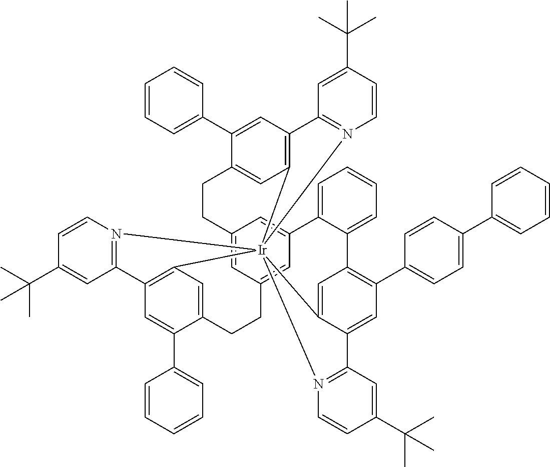

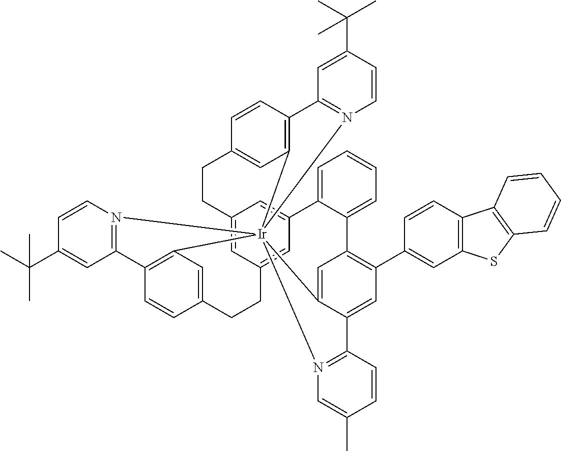

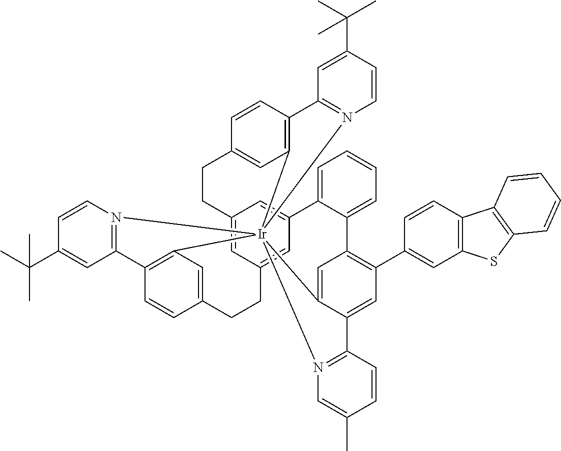

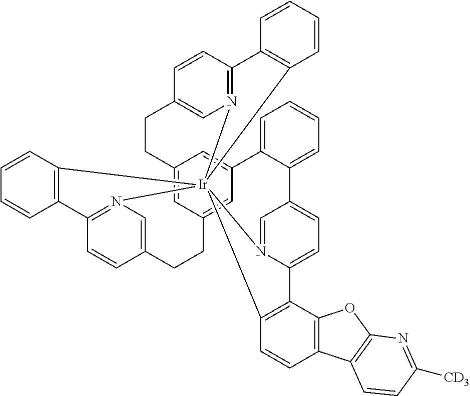

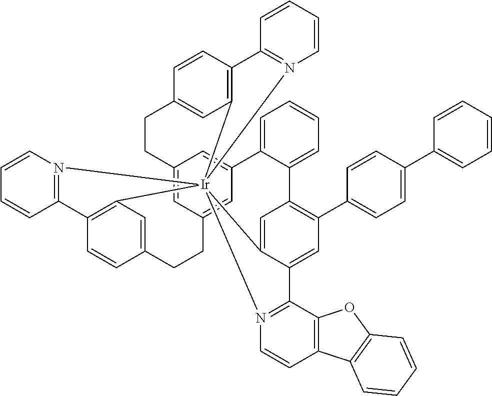

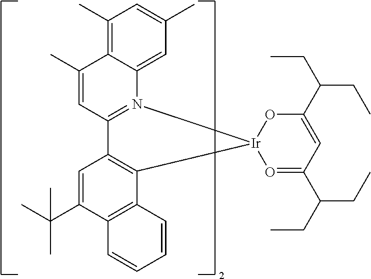

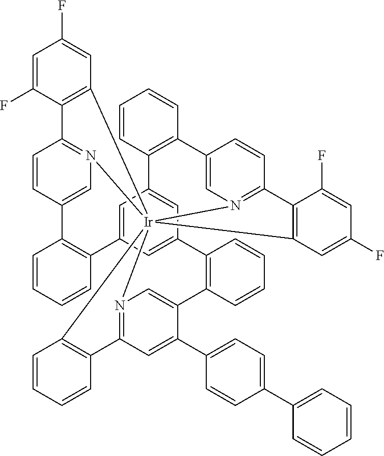

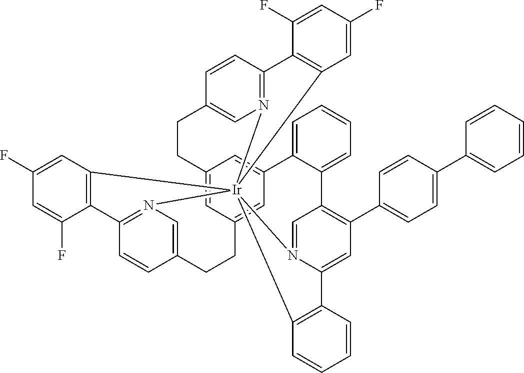

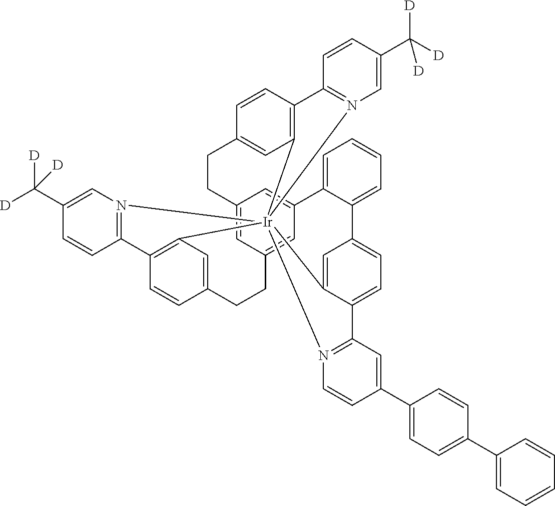

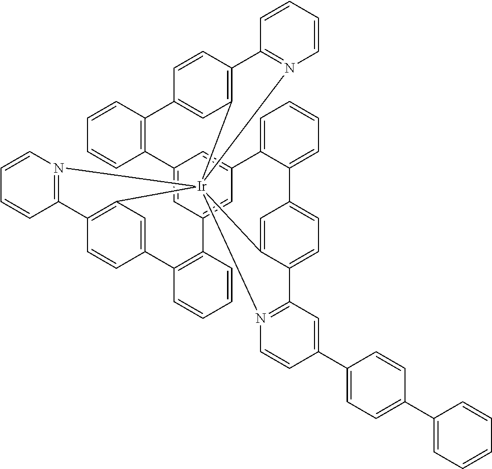

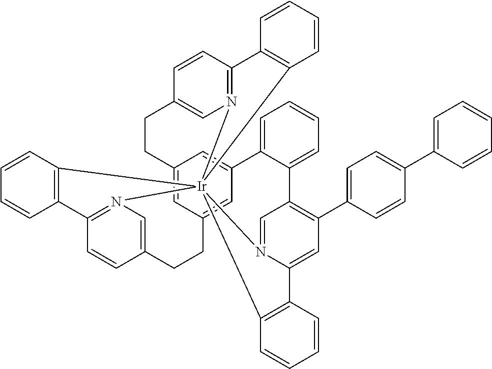

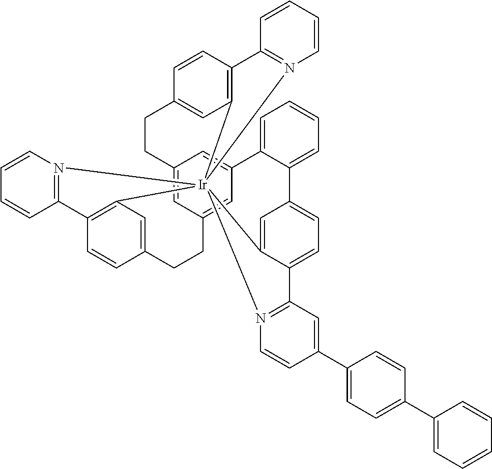

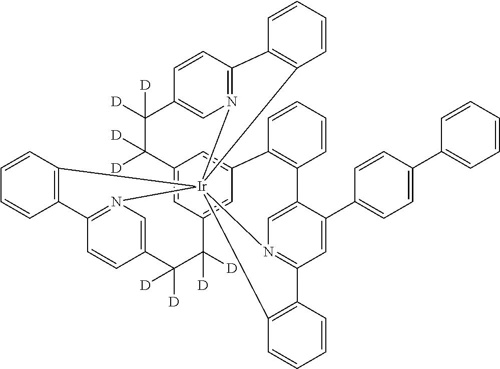

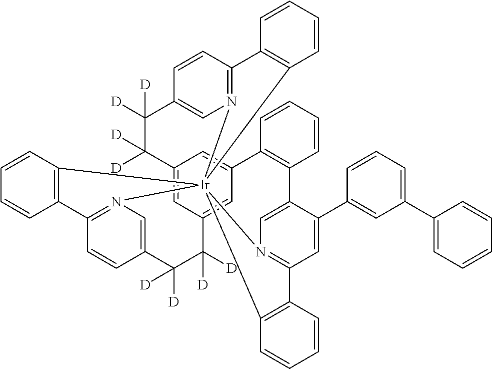

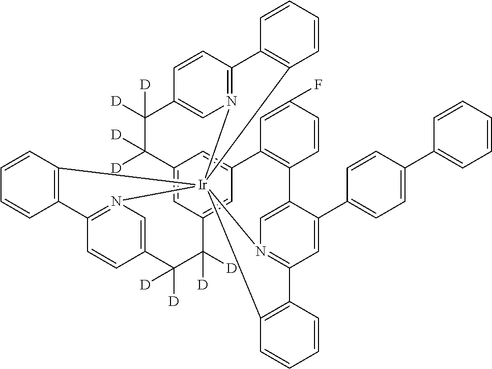

In the mixtures of the invention or in the light-emitting layer of the device of the invention, preferably every mixture M1, M2, M3, M4, M5, M6, M7, M8, M9, M10, M11, M12, M13, M14, M15, M16, M17, M18, M19, M20, M21, M22, M23, M24, M25, M26, M27, M28, M29, M30, M31, M32, M33, M34, M35, M36, M37, M38, M39, M40, M41, M42, M43, M44, M45, M46, M47, M48, M49, M50, M51, M52, M53, M54, M55, M56, M57, M58, M59, M60, M61, M62, M63, M64, M65, M66, M67, M68, M69, M70, M71, M72, M73, M74, M75, M76, M77, M78, M79, M80, M81, M82, M83, M84, M85, M86, M87, M88, M89, M90, M91, M92, M93, M94, M95, M96, M97, M98, M99, M100, M101, M102, M103, M104, M105, M106, M107, M108, M109, M110, M111, M112, M113, M114, M115, M116, M117, M118, M119, M120, M121, M122, M123, M124, M125, M126, M127, M128, M129, M130, M131, M132, M133, M134, M135, M136, M137, M138, M139, M140, M141, M142, M143, M144, M145, M146, M147, M148, M149, M150, M151, M152, M153, M154, M155, M156, M157, M158, M159, M160, M161, M162, M163, M164, M165, M166, M167, M168, M169, M170, M171, M172, M173, M174, M175, M176, M177, M178, M179, M180, M181, M182, M183, M184, M185, M186, M187, M188, M189, M190, M191, M192, M193, M194, M195, M196, M197, M198, M199, M200, M201, M202, M203, M204, M205, M206, M207, M208, M209, M210, M211, M212, M213, M214, M215, M216, M217, M218, M219, M220, M221, M222, M223, M224, M225, M226, M227, M228, M229, M230, M231, M232, M233, M234, M235, M236, M237, M238, M239, M240, M241, M242, M243, M244, M245, M246, M247, M248, M249, M250, M251, M252, M253, M254, M255, M256, M257, M258, M259, M260, M261, M262, M263, M264, M265, M266, M267, M268, M269, M270, M271, M272, M273, M274, M275, M276, M277, M278, M279, M280, M281, M282, M283, M284, M285, M286, M287, M288, M289, M290, M291, M292, M293, M294, M295, M296, M297, M298, M299, M300, M301, M302, M303, M304, M305, M306, M307, M308, M309, M310, M311, M312, M313, M314, M315, M316, M317, M318, M319, M320, M321, M322, M323, M324, M325, M326, M327, M328, M329, M330, M331, M332, M333, M334, M335, M336, M337, M338, M339, M340, M341, M342, M343, M344, M345, M346, M347, M348, M349, M350, M351, M352, M353, M354, M355, M356, M357, M358, M359, M360, M361, M362, M363, M364, M365, M366, M367, M368, M369, M370, M371, M372, M373, M374, M375, M376, M377, M378, M379, M380, M381, M382, M383, M384, M385, M386, M387, M388, M389, M390, M391, M392, M393, M394, M395, M396, M397, M398, M399, M400, M401, M402, M403, M404, M405, M406, M407, M408, M409, M410, M411, M412, M413, M414, M415, M416, M417, M418, M419, M420, M421, M422, M423, M424, M425, M426, M427, M428, M429, M430, M431, M432, M433, M434, M435, M436, M437, M438, M439, M440, M441, M442, M443, M444, M445, M446, M447, M448, M449, M450, M451, M452, M453, M454, M455, M456, M457, M458, M459, M460, M461, M462, M463, M464, M465, M466, M467, M468, M469, M470, M471, M472, M473, M474, M475, M476, M477, M478, M479, M480, M481, M482, M483, M484, M485, M486, M487, M488, M489, M490, M491, M492, M493, M494, M495, M496, M497, M498, M499, M500, M501, M502, M503, M504, M505, M506, M507, M508, M509, M510, M511, M512, M513, M514, M515, M516, M517, M518, M519, M520, M521, M522, M523, M524, M525, M526, M527, M528, M529, M530, M531, M532, M533, M534, M535, M536, M537, M538, M539, M540, M541, M542, M543, M544, M545, M546, M547, M548, M549, M550, M551, M552, M553, M554, M555, M556, M557, M558, M559, M560, M561, M562, M563, M564, M565, M566, M567, M568, M569, M570, M571, M572, M573, M574, M575, M576, M577, M578, M579, M580, M581, M582, M583, M584, M585, M586, M587, M588, M589, M590, M591, M592, M593, M594 is combined with a compound of the formula (IIIa) or a compound of the formulae (I) to (VI) or a compound from table 6.

The light-emitting layer in the organic electroluminescent device of the invention, comprising at least one phosphorescent emitter, is preferably an infrared-emitting or yellow-, orange-, red-, green-, blue- or ultraviolet-emitting layer, more preferably a yellow- or green- or red-emitting layer and most preferably a green- or red-emitting layer, especially a green-emitting layer.

What is meant here by a yellow-emitting layer is a layer having a photoluminescence maximum within the range from 540 to 570 nm. What is meant by an orange-emitting layer is a layer having a photoluminescence maximum within the range from 570 to 600 nm. What is meant by a red-emitting layer is a layer having a photoluminescence maximum within the range from 600 to 750 nm. What is meant by a green-emitting layer is a layer having a photoluminescence maximum within the range from 490 to 540 nm. What is meant by a blue-emitting layer is a layer having a photoluminescence maximum within the range from 440 to 490 nm. The photoluminescence maximum of the layer is determined here by measuring the photoluminescence spectrum of the layer having a layer thickness of 50 nm at room temperature, said layer containing the inventive combination of the host materials of the formulae (1) and (2) or of the formulae (1) and (3) or of the formulae (1) and (4) or of the formulae (1) and (5) and the appropriate emitter.

The photoluminescence spectrum of the layer is recorded, for example, with a commercial photoluminescence spectrometer.

The photoluminescence spectrum of the emitter chosen is generally measured in oxygen-free solution, 10⁻⁵ molar, at room temperature, a suitable solvent being any in which the chosen emitter dissolves in the concentration mentioned. Particularly suitable solvents are typically toluene or 2-methyl-THF, but also dichloromethane. Measurement is effected with a commercial photoluminescence spectrometer. The triplet energy T1 in eV is determined from the photoluminescence spectra of the emitters. First the peak maximum Plmax. (in nm) of the photoluminescence spectrum is determined. The peak maximum Plmax. (in nm) is then converted to eV by: E(T1 in eV)=1240/E(T1 in nm)=1240/PLmax. (in nm).

Preferred phosphorescent emitters are accordingly infrared emitters, preferably of the formula (IIIa), of the formulae (I) to (VI) or from table 6, the triplet energy T₁ of which is preferably ˜1.9 eV to ˜1.0 eV.

Preferred phosphorescent emitters are accordingly red emitters, preferably of the formula (IIIa), of the formulae (I) to (VI) or from table 6, the triplet energy T₁ of which is preferably ˜2.1 eV to ˜1.9 eV.

Preferred phosphorescent emitters are accordingly yellow emitters, preferably of the formula (IIIa), of the formulae (I) to (VI) or from table 6, the triplet energy T₁ of which is preferably ˜2.3 eV to ˜2.1 eV.

Preferred phosphorescent emitters are accordingly green emitters, preferably of the formula (IIIa), of the formulae (I) to (VI) or from table 6, the triplet energy T₁ of which is preferably ˜2.5 eV to ˜2.3 eV.

Preferred phosphorescent emitters are accordingly blue emitters, preferably of the formula (IIIa), of the formulae (I) to (VI) or from table 6, the triplet energy T₁ of which is preferably ˜3.1 eV to ˜2.5 eV.

Particularly preferred phosphorescent emitters are accordingly green or yellow emitters, preferably of the formula (IIIa), of the formulae (I) to (VI) or from table 6, as described above.

Very particularly preferred phosphorescent emitters are accordingly green emitters, preferably of the formula (IIIa), of the formulae (I) to (VI) or from table 6, the triplet energy T₁ of which is preferably ˜2.5 eV to ˜2.3 eV.

Most preferably, green emitters, preferably of the formula (IIIa), of the formulae (I) to (VI) or from table 6, as described above, are selected for the composition of the invention or emitting layer of the invention.

It is also possible for fluorescent emitters to be present in the light-emitting layer of the device of the invention.

Preferred fluorescent emitters are selected from the class of the arylamines. An arylamine or an aromatic amine in the context of this invention is understood to mean a compound containing three substituted or unsubstituted aromatic or heteroaromatic ring systems bonded directly to the nitrogen. Preferably, at least one of these aromatic or heteroaromatic ring systems is a fused ring system, more preferably having at least 14 ring atoms. Preferred examples of these are aromatic anthraceneamines, aromatic anthracenediamines, aromatic pyreneamines, aromatic pyrenediamines, aromatic chryseneamines or aromatic chrysenediamines. What is meant by an aromatic anthraceneamine is a compound in which a diarylamino group is bonded directly to an anthracene group, preferably in the 9 position. What is meant by an aromatic anthracenediamine is a compound in which two diarylamino groups are bonded directly to an anthracene group, preferably in the 9,10 positions. Aromatic pyreneamines, pyrenediamines, chryseneamines and chrysenediamines are defined analogously, where the diarylamino groups are bonded to the pyrene preferably in the 1 position or 1,6 positions. Further preferred fluorescent emitters are indenofluoreneamines or -diamines, for example according to WO 2006/108497 or WO 2006/122630, benzoindenofluoreneamines or -diamines, for example according to WO 2008/006449, and dibenzoindenofluoreneamines or -diamines, for example according to WO 2007/140847, and the indenofluorene derivatives having fused aryl groups disclosed in WO 2010/012328.

In a further preferred embodiment of the invention, the at least one light-emitting layer of the organic electroluminescent device, as well as the host materials 1 and 2 as described above or described as preferred, may comprise further host materials or matrix materials, called mixed matrix systems. The mixed matrix systems preferably comprise three or four different matrix materials, more preferably three different matrix materials (in other words, one further matrix component in addition to the host materials 1 and 2 as described above). Particularly suitable matrix materials which can be used in combination as matrix component in a mixed matrix system are selected from wide-band gap materials, bipolar host materials, electron transport materials (ETM) and hole transport materials (HTM).

What is meant herein by a wide-band gap material is a material within the scope of the disclosure of U.S. Pat. No. 7,294,849 which is characterized by a band gap of at least 3.5 eV, the band gap meaning the gap between the HOMO and LUMO energy of a material.

In one embodiment of the present invention, the mixture does not contain any further constituents, i.e. functional materials, aside from the constituents of electron-transporting host material of the formula (1) and hole-transporting host material of the formula (2) or of the formula (3) or of the formula (4) or of the formula (5). These are material mixtures that are used as such for production of the light-emitting layer. These mixtures are also referred to as premix systems that are used as the sole material source in the vapor deposition of the host materials for the light-emitting layer and have a constant mixing ratio in the vapor deposition. In this way, it is possible in a simple and rapid manner to achieve the vapor deposition of a layer with homogeneous distribution of the components without the need for precise actuation of a multitude of material sources.

In an alternative embodiment of the present invention, the mixture also comprises the phosphorescent emitter as described above, in addition to the constituents of electron-transporting host material of the formula (1) and hole-transporting host material of the formula (2) or of the formula (3) or of the formula (4) or of the formula (5). In the case of a suitable mixing ratio in the vapor deposition, this mixture may also be used as the sole material source as described above.

The components or constituents of the light-emitting layer of the device of the invention may thus be processed by vapor deposition or from solution. The material combination of host materials 1 and 2 as described above or described as preferred, optionally with the phosphorescent emitter as described above or described as preferred, are provided for that purpose in a formulation containing at least one solvent. These formulations may, for example, be solutions, dispersions or emulsions. For this purpose, it may be preferable to use mixtures of two or more solvents.

The present invention therefore further provides a formulation comprising an inventive mixture of host materials 1 and 2 as described above, optionally in combination with a phosphorescent emitter as described above or described as preferred, and at least one solvent. The formulation preferably contains at least one compound of the formula (1) and one compound of the formula (2) or of the formula (3) or of the formula (4) or of the formula (5) and a solvent. Additionally preferred is a process that the formulation containing at least one compound of the formula (1) and a compound of the formula (2) or of the formula (3) or of the formula (4) or of the formula (5) is used to apply the organic layer.

Suitable and preferred solvents are, for example, toluene, anisole, o-, m- or p-xylene, methyl benzoate, mesitylene, tetralin, veratrole, THF, methyl-THF, THP, chlorobenzene, dioxane, phenoxytoluene, especially 3-phenoxytoluene, (−)-fenchone, 1,2,3,5-tetramethylbenzene, 1,2,4,5-tetramethylbenzene, 1-methylnaphthalene, 2-methylbenzothiazole, 2-phenoxyethanol, 2-pyrrolidinone, 3-methylanisole, 4-methylanisole, 3,4-dimethylanisole, 3,5-dimethylanisole, acetophenone, α-terpineol, benzothiazole, butyl benzoate, cumene, cyclohexanol, cyclohexanone, cyclohexylbenzene, decalin, dodecylbenzene, ethyl benzoate, indane, methyl benzoate, NMP, p-cymene, phenetole, 1,4-diisopropylbenzene, dibenzyl ether, diethylene glycol butyl methyl ether, triethylene glycol butyl methyl ether, diethylene glycol dibutyl ether, triethylene glycol dimethyl ether, diethylene glycol monobutyl ether, tripropylene glycol dimethyl ether, tetraethylene glycol dimethyl ether, 2-isopropylnaphthalene, pentylbenzene, hexylbenzene, heptylbenzene, octylbenzene, 1,1-bis(3,4-dimethylphenyl)ethane, hexamethylindane or mixtures of these solvents.

The formulation here may also comprise at least one further organic or inorganic compound which is likewise used in the light-emitting layer of the device of the invention, especially a further emitting compound and/or a further matrix material.

The light-emitting layer in the device of the invention, according to the preferred embodiments and the emitting compound, contains preferably between 99.9% and 1% by volume, further preferably between 99% and 10% by volume, especially preferably between 98% and 60% by volume, very especially preferably between 97% and 80% by volume, of matrix material composed of at least one compound of the formula (1) and at least one compound of the formula (2) or of the formula (3) or of the formula (4) or of the formula (5) according to the preferred embodiments, based on the overall composition of emitter and matrix material. Correspondingly, the light-emitting layer in the device of the invention preferably contains between 0.1% and 99% by volume, further preferably between 1% and 90% by volume, more preferably between 2% and 40% by volume, most preferably between 3% and 20% by volume, of the emitter based on the overall composition of the light-emitting layer composed of emitter and matrix material. If the compounds are processed from solution, preference is given to using the corresponding amounts in % by weight rather than the above-specified amounts in % by volume.

The light-emitting layer in the device of the invention, according to the preferred embodiments and the emitting compound, preferably contains the matrix material of the formula (1) and the matrix material of the formula (2) or of the formula (3) or of the formula (4) or of the formula (5) in a percentage by volume ratio between 4:1 and 1:4, preferably between 1:3 and 1:1, more preferably between 1:2 and 1:1. If the compounds are processed from solution, preference is given to using the corresponding ratio in % by weight rather than the above-specified ratio in % by volume.

Preferred hole transport materials are materials that can be used in a hole transport, hole injection or electron blocker layer, such as indenofluoreneamine derivatives (for example according to WO 06/122630 or WO 06/100896), the amine derivatives disclosed in EP 1661888, hexaazatriphenylene derivatives (for example according to WO 01/049806), amine derivatives with fused aromatic systems (for example according to U.S. Pat. No. 5,061,569), the amine derivatives disclosed in WO 95/09147, monobenzoindenofluoreneamines (for example according to WO 08/006449), dibenzoindenofluoreneamines (for example according to WO 07/140847), dihydroacridine derivatives (e.g. WO 2012/150001).

The sequence of layers in the organic electroluminescent device of the invention is preferably as follows:

anode/hole injection layer/hole transport layer/electron blocker layer/emitting layer/hole blocker layer/electron transport layer/electron injection layer/cathode.

This sequence of the layers is a preferred sequence.

At the same time, it should be pointed out again that not all the layers mentioned need be present and/or that further layers may additionally be present.

The organic electroluminescent device of the invention may contain two or more emitting layers. Preferably, at least one of the emitting layers is the organic layer of the invention containing at least one compound of the formula (1) as host material 1 and at least one compound of the formula (2) or of the formula (3) or of the formula (4) or of the formula (5) as host material 2, as described above. More preferably, these emission layers in this case have several emission maxima between 380 nm and 750 nm overall, such that the overall result is white emission; in other words, various emitting compounds which may fluoresce or phosphoresce and which emit blue or yellow or orange or red light are used in the emitting layers. Especially preferred are three-layer systems, i.e. systems having three emitting layers, where the three layers show blue, green and orange or red emission (for the basic construction see, for example, WO 2005/011013). It should be noted that, for the production of white light, rather than a plurality of color-emitting emitter compounds, an emitter compound used individually which emits over a broad wavelength range may also be suitable.

Suitable charge transport materials as usable in the hole injection or hole transport layer or electron blocker layer or in the electron transport layer of the organic electroluminescent device of the invention are, for example, the compounds disclosed in Y. Shirota et al., Chem. Rev. 2007, 107(4), 953-1010, or other materials as used in these layers according to the prior art.

Materials used for the electron transport layer may be any materials as used according to the prior art as electron transport materials in the electron transport layer. Especially suitable are aluminum complexes, for example Alq₃, zirconium complexes, for example Zrq₄, benzimidazole derivatives, triazine derivatives, pyrimidine derivatives, pyridine derivatives, pyrazine derivatives, quinoxaline derivatives, quinoline derivatives, oxadiazole derivatives, aromatic ketones, lactams, boranes, diazaphosphole derivatives and phosphine oxide derivatives. Further suitable materials are derivatives of the abovementioned compounds as disclosed in JP 2000/053957, WO 2003/060956, WO 2004/028217, WO 2004/080975 and WO 2010/072300.

Suitable cathodes of the device of the invention are metals having a low work function, metal alloys or multilayer structures composed of various metals, for example alkaline earth metals, alkali metals, main group metals or lanthanoids (e.g. Ca, Ba, Mg, Al, In, Yb, Sm, etc.). Additionally suitable are alloys composed of an alkali metal or alkaline earth metal and silver, for example an alloy composed of magnesium and silver. In the case of multilayer structures, in addition to the metals mentioned, it is also possible to use further metals having a relatively high work function, for example Ag or Al, in which case combinations of the metals such as Ca/Ag, Mg/Ag or Ba/Ag, for example, are generally used. It may also be preferable to introduce a thin interlayer of a material having a high dielectric constant between a metallic cathode and the organic semiconductor. Examples of useful materials for this purpose are alkali metal or alkaline earth metal fluorides, but also the corresponding oxides or carbonates (e.g. LiF, Li₂O, BaF₂, MgO, NaF, CsF, Cs₂CO₃, etc.). It is also possible to use lithium quinolinate (LiQ) for this purpose. The layer thickness of this layer is preferably between 0.5 and 5 nm.

Preferred anodes are materials having a high work function. Preferably, the anode has a work function of greater than 4.5 eV versus vacuum. Firstly, metals having a high redox potential are suitable for this purpose, for example Ag, Pt or Au. Secondly, metal/metal oxide electrodes (e.g. Al/Ni/NiOₓ, Al/PtOₓ) may also be preferred. For some applications, at least one of the electrodes has to be transparent or partly transparent in order to enable either the irradiation of the organic material (organic solar cell) or the outcoupling of light (OLED, O-LASER). Preferred anode materials here are conductive mixed metal oxides. Particular preference is given to indium tin oxide (ITO) or indium zinc oxide (IZO). Preference is further given to conductive doped organic materials, especially conductive doped polymers. In addition, the anode may also consist of two or more layers, for example of an inner layer of ITO and an outer layer of a metal oxide, preferably tungsten oxide, molybdenum oxide or vanadium oxide.

The organic electroluminescent device of the invention, in the course of production, is appropriately (according to the application) structured, contact-connected and finally sealed, since the lifetime of the devices of the invention is shortened in the presence of water and/or air.

The production of the device of the invention is not restricted here. It is possible that one or more organic layers, including the light-emitting layer, are coated by a sublimation method. In this case, the materials are applied by vapor deposition in vacuum sublimation systems at an initial pressure of less than 10⁻⁵ mbar, preferably less than 10⁻⁶ mbar. In this case, however, it is also possible that the initial pressure is even lower, for example less than 10⁻⁷ mbar.

The organic electroluminescent device of the invention is preferably characterized in that one or more layers are coated by the OVPD (organic vapor phase deposition) method or with the aid of a carrier gas sublimation. In this case, the materials are applied at a pressure between 10⁻⁵ mbar and 1 bar. A special case of this method is the OVJP (organic vapor jet printing) method, in which the materials are applied directly by a nozzle and thus structured (for example M. S. Arnold et al., Appl. Phys. Lett. 2008, 92, 053301).

The organic electroluminescent device of the invention is further preferably characterized in that one or more organic layers comprising the composition of the invention are produced from solution, for example by spin-coating, or by any printing method, for example screen printing, flexographic printing, nozzle printing or offset printing, but more preferably LITI (light-induced thermal imaging, thermal transfer printing) or inkjet printing. For this purpose, soluble host materials 1 and 2 and phosphorescent emitters are needed. Processing from solution has the advantage that, for example, the light-emitting layer can be applied in a very simple and inexpensive manner. This technique is especially suitable for the mass production of organic electroluminescent devices.

In addition, hybrid methods are possible, in which, for example, one or more layers are applied from solution and one or more further layers are applied by vapor deposition.

These methods are known in general terms to those skilled in the art and can be applied to organic electroluminescent devices.

The invention therefore further provides a process for producing the organic electroluminescent device of the invention as described above or described as preferred, characterized in that the light-emitting layer is applied by gas phase deposition, especially by a sublimation method and/or by an OVPD (organic vapor phase deposition) method and/or with the aid of a carrier gas sublimation, or from solution, especially by spin-coating or by a printing method.

In the case of production by means of gas phase deposition, there are in principle two ways in which the light-emitting layer of the invention can be applied or vapor-deposited onto any substrate or the prior layer. Firstly, the materials used can each be initially charged in a material source and ultimately evaporated from the different material sources (“co-evaporation”). Secondly, the various materials can be premixed (premix systems) and the mixture can be initially charged in a single material source from which it is ultimately evaporated (“premix evaporation”). In this way, it is possible in a simple and rapid manner to achieve the vapor deposition of the light-emitting layer with homogeneous distribution of the components without the need for precise actuation of a multitude of material sources.

The invention accordingly further provides a process for producing the device of the invention, characterized in that the at least one compound of the formula (1) as described above or described as preferred and the at least one compound of the formula (2) or of the formula (3) or of the formula (4) or of the formula (5) as described above or described as preferred are deposited from the gas phase successively or simultaneously from at least two material sources, optionally with the at least one phosphorescent emitter as described above or described as preferred, and form the light-emitting layer.

In a preferred embodiment of the present invention, the light-emitting layer is applied by means of gas phase deposition, wherein the constituents of the composition are premixed and evaporated from a single material source.

The invention accordingly further provides a process for producing the device of the invention, characterized in that the at least one compound of the formula (1) and the at least one compound of the formula (2) or of the formula (3) or of the formula (4) or of the formula (5) are deposited from the gas phase as a mixture, successively or simultaneously with the at least one phosphorescent emitter, and form the light-emitting layer.

The invention further provides a process for producing the device of the invention as described above or described as preferred, characterized in that the at least one compound of the formula (1) and the at least one compound of the formula (2) or of the formula (3) or of the formula (4) or of the formula (5) as described above or described as preferred are applied from solution together with the at least one phosphorescent emitter in order to form the organic layer, which is preferably the light-emitting layer.

The devices of the invention feature the following surprising advantages over the prior art:

The use of the described material combination of the compounds of the formula (1) and compounds of the formulae (2) or of the formula (3) or of the formula (4) or of the formula (5), preferably as host material 1 and host material 2 in the light-emitting layer, as described above, leads in particular to an increase in the lifetime of the devices, with otherwise comparable performance data of the devices.

It should be pointed out that variations of the embodiments described in the present invention are covered by the scope of this invention. Any feature disclosed in the present invention may, unless this is explicitly ruled out, be exchanged for alternative features which serve the same purpose or an equivalent or similar purpose. Any feature disclosed in the present invention, unless stated otherwise, should therefore be considered as an example from a generic series or as an equivalent or similar feature.

All features of the present invention may be combined with one another in any manner, unless particular features and/or steps are mutually exclusive. This is especially true of preferred features of the present invention. Equally, features of non-essential combinations may be used separately (and not in combination).

The technical teaching disclosed with the present invention may be abstracted and combined with other examples.

The invention is illustrated in detail by the examples which follow, without any intention of restricting it thereby.

### Production of the OLEDs

Examples V1 to V6 and Ex1 to Ex23 which follow (see table 7) show the use of the material combinations of the invention in OLEDs.

Pretreatment for Examples V1 to Ex23: Glass plates coated with structured ITO (indium tin oxide) of thickness 50 nm are treated prior to coating, first with an oxygen plasma, followed by an argon plasma. These plasma-treated glass plates form the substrates to which the OLEDs are applied.

The OLEDs basically have the following layer structure: substrate/hole injection layer (HIL)/hole transport layer (HTL)/electron blocker layer (EBL)/emission layer (EML)/optional hole blocker layer (HBL)/electron transport layer (ETL)/optional electron injection layer (EIL) and finally a cathode. The cathode is formed by an aluminum layer of thickness 100 nm. The exact structure of the OLEDs can be found in table 7. The materials required for production of the OLEDs are shown in table 9. The device data of the OLEDs are listed in table 8. Examples V1-V6 are comparative examples with an electron-transporting and hole-transporting host according to the prior art specified in table 9. Examples Ex1 to Ex23 show data for OLEDs of the invention.

All materials are applied by thermal vapor deposition in a vacuum chamber. In this case, the emission layer always consists of at least one matrix material (host material), for the purposes of the invention at least two matrix materials, and an emitting dopant (emitter) which is added to the matrix material(s) in a particular proportion by volume by co-evaporation. Details given in such a form as E-C:SdT2:TEG2 (32%:61%:7%) mean here that the material E-C is present in the layer in a proportion by volume of 32%, the compound SdT2 as co-host in a proportion of 61%, and TEG2 in a proportion of 7%. Analogously, the electron transport layer may also consist of a mixture of two materials.

Electroluminescence spectra are determined at a luminance of 1000 cd/m², and these are used to calculate the CIE 1931 x and y color coordinates. The parameter U10 in table 8 refers to the voltage which is required for a current density of 10 mA/cm². EQE10 denotes the external quantum efficiency which is attained at 10 mA/cm². The lifetime LD is defined as the time after which luminance, measured in cd/m² in forward direction, drops from the starting luminance to a certain proportion L1 in the course of operation with constant current density j₀. A figure of L1=80% in table 8 means that the lifetime reported in the LD column corresponds to the time after which luminance in cd/m² falls to 80% of its starting value.

### Use of Mixtures of the Invention in OLEDs

The materials of the invention are used in examples Ex1 to Ex23 as matrix materials in the emission layer of green-phosphorescing OLEDs. As a comparison from the prior art, materials E-A, E-B, E-C and E-D are used in combination with the host materials SdT1, SdT2 and SdT3 in comparative examples V1 to V6. On comparison of the inventive examples with the corresponding comparative examples, it is clearly apparent that the inventive examples each show a distinct advantage in the lifetime of the OLEDs, with otherwise comparable performance data of the OLEDs.

TABLE 7

| | HIL | HTL | EBL | EML | HBL | ETL | EIL |
|---|---|---|---|---|---|---|---|
| Ex. | thickness | thickness | thickness | thickness | thickness | thickness | thickness |
| V1 | HTCN | SpMA1 | SpMA2 | E-C:SdT2:TEG2 | ST2 | ST2:LiQ | LiQ |
| | 5 nm | 230 nm | 20 nm | (31%:62%:7%) | 10 nm | (50%:50%) | 1 nm |
| | | | | 40 nm | | 30 nm | |
| V2 | HTCN | SpMA1 | SpMA2 | E-D:SdT3:TEG2 | ST2 | ST2:LiQ | LiQ |
| | 5 nm | 230 nm | 20 nm | (31%:62%:7%) | 10 nm | (50%:50%) | 1 nm |
| | | | | 40 nm | | 30 nm | |
| V3 | HTCN | SpMA1 | SpMA2 | E-B:SdT1:TEG2 | ST2 | ST2:LiQ | LiQ |
| | 5 nm | 230 nm | 20 nm | (31%:62%:7%) | 10 nm | (50%:50%) | 1 nm |
| | | | | 40 nm | | 30 nm | |
| V4 | HTCN | SpMA1 | SpMA2 | E-A:SdT1:TEG2 | ST2 | ST2:LiQ | LiQ |
| | 5 nm | 230 nm | 20 nm | (31%:62%:7%) | 10 nm | (50%:50%) | 1 nm |
| | | | | 40 nm | | 30 nm | |
| V5 | HTCN | SpMA1 | SpMA2 | E-C:SdT3:TEG2 | ST2 | ST2:LiQ | LiQ |
| | 5 nm | 230 nm | 20 nm | (31%:62%:7%) | 10 nm | (50%:50%) | 1 nm |
| | | | | 40 nm | | 30 nm | |
| V6 | HTCN | SpMA1 | SpMA2 | E-D:SdT3:TEG2 | ST2 | ST2:LiQ | LiQ |
| | 5 nm | 230 nm | 20 nm | (31%:62%:7%) | 10 nm | (50%:50%) | 1 nm |
| | | | | 40 nm | | 30 nm | |
| Ex1 | HTCN | SpMA1 | SpMA2 | E12:H10:TEG2 | ST2 | ST2:LiQ | LiQ |
| | 5 nm | 230 nm | 20 nm | (31%:62%:7%) | 10 nm | (50%:50%) | 1 nm |
| | | | | 40 nm | | 30 nm | |
| Ex2 | HTCN | SpMA1 | SpMA2 | E12:H4:TEG2 | ST2 | ST2:LiQ | LiQ |
| | 5 nm | 230 nm | 20 nm | (21%:72%:7%) | 10 nm | (50%:50%) | 1 nm |
| | | | | 40 nm | | 30 nm | |
| Ex3 | HTCN | SpMA1 | SpMA2 | E12:H25:TEG2 | ST2 | ST2:LiQ | LiQ |
| | 5 nm | 230 nm | 20 nm | (31%:62%:7%) | 5 nm | (50%:50%) | 1 nm |
| | | | | 40 nm | | 30 nm | |
| Ex4 | HTCN | SpMA1 | SpMA2 | E12:H27:TEG2 | ST2 | ST2:LiQ | LiQ |
| | 5 nm | 215 nm | 20 nm | (41%:52%:7%) | 5 nm | (50%:50%) | 1 nm |
| | | | | 40 nm | | 30 nm | |
| Ex5 | HTCN | SpMA1 | SpMA2 | E12:H16:TEG2 | ST2 | ST2:LiQ | LiQ |
| | 5 nm | 215 nm | 20 nm | (31%:62%:7%) | 5 nm | (50%:50%) | 1 nm |
| | | | | 40 nm | | 30 nm | |
| Ex6 | HTCN | SpMA1 | SpMA2 | E12:H20:TEG2 | ST2 | ST2:LiQ | LiQ |
| | 5 nm | 215 nm | 20 nm | (41%:52%:7%) | 5 nm | (50%:50%) | 1 nm |
| | | | | 40 nm | | 30 nm | |
| Ex7 | HTCN | SpMA1 | SpMA2 | E12:H20:TEG3 | ST2 | ST2:LiQ | LiQ |
| | 5 nm | 215 nm | 20 nm | (41%:52%:7%) | 5 nm | (50%:50%) | 1 nm |
| | | | | 40 nm | | 30 nm | |
| Ex8 | HTCN | SpMA1 | SpMA2 | E10:H28:TEG2 | ST2 | ST2:LiQ | LiQ |
| | 5 nm | 215 nm | 20 nm | (31%:62%:7%) | 5 nm | (50%:50%) | 1 nm |
| | | | | 40 nm | | 30 nm | |
| Ex9 | HTCN | SpMA1 | SpMA2 | E11:H3:TEG2 | ST2 | ST2:LiQ | LiQ |
| | 5 nm | 215 nm | 20 nm | (31%:62%:7%) | 5 nm | (50%:50%) | 1 nm |
| | | | | 40 nm | | 30 nm | |
| Ex10 | HTCN | SpMA1 | SpMA2 | E12:H18:TEG2 | ST2 | ST2:LiQ | LiQ |
| | 5 nm | 215 nm | 20 nm | (31%:62%:7%) | 5 nm | (50%:50%) | 1 nm |
| | | | | 40 nm | | 30 nm | |
| Ex11 | HTCN | SpMA1 | SpMA2 | E12:H29:TEG3 | ST2 | ST2:LiQ | LiQ |
| | 5 nm | 215 nm | 20 nm | (41%:52%:7%) | 5 nm | (50%:50%) | 1 nm |
| | | | | 40 nm | | 30 nm | |
| Ex12 | HTCN | SpMA1 | SpMA2 | E12:H11:TEG2 | ST2 | ST2:LiQ | LiQ |
| | 5 nm | 215 nm | 20 nm | (31%:62%:7%) | 5 nm | (50%:50%) | 1 nm |
| | | | | 40 nm | | 30 nm | |
| Ex13 | HTCN | SpMA1 | SpMA2 | E-C:H11:TEG2 | ST2 | ST2:LiQ | LiQ |
| | 5 nm | 215 nm | 20 nm | (31%:62%:7%) | 5 nm | (50%:50%) | 1 nm |
| | | | | 40 nm | | 30 nm | |
| Ex14 | HTCN | SpMA1 | SpMA2 | E-B:H12:TEG2 | ST2 | ST2:LiQ | LiQ |
| | 5 nm | 215 nm | 20 nm | (31%:62%:7%) | 5 nm | (50%:50%) | 1 nm |
| | | | | 40 nm | | 30 nm | |
| Ex15 | HTCN | SpMA1 | SpMA2 | E15:H8:TEG2 | ST2 | ST2:LiQ | LiQ |
| | 5 nm | 215 nm | 20 nm | (31%:62%:7%) | 5 nm | (50%:50%) | 1 nm |
| | | | | 40 nm | | 30 nm | |
| Ex16 | HTCN | SpMA1 | SpMA2 | E14:H23:TEG2 | ST2 | ST2:LiQ | LiQ |
| | 5 nm | 215 nm | 20 nm | (41%:52%:7%) | 5 nm | (50%:50%) | 1 nm |
| | | | | 40 nm | | 30 nm | |
| Ex17 | HTCN | SpMA1 | SpMA2 | E2:H26:TEG2 | ST2 | ST2:LiQ | LiQ |
| | 5 nm | 215 nm | 20 nm | (31%:62%:7%) | 5 nm | (50%:50%) | 1 nm |
| | | | | 40 nm | | 30 nm | |
| Ex18 | HTCN | SpMA1 | SpMA2 | E8:H33:TEG2 | ST2 | ST2:LiQ | LiQ |
| | 5 nm | 215 nm | 20 nm | (51%:42%:7%) | 5 nm | (50%:50%) | 1 nm |
| | | | | 40 nm | | 30 nm | |
| Ex19 | HTCN | SpMA1 | SpMA2 | E5:H15:TEG2 | ST2 | ST2:LiQ | LiQ |
| | 5 nm | 230 nm | 20 nm | (31%:62%:7%) | 10 nm | (50%:50%) | 1 nm |
| | | | | 40 nm | | 30 nm | |
| Ex20 | HTCN | SpMA1 | SpMA2 | E6:H13:TEG2 | ST2 | ST2:LiQ | LiQ |
| | 5 nm | 230 nm | 20 nm | (31%:62%:7%) | 10 nm | (50%:50%) | 1 nm |
| | | | | 40 nm | | 30 nm | |
| Ex21 | HTCN | SpMA1 | SpMA2 | E9:H5:TEG2 | ST2 | ST2:LiQ | LiQ |
| | 5 nm | 230 nm | 20 nm | (31%:62%:7%) | 10 nm | (50%:50%) | 1 nm |
| | | | | 40 nm | | 30 nm | |
| Ex22 | HTCN | SpMA1 | SpMA2 | E12:H13:TEG2 | ST2 | ST2:LiQ | LiQ |
| | 5 nm | 230 nm | 20 nm | (31%:62%:7%) | 10 nm | (50%:50%) | 1 nm |
| | | | | 40 nm | | 30 nm | |
| Ex23 | HTCN | SpMA1 | SpMA2 | E4:H30:TEG2 | ST2 | ST2:LiQ | LiQ |
| | 5 nm | 230 nm | 20 nm | (31%:62%:7%) | 10 nm | (50%:50%) | 1 nm |
| | | | | 40 nm | | 30 nm | |

TABLE 8
Data of the OLEDs

| | U10 | | CIE x/y at | J₀ | L1 | LT |
|---|---|---|---|---|---|---|
| Ex. | (V) | EQE10 | 1000 cd/cm² | (mA/cm²) | (%) | (h) |
| V1 | 4.4 | 22.5 | 0.34/0.63 | 40 | 80 | 550 |
| V2 | 4.2 | 22.9 | 0.35/0.63 | 40 | 80 | 405 |
| V3 | 4.4 | 22.1 | 0.34/0.64 | 40 | 80 | 750 |
| V4 | 4.4 | 22.4 | 0.34/0.64 | 40 | 80 | 520 |
| V5 | 4.6 | 22.7 | 0.34/0.64 | 40 | 80 | 510 |
| V6 | 4.4 | 22.3 | 0.35/0.63 | 40 | 80 | 390 |
| Ex1 | 4.3 | 23.1 | 0.34/0.63 | 40 | 80 | 980 |
| Ex2 | 4.6 | 22.2 | 0.34/0.63 | 40 | 80 | 1215 |
| Ex3 | 4.3 | 23.3 | 0.34/0.63 | 40 | 80 | 1540 |
| Ex4 | 4.2 | 22.8 | 0.33/0.62 | 40 | 80 | 1115 |
| Ex5 | 4.0 | 22.3 | 0.35/0.64 | 40 | 80 | 1210 |
| Ex6 | 4.1 | 22.6 | 0.34/0.63 | 40 | 80 | 845 |
| Ex7 | 4.9 | 20.4 | 0.34/0.63 | 40 | 80 | 770 |
| Ex8 | 4.2 | 22.6 | 0.35/0.64 | 40 | 80 | 1350 |
| Ex9 | 4.4 | 22.7 | 0.34/0.63 | 40 | 80 | 1020 |
| Ex10 | 4.2 | 22.1 | 0.34/0.63 | 40 | 80 | 1250 |
| Ex11 | 4.5 | 22.8 | 0.34/0.63 | 40 | 80 | 1140 |
| Ex12 | 4.3 | 22.1 | 0.34/0.63 | 40 | 80 | 1370 |
| Ex13 | 4.2 | 22.2 | 0.35/0.64 | 40 | 80 | 1260 |
| Ex14 | 4.5 | 22.6 | 0.34/0.63 | 40 | 80 | 1155 |
| Ex15 | 4.5 | 22.4 | 0.35/0.64 | 40 | 80 | 1530 |
| Ex16 | 4.4 | 22.4 | 0.34/0.63 | 40 | 80 | 820 |
| Ex17 | 4.4 | 22.6 | 0.34/0.63 | 40 | 80 | 1290 |
| Ex18 | 4.0 | 22.0 | 0.34/0.64 | 40 | 80 | 900 |
| Ex19 | 4.6 | 21.9 | 0.35/0.64 | 40 | 80 | 1190 |
| Ex20 | 4.6 | 23.2 | 0.34/0.63 | 40 | 80 | 1150 |
| Ex21 | 4.4 | 22.5 | 0.34/0.63 | 40 | 80 | 915 |
| Ex22 | 4.2 | 22.7 | 0.34/0.63 | 40 | 80 | 1235 |
| Ex23 | 4.3 | 23.0 | 0.35/0.64 | 40 | 80 | 920 |

TABLE 9
Structural formulae of the materials in the OLEDs

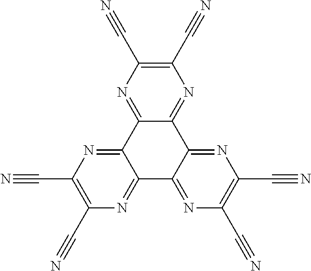 [SMILES: N#Cc1nc2c3nc(C#N)c(C#N)nc3c3nc(C#N)c(C#N)nc3c2nc1C#N]

HTCN

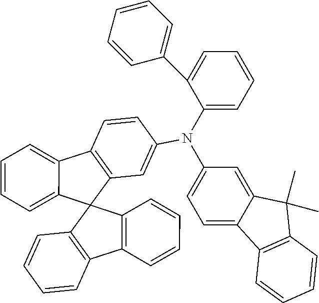 [SMILES: CC1(C)c2ccccc2-c2ccc(N(c3ccc4c(c3)C3(c5ccccc5-c5ccccc53)c3ccccc3-4)c3ccccc3-c3ccccc3)cc21]

SpMA1

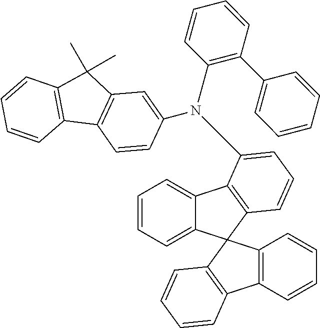 [SMILES: CC1(C)c2ccccc2-c2ccc(N(c3ccccc3-c3ccccc3)c3cccc4c3-c3ccccc3C43c4ccccc4-c4ccccc43)cc21]

SpMA2

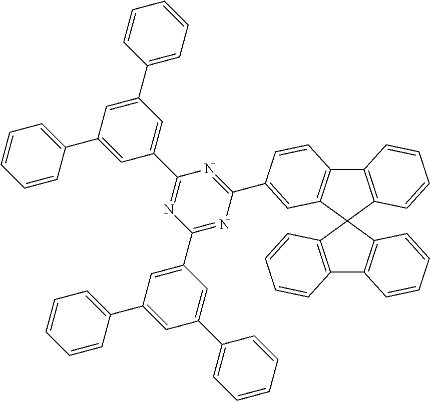 [SMILES: c1ccc(-c2cc(-c3ccccc3)cc(-c3nc(-c4cc(-c5ccccc5)cc(-c5ccccc5)c4)nc(-c4ccc5c(c4)C4(c6ccccc6-c6ccccc64)c4ccccc4-5)n3)c2)cc1]

ST2

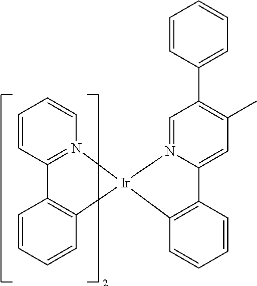

TEG1

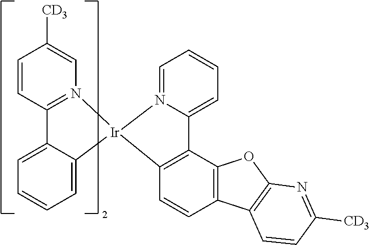

TEG2

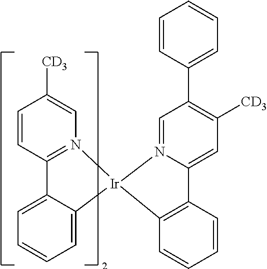

TEG3

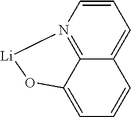

LiQ

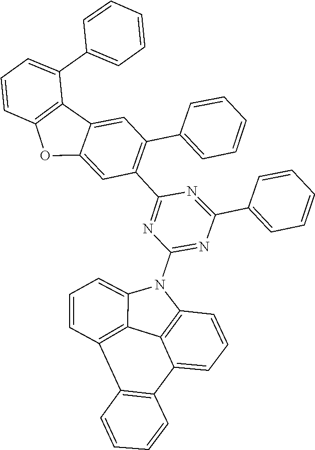 [SMILES: c1ccc(-c2nc(-c3cc4oc5cccc(-c6ccccc6)c5c4cc3-c3ccccc3)nc(-n3c4cccc5c6ccccc6c6cccc3c6c54)n2)cc1]

E-A CN112961145

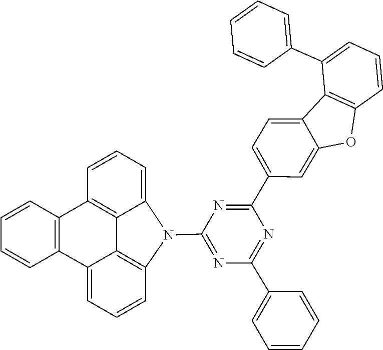 [SMILES: c1ccc(-c2nc(-c3ccc4c(c3)oc3cccc(-c5ccccc5)c34)nc(-n3c4cccc5c6ccccc6c6cccc3c6c54)n2)cc1]

E-B CN112961145

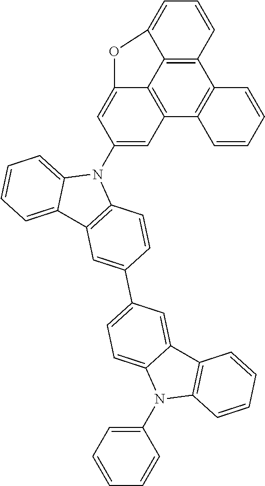 [SMILES: c1ccc(-n2c3ccccc3c3cc(-c4ccc5c(c4)c4ccccc4n5-c4cc5oc6cccc7c8ccccc8c(c4)c5c67)ccc32)cc1]

SdT1 CN112961145

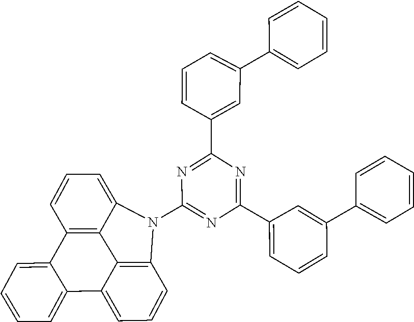 [SMILES: c1ccc(-c2cccc(-c3nc(-c4cccc(-c5ccccc5)c4)nc(-n4c5cccc6c7ccccc7c7cccc4c7c65)n3)c2)cc1]

E-C WO2012048781

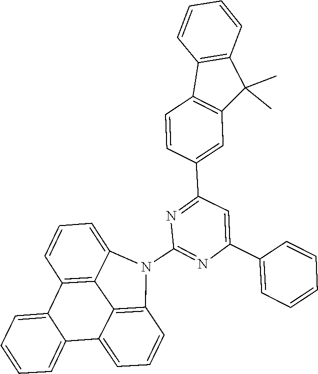 [SMILES: CC1(C)c2ccccc2-c2ccc(-c3cc(-c4ccccc4)nc(-n4c5cccc6c7ccccc7c7cccc4c7c65)n3)cc21]

E-D WO2012048781

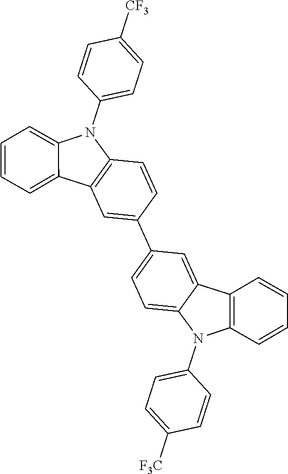 [SMILES: FC(F)(F)c1ccc(-n2c3ccccc3c3cc(-c4ccc5c(c4)c4ccccc4n5-c4ccc(C(F)(F)F)cc4)ccc32)cc1]

SdT2 JP 2004/288381
mentioned in WO2012048781

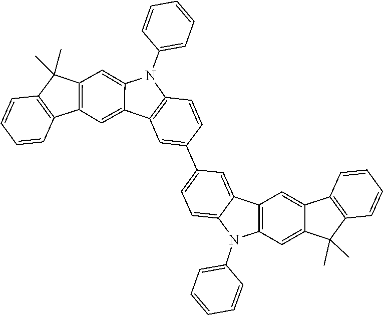 [SMILES: CC1(C)c2ccccc2-c2cc3c4cc(-c5ccc6c(c5)c5cc7c(cc5n6-c5ccccc5)C(C)(C)c5ccccc5-7)ccc4n(-c4ccccc4)c3cc21]

SdT3 - WO2010/136109 mentioned in
WO2012048781

The syntheses which follow, unless stated otherwise, are conducted under a protective gas atmosphere in dried solvents. The solvents and reagents can be purchased, for example, from Sigma-ALDRICH or ABCR. The respective figures in square brackets or the numbers quoted for individual compounds relate to the CAS numbers of the compounds known from the literature.

### Preparation of the Compounds

### a) Synthesis of the Precursors

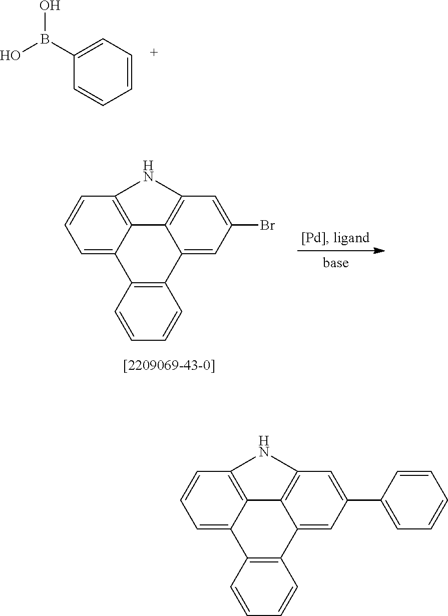 [SMILES: OB(O)c1ccccc1 ; Brc1cc2[nH]c3cccc4c5ccccc5c(c1)c2c34 ; c1ccc(-c2cc3[nH]c4cccc5c6ccccc6c(c2)c3c45)cc1]

25.6 g (210 mmol; 1.00 eq.) of phenylboronic acid, 81 g (255 mmol; 1.21 eq.) of 2-phenyl-4H-naphtho[1,2,3,4-def]carbazole and 44.5 g (420 mmol, 2.00 eq.) of sodium carbonate [CAS 497-19-8] are suspended in a mixture of 1000 ml of dioxane [CAS 123-91-1], 1000 ml of toluene [CAS 108-88-3] and 400 ml of water. To this suspension is added 4.85 g (4.20 mmol; 0.02 eq.) of tetrakis(triphenylphosphine)palladium(0) [CAS 14221-01-3], and the reaction mixture is heated under reflux for 16 h. After cooling, the organic phase is removed, filtered 10 through silica gel, washed three times with 200 ml of water and then concentrated to dryness. The yield is 38 g (121 mmol; 79% of theory).

The following compounds can be obtained analogously:

| No. | Reactant 1 | Product | Yield |
|---|---|---|---|
| 1a | 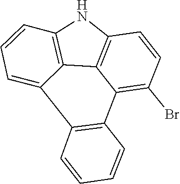 [SMILES: Brc1ccc2[nH]c3cccc4c5ccccc5c1c2c34] | 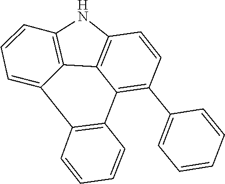 [SMILES: c1ccc(-c2ccc3[nH]c4cccc5c6ccccc6c2c3c45)cc1] | 81% |
| | [2637505-49-6] | | |
| 2a | 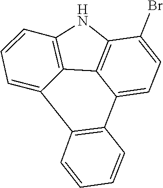 [SMILES: Brc1ccc2c3ccccc3c3cccc4[nH]c1c2c43] | 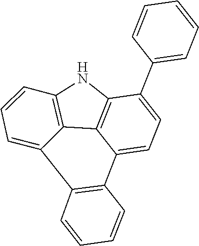 [SMILES: c1ccc(-c2ccc3c4ccccc4c4cccc5[nH]c2c3c54)cc1] | 63% |
| | [2209068-40-4] | | |
| 3a | 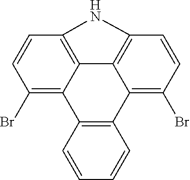 [SMILES: Brc1ccc2[nH]c3ccc(Br)c4c5ccccc5c1c2c34] | 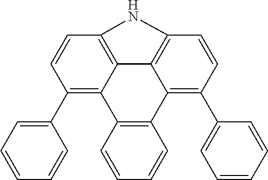 [SMILES: c1ccc(-c2ccc3[nH]c4ccc(-c5ccccc5)c5c6ccccc6c2c3c45)cc1] | 57% |

b) Synthesis of the Products with Reference to Example E12

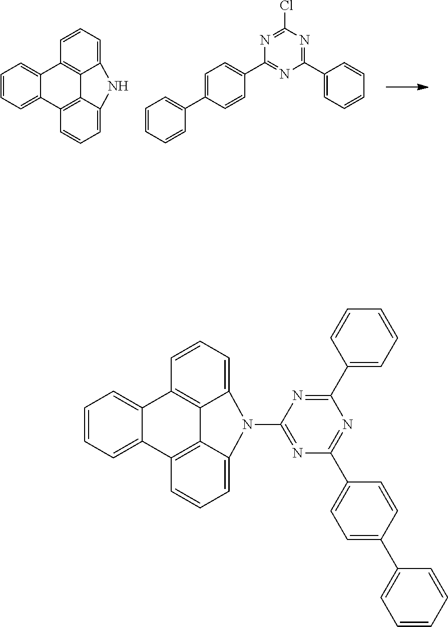

An initial charge of 19-azapentacyclo[14.2.1.05,¹8.06,¹¹.0^{12,1}7]nonadeca-1(18),2,4,6,8,10,12,14,16-nonaene (12.15 g) in tetrahydrofuran (120 ml) is cooled to −5° C. Subsequently, the mixture is cooled, 24.5 ml of n-hexyllithium (33% 2.47 mol/l in hexane) is added, and the mixture is stirred at −5° C. for a further 1 h. Subsequently, a solution of 2-{[1,1′-biphenyl]-4-yl}-4-chloro-6-phenyl-1,3,5-triazine (19.9 g) and tetrahydrofuran (245 ml) is added and the mixture is stirred at −5° C. for a further 1.5 h. The mixture is allowed to come to room temperature overnight, and then 500 ml of MeOH is added. The precipitated solids are filtered off with suction and washed with n-heptane. This is followed by basic hot extraction three times with o-xylene over aluminum oxide and then sublimation twice under high vacuum.

Yield: 14.36 g (64%) of solid material, purity >99.9% by HPLC.

The compounds that follow can be obtained analogously. Workup and purification can also be accomplished using other standard solvents and purification methods.

| No. | Reactant 1 | Reactant 2 | Product | Yield |
|---|---|---|---|---|
| E11 | 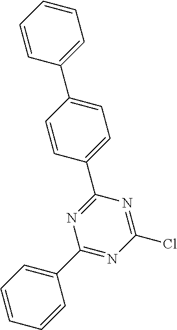 [SMILES: Clc1nc(-c2ccccc2)nc(-c2ccc(-c3ccccc3)cc2)n1] | 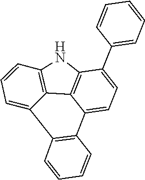 [SMILES: c1ccc(-c2ccc3c4ccccc4c4cccc5[nH]c2c3c54)cc1] | 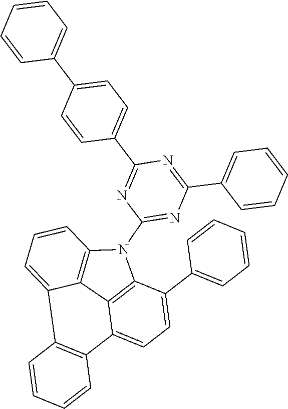 [SMILES: c1ccc(-c2ccc(-c3nc(-c4ccccc4)nc(-n4c5cccc6c7ccccc7c7ccc(-c8ccccc8)c4c7c65)n3)cc2)cc1] | 60% |
| | [1472062-94-4] | | | |
| E5 | 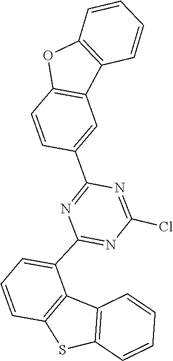 [SMILES: Clc1nc(-c2ccc3oc4ccccc4c3c2)nc(-c2cccc3sc4ccccc4c23)n1] | 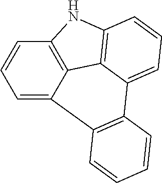 [SMILES: c1ccc2c(c1)c1cccc3[nH]c4cccc2c4c31] | 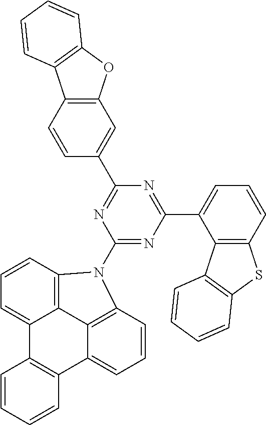 [SMILES: c1ccc2c(c1)oc1cc(-c3nc(-c4cccc5sc6ccccc6c45)nc(-n4c5cccc6c7ccccc7c7cccc4c7c65)n3)ccc12] | 70% |
| | | [109606-75-9] | | |
| E10 | 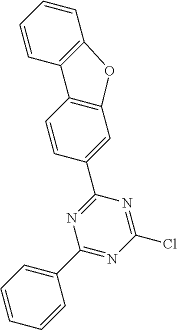 [SMILES: Clc1nc(-c2ccccc2)nc(-c2ccc3c(c2)oc2ccccc23)n1] | 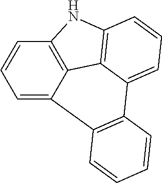 [SMILES: c1ccc2c(c1)c1cccc3[nH]c4cccc2c4c31] | 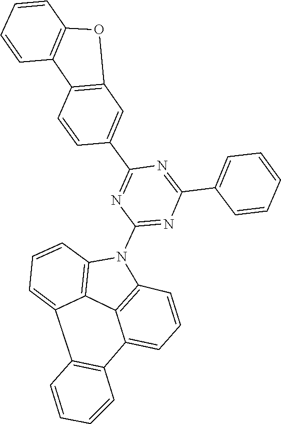 [SMILES: c1ccc(-c2nc(-c3ccc4c(c3)oc3ccccc34)nc(-n3c4cccc5c6ccccc6c6cccc3c6c54)n2)cc1] | 66% |
| | [2142681-84-1] | [109606-75-9] | | |
| E6 | 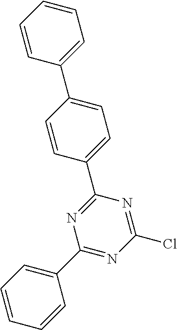 [SMILES: Clc1nc(-c2ccccc2)nc(-c2ccc(-c3ccccc3)cc2)n1] | 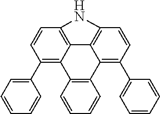 [SMILES: c1ccc(-c2ccc3[nH]c4ccc(-c5ccccc5)c5c6ccccc6c2c3c45)cc1] | 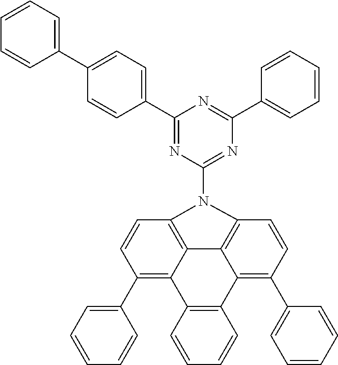 [SMILES: c1ccc(-c2ccc(-c3nc(-c4ccccc4)nc(-n4c5ccc(-c6ccccc6)c6c7ccccc7c7c(-c8ccccc8)ccc4c7c65)n3)cc2)cc1] | 60% |
| | [1472062-94-4] | | | |
| E13 | 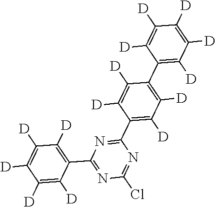 [SMILES: [2H]c1c([2H])c([2H])c(-c2nc(Cl)nc(-c3c([2H])c([2H])c(-c4c([2H])c([2H])c([2H])c([2H])c4[2H])c([2H])c3[2H])n2)c([2H])c1[2H]] | 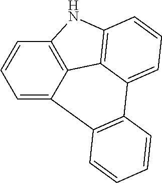 [SMILES: c1ccc2c(c1)c1cccc3[nH]c4cccc2c4c31] | 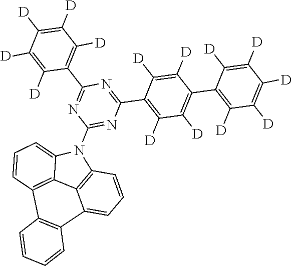 [SMILES: [2H]c1c([2H])c([2H])c(-c2nc(-c3c([2H])c([2H])c(-c4c([2H])c([2H])c([2H])c([2H])c4[2H])c([2H])c3[2H])nc(-n3c4cccc5c6ccccc6c6cccc3c6c54)n2)c([2H])c1[2H]] | 58% |
| | | [109606-75-9] | | |
| E9 | 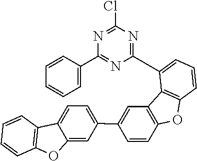 [SMILES: Clc1nc(-c2ccccc2)nc(-c2cccc3oc4ccc(-c5ccc6c(c5)oc5ccccc56)cc4c23)n1] | 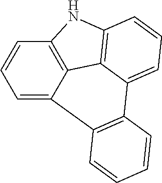 [SMILES: c1ccc2c(c1)c1cccc3[nH]c4cccc2c4c31] | 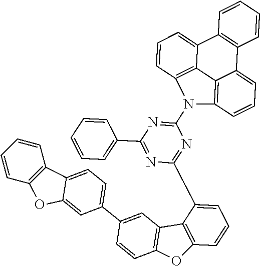 [SMILES: c1ccc(-c2nc(-c3cccc4oc5ccc(-c6ccc7c(c6)oc6ccccc67)cc5c34)nc(-n3c4cccc5c6ccccc6c6cccc3c6c54)n2)cc1] | 52% |
| | | [109606-75-9] | | |
| E4 | 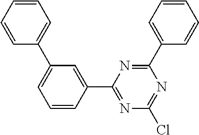 [SMILES: Clc1nc(-c2ccccc2)nc(-c2cccc(-c3ccccc3)c2)n1] | 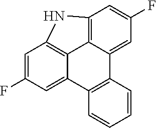 [SMILES: Fc1cc2[nH]c3cc(F)cc4c5ccccc5c(c1)c2c34] | 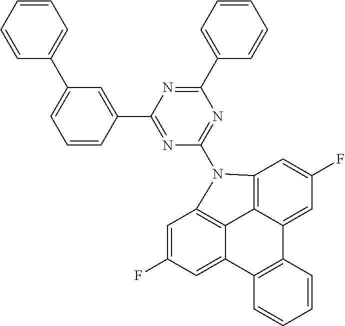 [SMILES: Fc1cc2c3ccccc3c3cc(F)cc4c3c2c(c1)n4-c1nc(-c2ccccc2)nc(-c2cccc(-c3ccccc3)c2)n1] | 42% |

1.-12. (canceled)

13. A composition comprising at least one compound of the formula (1) and at least one compound of the formula (2), of the formula (3), of the formula (4) or of the formula (5): 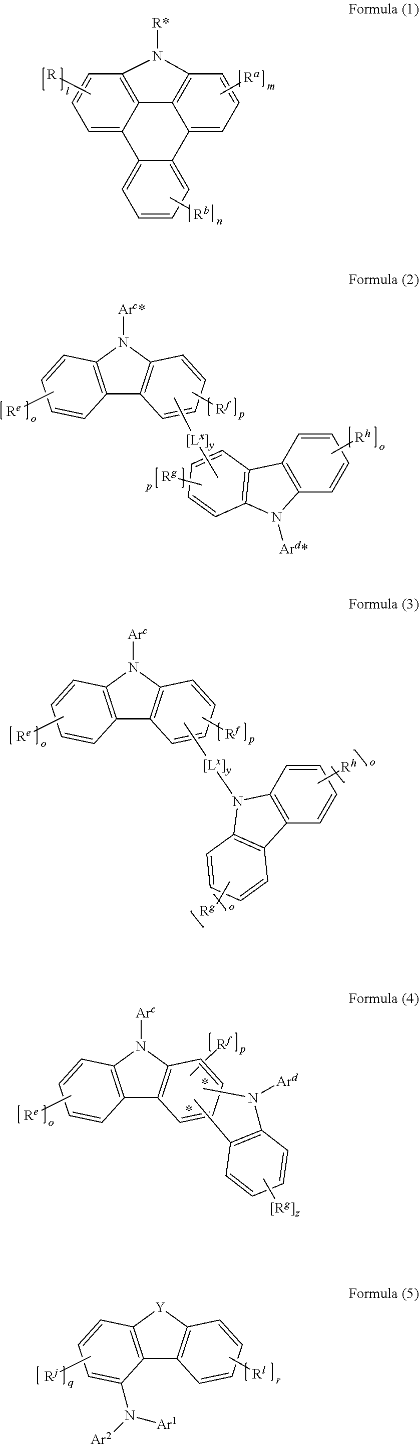
wherein
R* is a group of the following formula (1a): 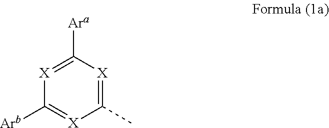
where the dashed bond represents the bond to the nitrogen atom in formula (1);
X is the same or different at each instance and is N or CRE, with the proviso that at least one X group is N and, if X is CR^{c}, this does not form a ring with Ar^{a} or Ar^{b};
Ar^{a}, Ar^{b} are the same or different at each instance and are each independently an aromatic ring system having 6 to 40 aromatic ring atoms or a heteroaromatic ring system having 5 to 40 aromatic ring atoms, each of which may be substituted by one or more R¹ radicals;
L^{x} is the same or different at each instance and is an aromatic ring system having 6 to 40 aromatic ring atoms or a heteroaromatic ring system having 5 to 40 aromatic ring atoms, which may be substituted in each case by one or more R¹ radicals;
Ar^{c}, Ar^{d} are the same or different at each instance and are each independently an aromatic ring system having 6 to 40 aromatic ring atoms or a heteroaromatic ring system having 5 to 40 aromatic ring atoms, each of which may be substituted by one or more R^{d} radicals;
Ar^{c*}, Ar^{d*} are the same or different at each instance and are each independently an aromatic ring system which has 6 to 40 aromatic ring atoms and may be substituted in each case by one or more R^{d} radicals or a heteroaryl group selected from the group consisting of dibenzofuran, dibenzothiophene, carbazole, triphenyleno[1,2-bcd]thiophene, substituted naphtho[1,2,3,4,def]carbazole, phenoxazine, phenothiazine, indolo[3,2,1-jk]carbazole, biscarbazole, benzcarbazole, indenocarbazole, indolocarbazole, benzofurocarbazole, benzothioenocarbazole, dihydroacridine, dihydrophenazine, dibenzodioxin, thianthrene, phenoxathiine, each of which may be substituted by one or more R^{d} radicals, where, independently of one another, an R^{f} radical or an R^{e} radical together with an Ar^{c*} radical or an R^{g} radical or an R^{h} radical together with an Ar^{d*} radical may form a monocyclic or polycyclic, aliphatic, aromatic or heteroaromatic ring, or a group of the formula (6a) or (6b), where the dashed bond represents the bond to the nitrogen atom in the formula (2); 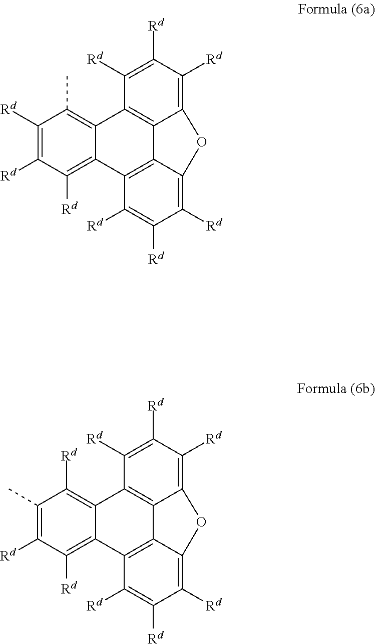
Y is the same or different at each instance and is selected from O, S and C(R^{k})₂;
Ar¹, Ar² are the same or different at each instance and are each independently an aromatic ring system having 6 to 40 aromatic ring atoms or a heteroaromatic ring system having 5 to 40 aromatic ring atoms, each of which may be substituted by one or more R¹ radicals;
R, R^{a}, R^{b} are the same or different at each instance and are each independently H, D, F, Cl, Br, I, N(Ar′)₂, N(R¹)₂, OAr′, SAr′, B(OR¹)₂, CHO, C(═O)R¹, CR¹═C(R¹)₂, CN, C(═O)OR¹, C(═O)NR¹, Si(R¹)₃, NO₂, P(═O)(R¹)₂, OSO₂R¹, OR¹, S(═O)R¹, S(═O)₂R¹, SR¹, a straight-chain alkyl group having 1 to 20 carbon atoms or an alkenyl or alkynyl group having 2 to 20 carbon atoms or a branched or cyclic alkyl group having 3 to 20 carbon atoms, where the alkyl, alkenyl or alkynyl group may in each case be substituted by one or more R¹ radicals, where one or more nonadjacent CH₂ groups may be replaced by —R¹C═CR¹—, —C≡C—, Si(R¹)₂, CONR¹, C—O, C═S, —C(═O)O—, P(═O)(R¹), O, S, SO or SO₂, or an aromatic, or heteroaromatic ring system having 5 to 40 aromatic ring atoms, each of which may be substituted by one or more R¹ radicals, where two or more R and/or R^{a} and/or R^{b} radicals bonded to the same cycle may together form an aliphatic, heteroaliphatic or aromatic ring system that may be substituted by one or more R¹ radicals, and where two R and/or R^{a} and/or R^{b} radicals bonded to the same carbon, silicon, germanium or tin atom may together form a monocyclic or polycyclic, aliphatic or aromatic ring system that may be substituted by one or more R¹ radicals;
R^{c} is the same or different at each instance and is H, D, F, Cl, Br, I, N(Ar′)₂, N(R¹)₂, OAr′, SAr′, B(OR¹)₂, CHO, C(═O)R¹, CR¹═C(R¹)₂, CN, C(═O)OR¹, C(═O)NR¹, Si(R¹)₃, NO₂, P(═O)(R¹)₂, OSO₂R¹, OR¹, S(═O)R¹, S(═O)₂R¹, SR¹, a straight-chain alkyl group having 1 to 20 carbon atoms or an alkenyl or alkynyl group having 2 to 20 carbon atoms or a branched or cyclic alkyl group having 3 to 20 carbon atoms, where the alkyl, alkenyl or alkynyl group may in each case be substituted by one or more R¹ radicals, where one or more nonadjacent CH₂ groups may be replaced by —R¹C═CR¹—, —C≡C—, Si(R¹)₂, NR¹, CONR¹, C═O, C═S, —C(═O)O—, P(═O)(R¹), O, S, SO or SO₂, or an aromatic or heteroaromatic ring system having 5 to 40 aromatic ring atoms, each of which may be substituted by one or more R¹ radicals, where two or more R^{c}, R^{e}, R^{f}, R^{g}, R^{h} or Rⁱ radicals bonded to the same cycle may together form an aliphatic, heteroaliphatic, aromatic or heteroaromatic ring system that may be substituted by one or more R¹ radicals, and where two R^{c}, R^{e}, R^{f}, R^{g}, R^{h} or Rⁱ radicals bonded to the same carbon, silicon, germanium or tin atom may together form a monocyclic or polycyclic, aliphatic, aromatic or heteroaromatic ring system that may be substituted by one or more R¹ radicals,
R^{d} is the same or different at each instance and is H, D, F, Cl, Br, I, N(Ar′)₂, N(R³)₂, OAr′, SAr′, B(OR³)₂, CHO, C(═O)R³, CR³═C(R³)₂, CN, C(═O)OR³, C(═O)NR³, Si(R³)₃, NO₂, P(═O)(R³)₂, OSO₂R³, S(═O)R³, S(═O)₂R³, SR³, a straight-chain alkyl group having 1 to 20 carbon atoms or an alkenyl or alkynyl group having 2 to 20 carbon atoms or a branched or cyclic alkyl group having 3 to 20 carbon atoms, where the alkyl, alkenyl or alkynyl group may be substituted in each case by one or more R¹ radicals, where one or more nonadjacent CH₂ groups may be replaced by —R³C═CR³—, —C≡C—, Si(R³)₂, NR³, CONR³, C═O, C═S, —C(═O)O—, P(═O)(R³), O, S, SO or SO₂, or an aromatic or heteroaromatic ring system which has 5 to 40 aromatic ring atoms and may be substituted in each case by one or more R³ radicals, where two or more R^{d} radicals bonded to the same cycle may together form an aliphatic, heteroaliphatic, aromatic or heteroaromatic ring system which may be substituted by one or more R¹ radicals, and where two R^{d} radicals bonded to the same carbon, silicon, germanium or tin atom may together form a monocyclic or polycyclic, aliphatic, aromatic or heteroaromatic ring system which may be substituted by one or more R³ radicals;
Ar′ is the same or different at each instance and is an aromatic ring system having 6 to 40 aromatic ring atoms or a heteroaromatic ring system having 5 to 40 aromatic ring atoms, which may be substituted by one or more R¹ radicals;
R^{e}, R^{f}, R^{g}, R^{h} are the same or different at each instance and are each independently H, D, F, CI, Br, I, N(Ar′)₂, N(R¹)₂, OAr′, SAr′, B(OR¹)₂, CHO, C(═O)R¹, CR¹═C(R¹)₂, CN, C(═O)OR¹, C(═O)NR¹, Si(R¹)₃, NO₂, P(═O)(R¹)₂, OSO₂R¹, OR¹, S(═O)R¹, S(═O) R1 SR¹, a straight-chain alkyl group having 1 to 20 carbon atoms or an alkenyl or alkynyl group having 2 to 20 carbon atoms or a branched or cyclic alkyl group having 3 to 20 carbon atoms, where the alkyl, alkenyl or alkynyl group may be substituted in each case by one or more R′ radicals, where one or more nonadjacent CH₂ groups may be replaced by —R¹C═CR¹—, —C≡C—, Si(R¹)₂, NR¹, CONR¹, C═O, C═S, —C(═O)O—, P(═O)(R¹), O, S, SO or SO₂, or an aromatic or heteroaromatic ring system which has 5 to 40 aromatic ring atoms and may be substituted in each case by one or more R¹ radicals;
Rⁱ, R^{j} are the same or different at each instance and are each independently H, D, F, Cl, Br, I, N(Ar′)₂, N(R¹)₂, OAr′, SAr′, B(OR¹)₂, CHO, C(═O)R¹, CR¹═C(R¹)₂, CN, C(═O)OR¹, C(═O)NR¹, Si(R¹)₃, NO₂, P(═O)(R¹)₂, OSO₂R¹, OR¹, S(═O)R¹, S(═O)₂R¹, SR¹, a straight-chain alkyl group having 1 to 20 carbon atoms or an alkenyl or alkynyl group having 2 to 20 carbon atoms or a branched or cyclic alkyl group having 3 to 20 carbon atoms, where the alkyl, alkenyl or alkynyl group may in each case be substituted by one or more R¹ radicals, where one or more nonadjacent CH₂ groups may be replaced by —R¹C═CR¹, —C≡C—, Si(R¹)₂, NR¹, CONR¹, C═O, C═S, —C(═O)O—, P(═O)(R¹), O, S, SO or SO₂, or an aromatic or heteroaromatic ring system having 5 to 40 aromatic ring atoms, each of which may be substituted by one or more R¹ radicals, where two or more Rⁱ or R^{j} radicals bonded to the same cycle may together form an aliphatic, heteroaliphatic, aromatic or heteroaromatic ring system that may be substituted by one or more R¹ radicals, and where two Rⁱ or R^{j} radicals bonded to the same carbon, silicon, germanium or tin atom may together form a monocyclic or polycyclic, aliphatic, aromatic or heteroaromatic ring system that may be substituted by one or more R¹ radicals;
R^{k}, R^{l} are the same or different at each instance and are D, F, I, B(OR²)₂, N(R²)₂, CHO, C(═O)R², CR²═C(R²)₂, CN, C(═O)OR², Si(R²)₃, NO₂, P(═O)(R²)₂, OSO₂R², SR², OR², S(═O)R², S(═O)₂R², a straight-chain alkyl group having 1 to 20 carbon atoms or an alkenyl or alkynyl group having 2 to 20 carbon atoms or a branched or cyclic alkyl group having 3 to 20 carbon atoms, where the alkyl, alkenyl or alkynyl group may in each case be substituted by one or more R² radicals and where one or more CH₂ groups in the abovementioned groups may be replaced by —R²C═CR²—, —C≡C—, Si(R²)₂, C═O, C═S, —C(═O)O—, NR², CONR², P(═O)(R²), O, S, SO or SO₂, and where one or more hydrogen atoms in the abovementioned groups may be replaced by D, F, Cl, Br, I, CN or NO₂, or an aromatic or heteroaromatic ring system which has 5 to 30 aromatic ring atoms and may be substituted in each case by one or more R² radicals, where two or more R^{k} or R^{l} radicals together may form an aliphatic, heteroaliphatic, aromatic or heteroaromatic ring system;
R² is the same or different at each instance and is D, F, CN or an aliphatic, aromatic or heteroaromatic organic radical having 1 to 20 carbon atoms, in which one or more hydrogen atoms may also be replaced by D or F; at the same time, two or more R² substituents may be joined to one another and may form a ring;
R³ is the same or different at each instance and is D, CN or an aliphatic, aromatic or heteroaromatic organic radical having 1 to 20 carbon atoms; at the same time, two or more R³ substituents may be joined to one another and may form a ring;
l, m, p, q are the same or different at each instance and are independently 0, 1, 2 or 3;
n, o, r, z are the same or different at each instance and are independently 0, 1, 2, 3 or 4, and
y at each instance is independently 0 or 1.

14. The composition as claimed in claim 13, wherein the compounds of the formula (1) are selected from compounds of the formulae (1-1a) to (1-1t): 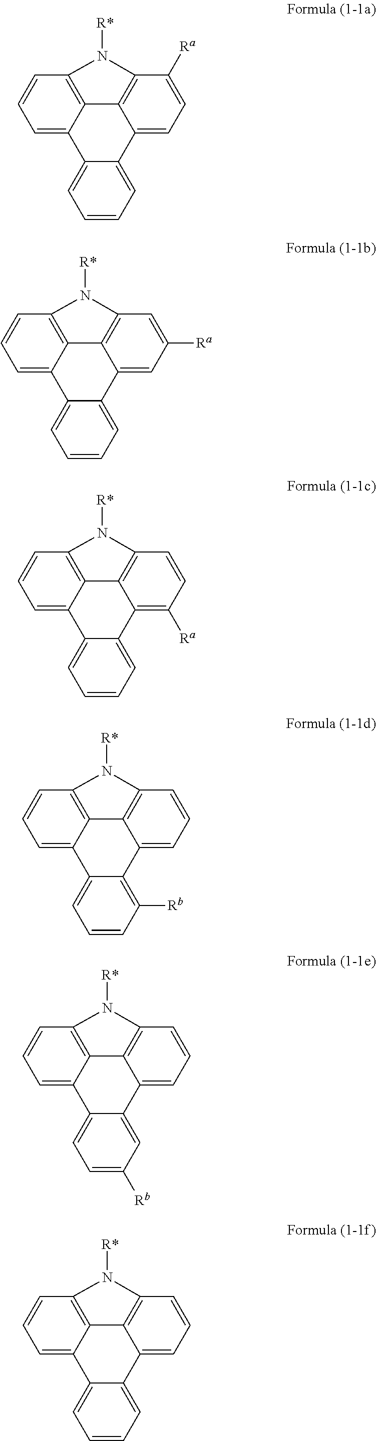 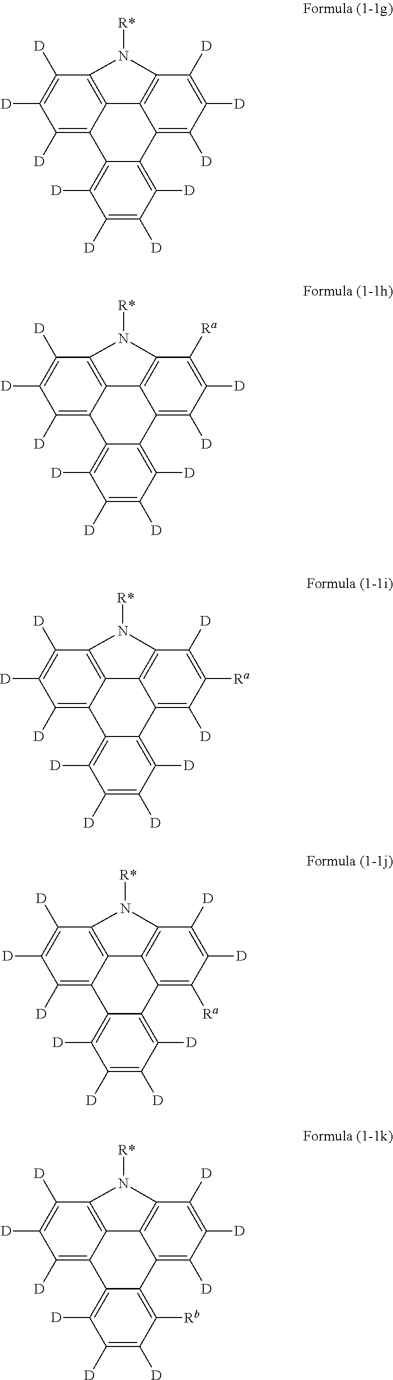 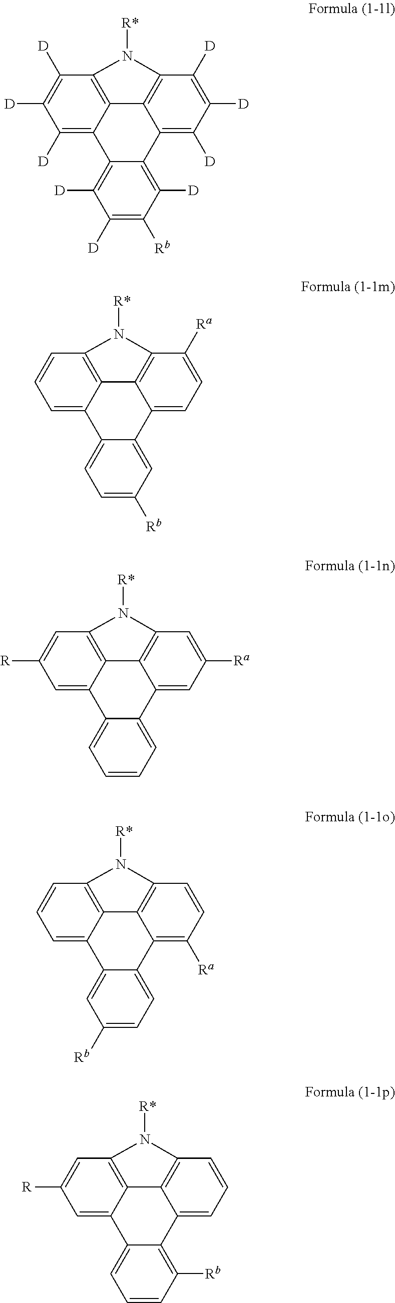 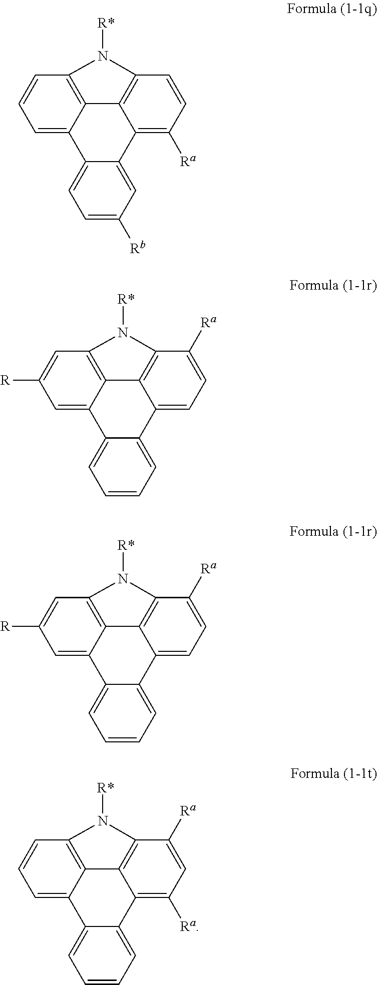
where R* is a group of the following formulae (1b), (1c) or (1d): 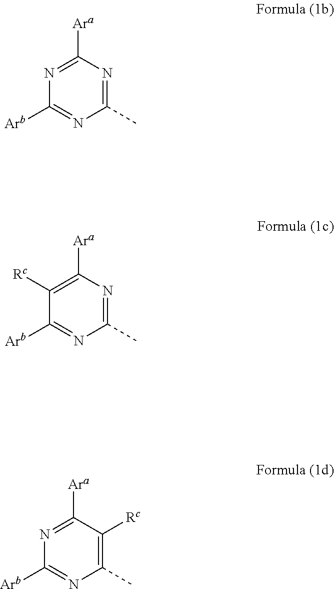
and the dashed bond represents the linkage of the group of the formula (1b), (1c) or (1d) to the nitrogen atom in the main structure of the formulae (1-1a) to (1-1t), and where the symbols R, R^{a}, R^{b}, R^{c}, Ar^{a} and Ar^{b} used have the definition given in claim 13.

15. The composition as claimed in claim 13, wherein the compounds of the formula (2) or of the formula (3) are selected from compounds of the formulae (2-1), (2-2), (2-3), (2-4), (3-1), (3-2), (3-3) and (3-4): 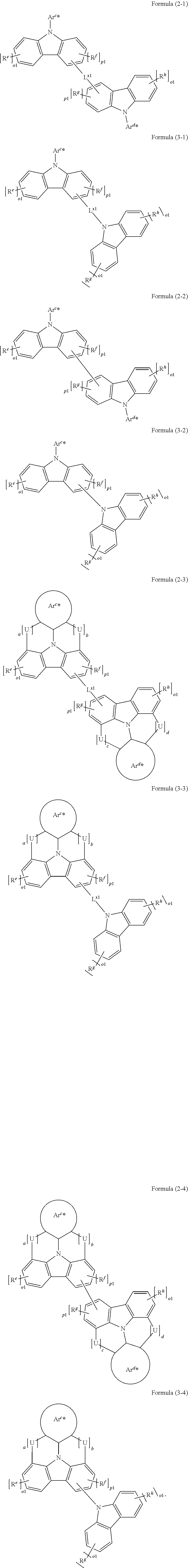
wherein Ar^{c*}, Ar^{d*}, Ar^{c}, Ar^{d}, R^{d}, R^{e}, R^{f}, R^{g}, R^{h}, Ar′ and R¹ have the definition given in claim 13, and where:
L^{x1} is the same or different at each instance and is an aromatic ring system having 6 to 40 aromatic ring atoms or a heteroaromatic ring system having 5 to 40 aromatic ring atoms, which may be substituted in each case by one or more R¹ radicals;
U is the same or different at each instance and is independently a single bond, O, S, NAr′ or C(R¹)₂;
a, b, c, d are the same or different at each instance and are independently 0 or 1, with the proviso that the sum of a+b=1 and the sum of c+d=0 or 1, or that the sum of c+d=1 and the sum of a+b=0;
o1 is the same or different at each instance and is independently 0, 1, 2, 3 or 4; and
p1 is the same or different at each instance and is independently 0, 1, 2 or 3.

16. The composition as claimed in claim 13, wherein the compounds of the formulae (2), (3), (4), (2-1), (2-2), (2-3), (2-4), (3-1), (3-2), (3-3) and (3-4) are selected from compounds of the formulae (2-1-1) to (2-1-3), (3-1-1) to (3-1-3), (2-2-1) to (2-2-16), (3-2-1) to (3-2- 4) and (4-1) to (4-6), (2-3-1) to (2-3-5), (3-3-1) to (3-3-3), (2-4-1) to (2-4-5) and (3-4-1) to (3-4-2): 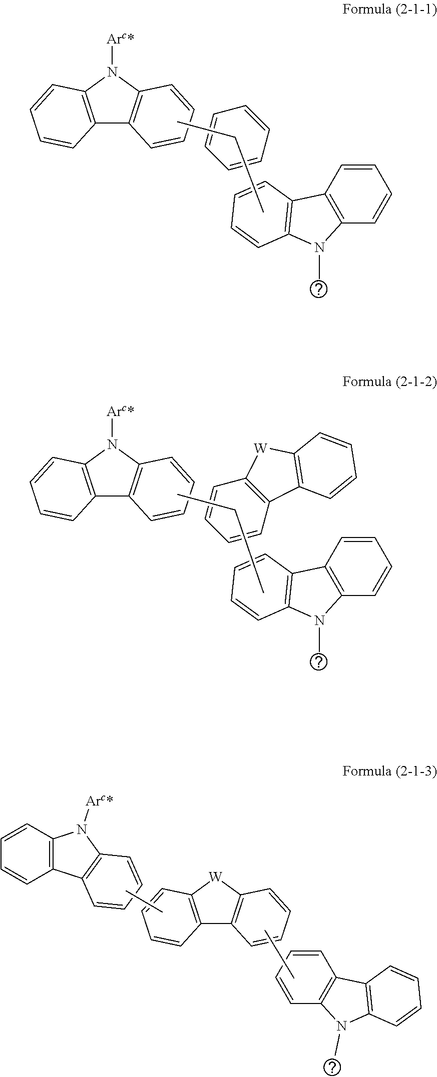 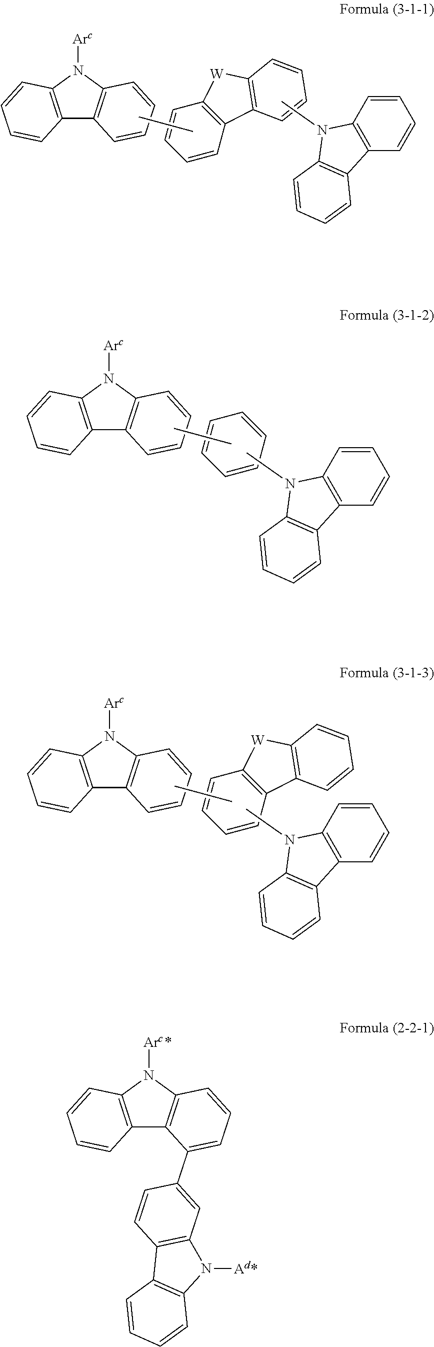 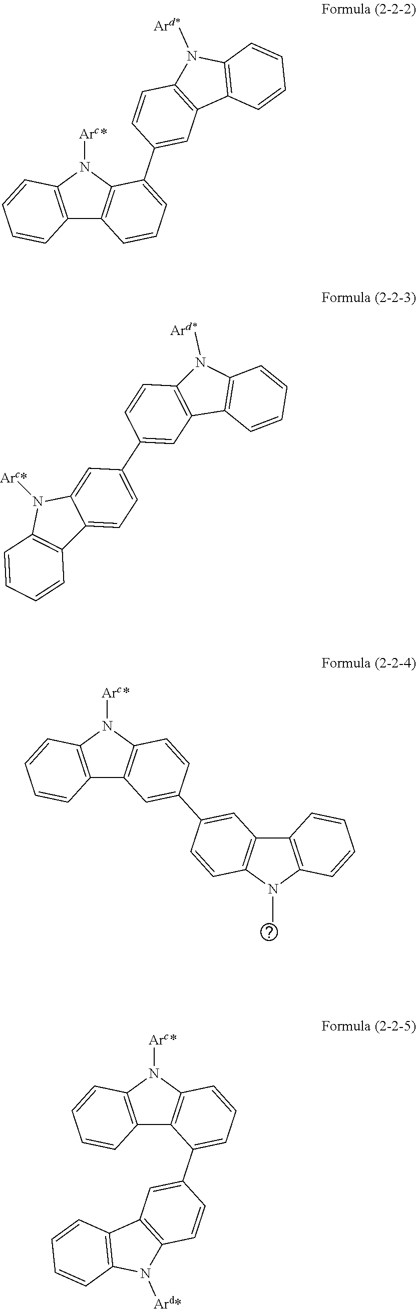 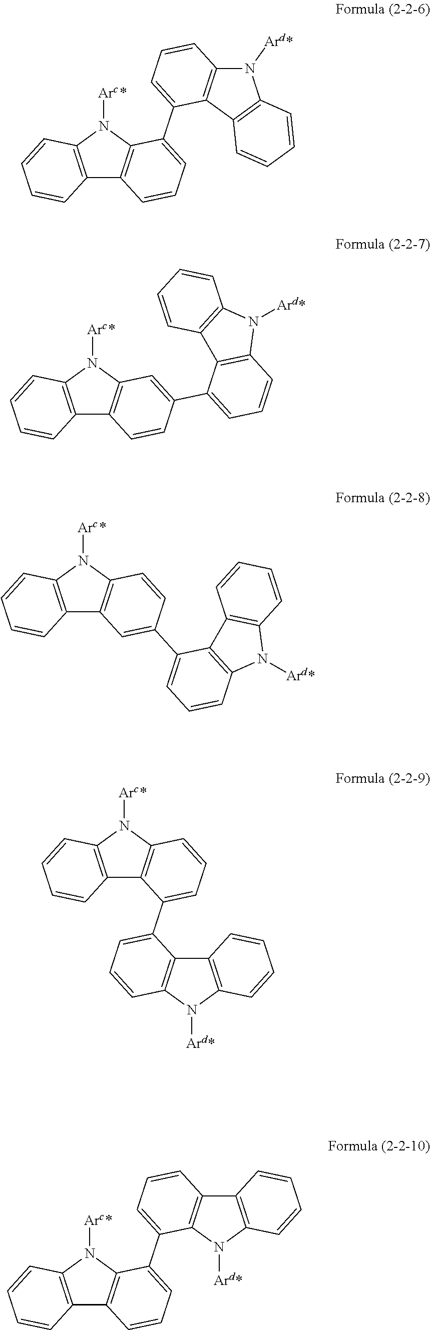 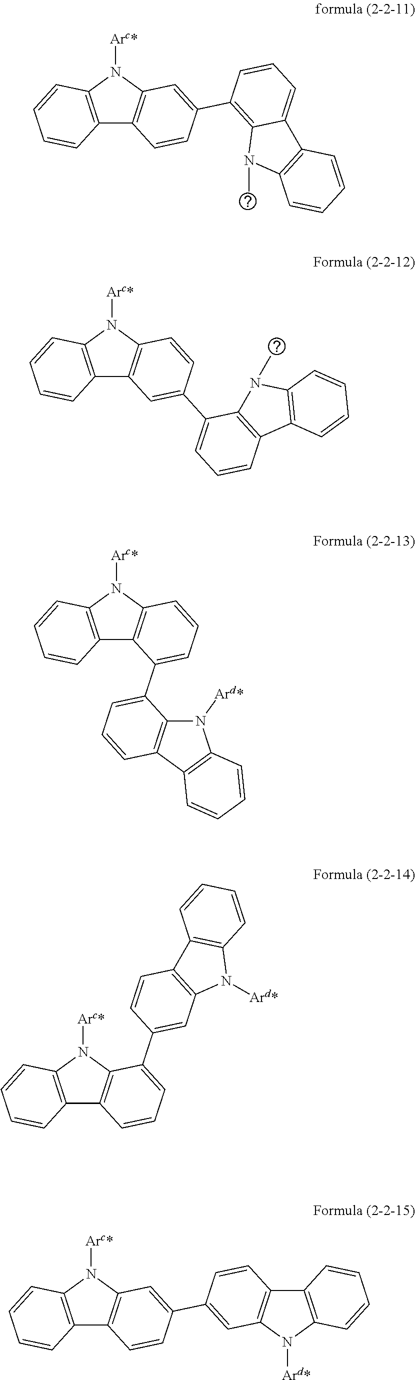 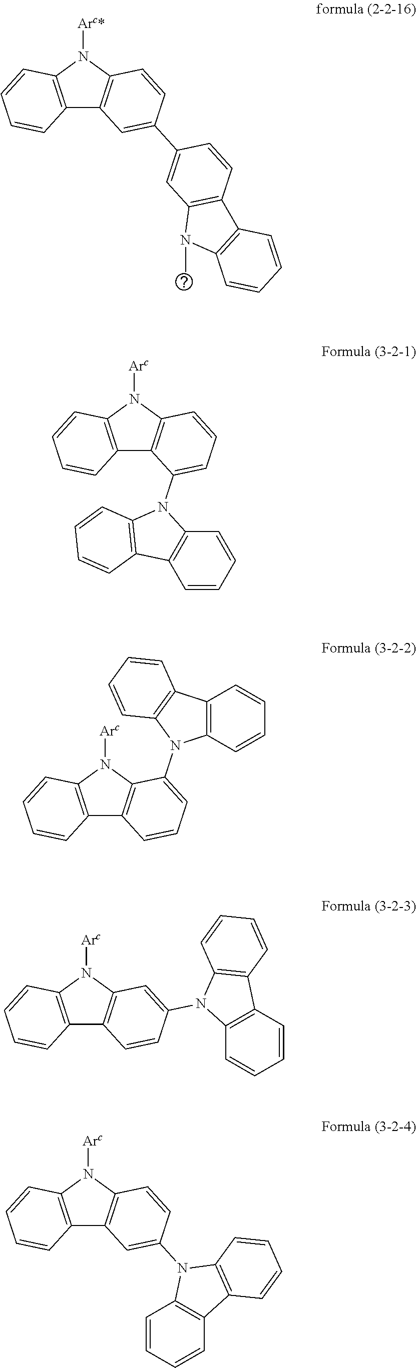 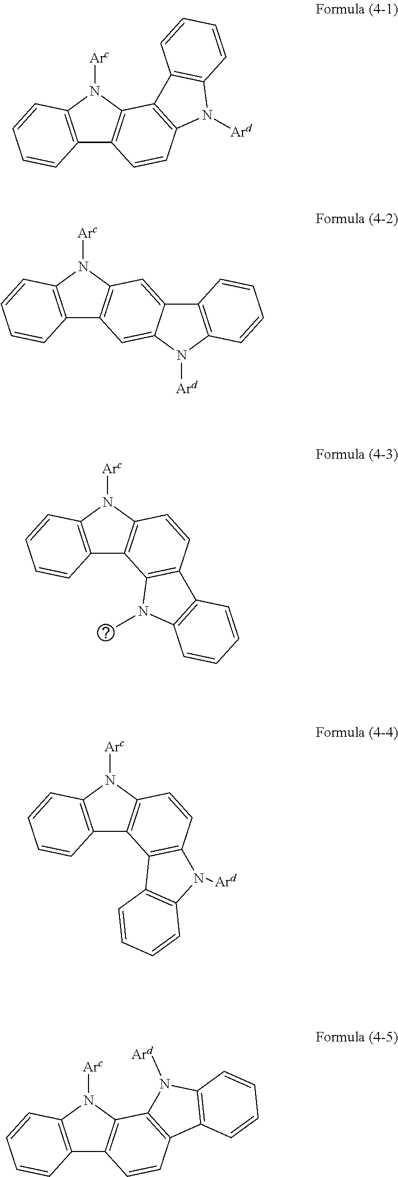 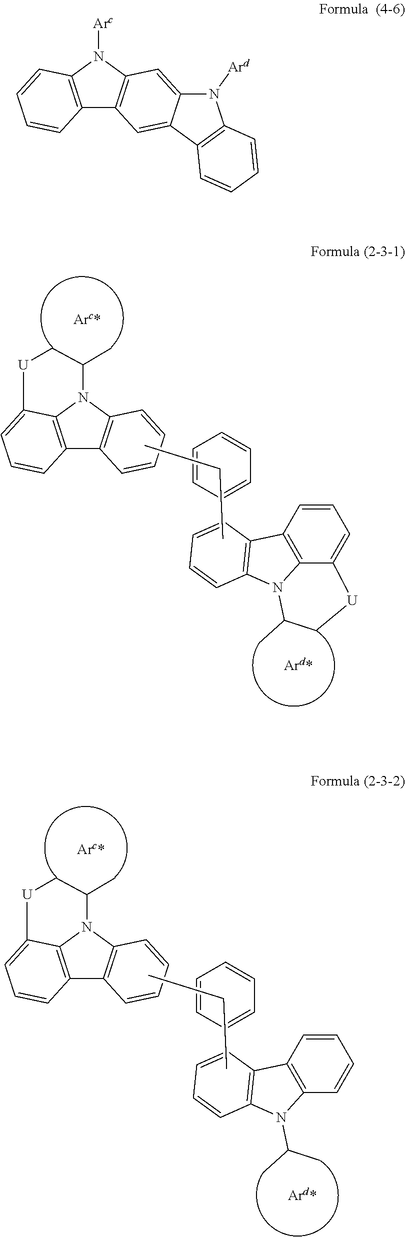 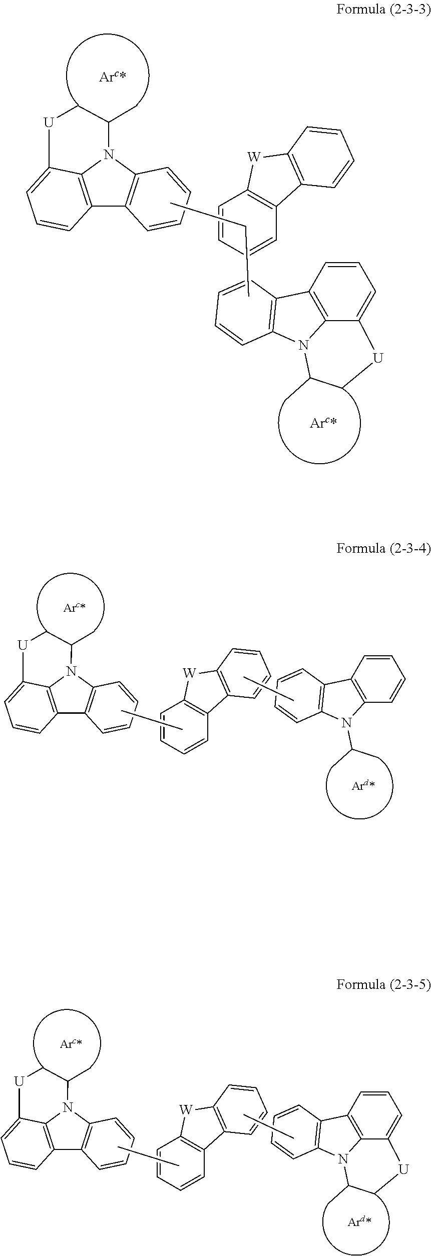 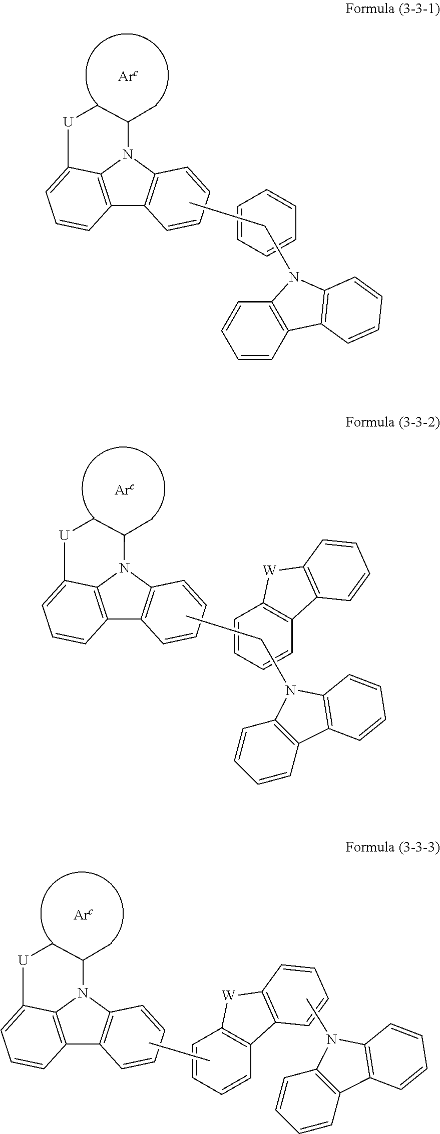 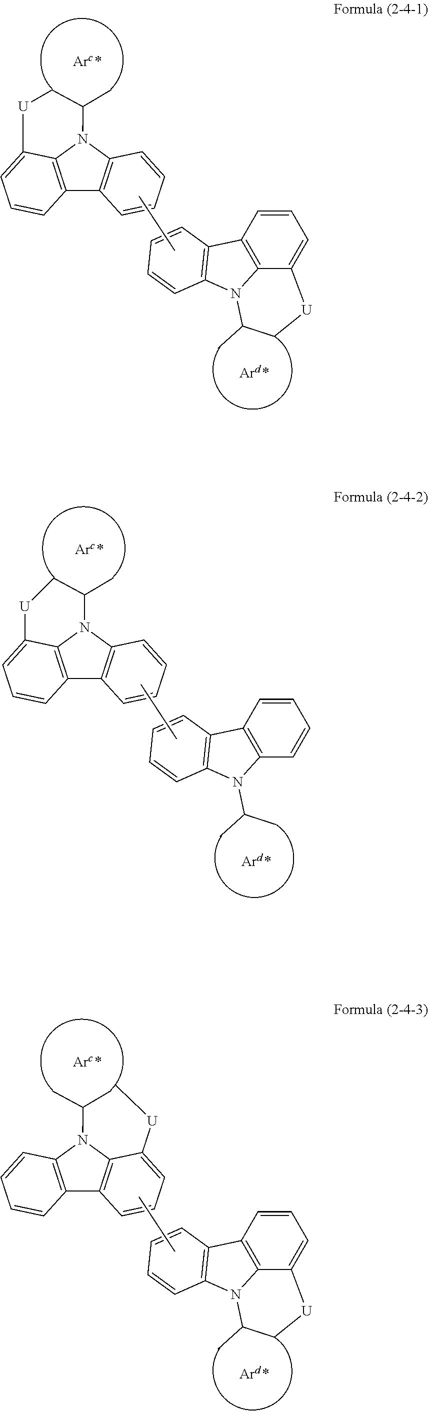 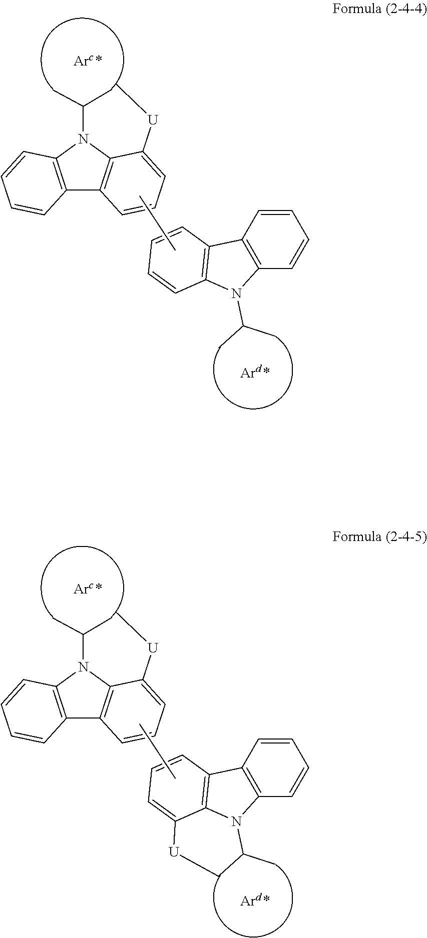 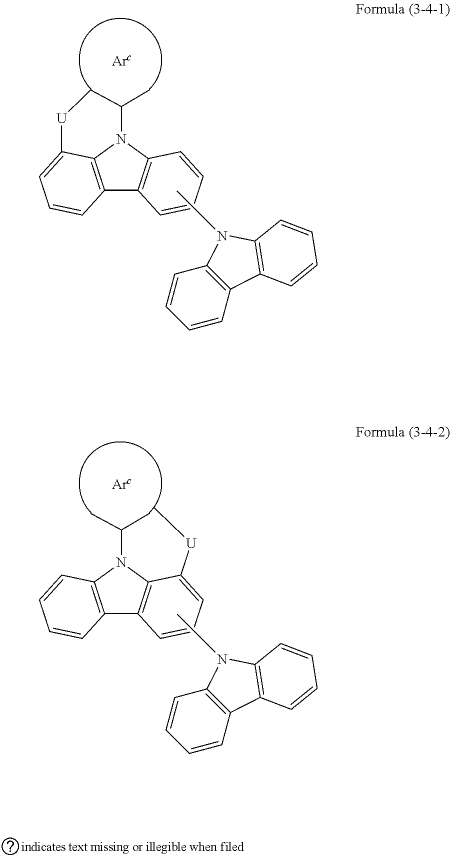
where the symbols Ar^{c*}, Ar^{d*}, Ar^{c}, Ar^{d}, Ar′ and U have the definitions given in claim 13, W is O, S or NAr′, and the hydrogen atoms in the base skeleton of the compounds may be fully or partly replaced by deuterium.

17. A method comprising incorporating the composition as claimed in claim 13 in an organic electronic device.

18. An organic electronic device comprising at least one composition as claimed in claim 13 in at least one organic layer.

19. The device as claimed in claim 18, wherein the device is selected from the group of organic integrated circuits (OICs), organic field-effect transistors (OFETs), organic thin-film transistors (OTFTs), organic solar cells (OSCs), organic optical detectors, organic photoreceptors, organic light-emitting transistors (OLETs), organic field-quench devices (OFQDs), organic light-emitting electrochemical cells (OLECs, LECs, LEECs), organic laser diodes (O-lasers), and organic light-emitting diodes (OLEDs).

20. The device as claimed in claim 18, wherein the device comprises the composition as in an emission layer (EML), in an electron transport layer (ETL), in an electron injection layer (EIL) and/or in a hole blocker layer (HBL).

21. The device as claimed in claim 18, wherein the device comprises the composition as in the emission layer together with a phosphorescent emitter.

22. A process for producing a device as claimed in claim 18, wherein at least one organic layer comprising the composition is applied by gas phase deposition or from solution.

23. The process as claimed in claim 22, wherein the composition comprising at least one compound of the formula (1) and the at least one compound of the formula (2) or of the formula (3) or of the formula (4) or of the formula (5) are deposited from the gas phase successively or simultaneously from at least two material sources, optionally together with further materials, and form the organic layer.

24. The process as claimed in claim 22, wherein the composition comprising at least one compound of the formula (1) and the at least one compound of the formula (2) or of the formula (3) or of the formula (4) or of the formula (5) are deposited from the gas phase successively or simultaneously from a material source, together with at least one phosphorescent emitter, and form the light-emitting layer.
